(19) United States
(12) Patent Application Publication
(10) Pub. No.: US 2010/0210633 A1
(43) Pub. Date: Aug. 19, 2010

(54) CARBOXAMIDE COMPOUNDS AND THEIR USE

(75) Inventors: Jian Lin, Acton, MA (US); Dongli Chen, Brighton, MA (US); Steffi Koerner, Medford, MA (US); Rosa E. Melendez, Woburn, MA (US); Pradyumma Mohanty, Woburn, MA (US); Efrat Ben-Zeev, K. Motzkin (IL); Merav Fichman, Modi'i (IL); Yael Marantz, Kadima (IL); Oren Becker, Mevaseret Zion (IL); Dilara McCauley, Cambridge, MA (US); Pini Orbach, Tel Mond (IL); Ashis K. Saha, Stow, MA (US); Sharon Shacham, Chestnut Hill, MA (US); Michael Xie, Natick, MA (US)

Correspondence Address: GOODWIN PROCTER LLP;PATENT ADMINISTRATOR, 53 STATE STREET, EXCHANGE PLACE, BOSTON, MA, 02109-2881, US

(73) Assignee: EPIX Delaware, Inc., Lexington, MA (US)

(21) Appl. No.: 12/445,134

(22) PCT Filed: Oct. 12, 2007
(86) PCT No.: PCT/US07/21917

§ 371 Date: Mar. 25, 2010

Related U.S. Application Data
(60) Provisional application No. 60/851,338, filed on Oct. 12, 2006.

(51) Int. Cl. A61K 31/541; C07D 405/06; A61K 31/453; A61P 3/10; A61P 3/04; A61P 37/06; A61P 17/06; A61P 9/10; A61P 13/12; A61P 1/16; C07D 211/62; A61K 31/445; C07D 207/16; A61K 31/40; A61K 31/4525; C07D 413/10; A61K 31/454; C07D 405/14; A61K 31/496; C07D 401/06; A61K 31/4545; C07D 401/04; C07D 417/06; C07D 413/06; A61K 31/5377

(52) U.S. Cl. 514/227.8; 546/207; 514/326; 546/225; 514/330; 548/537; 514/423; 546/197; 514/321; 546/199; 514/322; 544/364; 514/253.11; 546/189; 514/316; 546/194; 514/318; 544/58.2; 544/130; 514/235.5; 514/236.8

(57) ABSTRACT

Chemokine receptor antagonists, in particular, compounds of Formula (I) that act as antagonists of the chemokine CCR2 receptor, including pharmaceutical compositions and uses thereof to treat or prevent diseases associated with monocyte accumulation, lymphocyte accumulation or leukocyte accumulation are described herein.

### RELATED APPLICATIONS

This application claims priority to U.S. Ser. No. 60/851,338, filed on Oct. 12, 2006, which is hereby incorporated by reference in its entirety.

### FIELD OF THE INVENTION

The invention generally relates to the field of chemokine receptor antagonists, in particular, compounds that act as antagonists of the chemokine CCR2 receptor, including pharmaceutical compositions; and uses thereof to treat or prevent diseases associated with, e.g., monocyte accumulation, lymphocyte accumulation or leukocyte accumulation.

### BACKGROUND OF THE INVENTION

Leukocyte migration and transport from blood vessels into diseased tissues appears to be a critical component to the initiation of normal disease-fighting inflammatory responses. This process—leukocyte recruitment—is also related to the onset and progression of life-threatening inflammatory and debilitating autoimmune diseases.

The resulting pathology of these diseases derives from the attack of the body's immune system defenses on normal tissues. Accordingly, preventing and blocking leukocytes recruitment to target tissues in inflammatory and autoimmune disease would be a highly effective approach to therapeutic intervention.

The different classes of leukocyte cells involved in cellular immune responses include monocytes, lymphocytes, neutrophils, eosinophils and basophils. In most cases, lymphocytes are the leukocyte class that initiates, coordinates, and maintains chronic inflammatory responses, and thus are generally the most important class of cells to block from entering inflammatory sites. Lymphocytes attract monocytes to the tissue sites, which—with lymphocytes—are responsible for most of the actual tissue damage that occurs in inflammatory disease. Lymphocyte and/or monocyte infiltration is known to lead to a wide range of chronic, autoimmune diseases, and also organ transplant rejection. These diseases include rheumatoid arthritis, chronic contact dermatitis, inflammatory bowel disease, lupus, systemic lupus erythematosus, multiple sclerosis, atherosclerosis, psoriasis, sarcoidosis, idiopathic pulmonary fibrosis, dermatomyositis, skin pemphigoid and related diseases, (e.g., pemphigus vulgaris, p. foliacious, p. erythematosis), glomerulonephritides, vasculitides, hepatitis, diabetes, allograft rejection, and graft-versus host disease.

The process, by which leukocytes leave the bloodstream and accumulate at inflammatory sites and start a disease, has at least three steps which have been described as (1) rolling, (2) activation/firm adhesion and (3) transendothelial migration. The second step is mediated at the molecular level by chemoattractant receptors. Chemoattractant receptors on the surface of leukocytes then bind chemoattractant cytokines which are secreted by cells at the site of damage or infection.

Receptor binding activates leukocytes, increases the adhesiveness of the adhesion molecules that mediate transendothelial migration, and promotes directed migration of the cells toward the source of the chemoattractant cytokine.

Chemotactic cytokines (leukocyte chemoattractant/activating factors, also known as chemokines, intercrines and SIS cytokines), are a group of 6-15 kDa inflammatory/immunomodulatory polypeptide factors that are released by a wide variety of cells such as macrophages, monocytes, eosinophils, neutrophiles, fibroblasts, vascular endothelial cells, smooth muscle cells, and mast cells, at inflammatory sites.

Chemokines have the ability to stimulate directed cell migration, a process known as chemotaxis. Each chemokine contains four cysteine residues (C) and two internal disulfide bonds. Chemokines can be grouped into two subfamilies, based on whether the two amino terminal cysteine residues are immediately adjacent (“CC”) or separated by one amino acid (“CXC”). These differences correlate with the organization of the two subfamilies into separate gene clusters. Within each gene cluster, the chemokines typically show sequence similarities between 25 to 60%. The CXC chemokines such as interleukin-8 (IL-8), neutrophil-activating protein-2 (NAP-2) and melanoma growth stimulatory activity protein (MGSA) are chemotactic primarily for neutrophils and T lymphocytes. The CC chemokines, such as RANTES, MIP-1a, MIP-1p, the monocyte chemotactic proteins (MCP-1, MCP-2, MCP-3, MCP-4, and MCP-5) and the eotaxins (−1 and −2) are chemotactic for, among other cell types, macrophages, T lymphocytes, eosinophils, dendritic cells, and basophils. Chemokines that do not fall into either of the major chemokine subfamilies include lymphotactin-1, lymphotactin-2 (both C chemokines), and fractalkine (a CXXXC chemokine.)

MCP-1 (also known as MCAF (Macrophage Chemotactic and Activating Factor), or JE) is a CC chemokine produced by monocytes/macrophages, smooth muscle cells, fbroblasts, and vascular endothelial cells. It causes cell migration and cell adhesion of monocytes, memory T lymphocytes, T lymphocytes and natural killer cells, as well as mediating histamine release by basophils. High expression of MCP-1 has been reported in diseases where accumulation of monocyte/macrophage and/or T cells is thought to be important in the initiation or progression of diseases, such as atherosclerosis, rheumatoid arthritis, nephritis, nephropathy, pulmonary fibrosis, pulmonary sarcoidosis, asthma, multiple sclerosis, psoriasis, inflammatory bowel disease, myocarditis, endometriosis, intraperitoneal adhesion, congestive heart failure, chronic liver disease, viral meningitis, Kawasaki disease and sepsis.

Furthermore, anti-MCP-1 antibody has been reported to show an inhibitory effect or a therapeutic effect in animal models of rheumatoid arthritis, multiple sclerosis, nephritis, asthma, atherosclerosis, delayed type hypersensitivity, pulmonary hypertension, and intraperitoneal adhesion. A peptide antagonist of MCP-1, MCP-1 (9-76), has been also reported to inhibit arthritis in the mouse model, as well as studies in MCP-1-deficient mice have shown that MCP-1 is essential for monocyte recruitment in vivo.

The published literature indicates that chemokines such as MCP-1 and MIP-1a attract monocytes and lymphocytes to disease sites and mediate their activation and thus are thought to be intimately involved in the initiation, progression and maintenance of diseases deeply involving monocytes and lymphocytes, such as atherosclerosis, restenosis, rheumatoid arthritis, psoriasis, asthma, ulcerative colitis, nephritis (nephropathy), multiple sclerosis, pulmonary fibrosis, myocarditis, hepatitis, pancreatitis, sarcoidosis, Crohn's disease, endometriosis, congestive heart failure, viral meningitis, cerebral infarction, neuropathy, Kawasaki disease, and sepsis. The chemokines bind to specific cell-surface receptors belonging to the family of G protein-coupled seven-transmembrane-domain proteins which are termed “chemokine receptors.” On binding their cognate ligands, chemokine receptors transduce an intracellular signal through the associated trimeric G proteins, resulting in, among other responses, a rapid increase in intracellular calcium concentration, changes in cell shape, increased expression of cellular adhesion molecules, degranulation, and promotion of cell migration.

Genes encoding receptors of specific chemokines have been cloned, and it is now known that these receptors are G protein-coupled seven-transmembrane receptors present on various leukocyte populations. So far, at least five CXC chemokine receptors (CXCR1 CXCR5) and eight CC chemokine receptors (CCR1-CCR8) have been identified. For example, IL-8 is a ligand for CXCR1 and CXCR2; MIP-1a is a ligand for CCR1 and CCR5, and MCP-I is a ligand for CCR2A and CCR2B. It has been reported that lung inflammation and granuroma formation are suppressed in CCR1-deficient mice, and that recruitment of macrophages and formation of atherosclerotic lesion decreased in CCR2-deficient mice. See, e.g., Murdoch et al., “Chemokine receptors and their role in inflammation and infectious diseases,” Blood 95(10):3032-3043 (2000), which is incorporated by reference herein.

CCR2 (also termed CKR-2, MCP-1RA or MC1RB) is predominantly expressed on monocytes and macrophages, and is necessary for macrophage-dependent inflammation (Bruhl et al. 1970). CCR2 is a G protein-coupled receptor (GPCR) which binds with high affinity (Kd of 1 nM) to several members of the MCP family of chemokines (CCL2, CCL7, CCL8, etc.), eliciting a chemotactic signal that results in directed migration of the receptor-bearing cells (Dunzendorfer et al. 2001).

CCR2 is implicated in the pathogenesis of several inflammatory diseases such as rheumatoid arthritis, multiple sclerosis and atherosclerosis (Rodriguez-Frade et al. 2005). The critical role of the CCL2-CCR2 pathway as a modulator of the tissue influx of monocytes was demonstrated in mice deficient in the receptor, CCR2, or the ligand, CCL2, which are phenotypically normal, but show a selective defect in the migration of macrophages to sites of inflammation (Boring et al. 1997; Lu et al. 1998).

It was also recently shown that mRNA levels of CCR2 increase with peak inflammation in rat adjuvant-induced arthritis (AIA), a model for rheumatoid arthritis (Shahrara et al. 2003). Moreover, a small molecule CCR2 antagonist with high affinity for the mouse CCR2 receptor was shown to reduce disease in mice subjected to experimental autoimmune encephalomyelitis, a model of multiple sclerosis, as well as a rat model of inflammatory arthritis (Brodmerkel et al. 2005) See also deBoer, “Perspectives for Cytokine Antagonist therapy in COPD”, Drug Discov. Today, 10(2):93-106 (2005), which is incorporated by reference herein. Taken together, these results support the ability to treat of chronic inflammatory diseases with chemical antagonists of CCR2.

### SUMMARY OF THE INVENTION

Drugs which inhibit the binding of chemokines to their receptors, e.g., chemokine receptor antagonists, are believed to be useful as pharmaceutical agents which inhibit the action of chemokines on their target cells. The identification of compounds that modulate the function of CCR2 represents an excellent drug design approach to the development of pharmacological agents for the treatment of inflammatory conditions and diseases associated with CCR2 activation, such as rheumatoid arthritis, lupus and other inflammatory diseases.

The invention is directed to chemokine receptor modulators, e.g., antagonists, and their use as medicinal agents. The invention further relates to novel compounds and medical methods of treatment of inflammation, and other disorders especially those associated with lymphocyte or monocyte accumulation such as atherosclerosis, rheumatoid arthritis, lupus, graft versus host diseases and/or transplant rejection. The invention also relates to novel compounds and medical methods of treatment of metabolic syndrome, Non-Insulin Dependant Type II Diabetes (NIDDM), and obesity, as well as other diseases or conditions disclosed herein.

More particularly, the invention relates to compounds of Formula I:

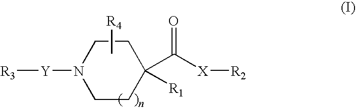

or enantiomers, diastereomers, enantiomerically enriched mixtures, racemic mixtures thereof, prodrugs, crystalline forms, non-crystalline forms, amorphous forms, solvates, metabolites, and pharmaceutically acceptable salts thereof, wherein:

R¹ may be hydrogen; unsubstituted alkyl, alkoxyalkyl, alkoxyphenyl, alkylthioalkyl, or alkylamino; a C₃₋₆ alicyclic or heterocyclic ring; or a heteroaryl ring which, when substituted, has no more than three R⁵ substituents;

X may be NH, NR⁹, NHCH₂, NR⁹CH₂, CHR⁹, CH₂, NHCO, NHSO₂, or a direct bond, where R⁹ is C₁₋₂alkyl or C₁₋₂alkenyl;

R² may be an alicyclic, aromatic (e.g., benzyl), heterocyclic or heteroaromatic ring; which ring, when substituted, has no more than three R⁵ substituents;

Y may be an unsubstituted C₁₋₃ alkyl, CO, SO₂, NHCO, NHSO₂, NH═C, R⁹NH═C, an alicyclic, aromatic, heterocyclic or heteroaromatic ring; which ring, when substituted, has no more than three R⁵ substituents; or an unsubstituted cyclic or bicyclic ring optionally having partial aromaticity; or a direct bond;

R³ may be hydrogen; an alkyl, alkenyl, or alkynyl group; or an alicyclic, aromatic, heterocyclic and heteroaromatic ring; which ring, when substituted, has no more than three R⁵ substituents, and provided that Y and R₃ are not both a ring;

R⁴ may be hydrogen; C₁₋₈ alkyl, alkenyl, or alkynyl optionally interrupted by oxygen or sulfur; cycloalkyl; alkoxy, arylalkoxy, or heteroarylalkoxy; and

R₅, when present, may be hydrogen, halo, hydroxy, lower alkyl, lower alkenyl, cycloalkyl, C₁₋₃ alkoxy, cyano, or CF₃. n is 0, 1 or 2. In an embodiment, Y and R₃ are not both a ring.

In another aspect, the invention relates to compounds of Formula I-A:

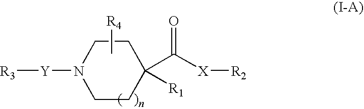

or a pharmaceutically acceptable salt thereof, wherein:

R₁ is hydrogen; alkyl, alkoxyalkyl, alkoxyphenyl, alkylthioalkyl, alkylamino, —SO₂(alkyl), —OR₈, —N(R₇)(R₈), C₃₋₆ cycloalkyl, C₃₋₆heterocycloalkyl, aryl, heteroaryl, aralkyl, or heteroaralkyl, each of which is optionally substituted with 1, 2, or 3 occurrences of R₅; or R₁ is optionally substituted (C₁-C₆alkylene)-R₁ₐ, wherein R₁ₐ is C₃₋₆ cycloalkyl, C₃₋₆heterocycloalkyl, aryl, or heteroaryl, each of which is optionally substituted with 1, 2, or 3 occurrences of R₅;

X is a direct bond, C(R₁₀)₂, NR₁₀, N(R₁₀)CO, N(R₁₀)SO₂, or

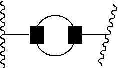

which is a 4, 5, or 6 membered ring wherein Z is N, CH, or C(C₁₋₃ alkyl); or X is NR₁₀(C₁-C₆alkylene), NR₁₀(C₁-C₆alkylene)SO₂—, or (C₁-C₆alkylene)SO₂—, wherein (C₁-C₆alkylene) is optionally substituted with 1, 2, or 3 substituents selected from the group consisting of halogen, methyl, and ethyl, or (C₁-C₆alkylene) is geminally substituted to form a cyclopropyl ring;

R₂ is cycloalkyl, aryl, heterocyclic, heteroaryl, aralkyl, heteroaralkyl, or aralkenyl; each of which is optionally substituted with 1, 2, or 3 occurrences of R₅;

Y is a direct bond, CO, SO₂, NHCO, NHSO₂, —C(═NR₁₀)—, C₁₋₄ alkylene, C₁₋₄ alkylene-SO₂—, —C(O)—C₁₋₄ alkylene, C₃₋₆ cycloalkylene, arylene, heterocycloalkylene, or heteroarylene; each of which is optionally substituted with 1, 2, or 3 occurrences of R₅;

R₃ is hydrogen or —N(alkyl)₂; or alkyl, alkenyl, alkynyl, cycloalkyl, aryl, heterocycloalkyl, or heteroaryl; each of which is optionally substituted with 1, 2, or 3 occurrences of R₅;

R₄ is hydrogen, C₁₋₈ alkyl, alkenyl, alkynyl, cycloalkyl, alkoxy, arylalkoxy, or heteroarylalkoxy;

R₅, when present, represents independently for each occurrence hydrogen, halogen, hydroxy, alkyl, alkenyl, cycloalkyl, alkoxy, —CO₂H, —CO₂C₁₋₃alkyl, —C(O)N(H)alkyl, —C(O)N(H)SO₂alkyl, —O-alkyleneCO₂R₁₀, cyano, oxo, aryl, heteroaryl, heterocyclic, alicyclic, CF₃, O—CF₃, O—CHF₂, —O-aryl, —N(alkyl)C(O)alkyl, —N(H)SO₂alkyl, C₁₋₃alkyl-S(O)₂—NH—, or C₁₋₃ alkyl-C(O)—NH—;

n is 0, 1 or 2;

R₇ is hydrogen or C₁₋₃ alkyl;

R₈ is alkyl, alicyclic, aryl, heterocyclic or heteroaryl; and

R₁₀ is hydrogen, C₁₋₂ alkyl, or C₁₋₂ alkenyl.

In certain embodiments, the compound is:
N-(3,5-Bis(trifluoromethyl)benzyl)-1-((tetrahydro-2H-pyran-4-yl)methyl)-4-methylpiperidine-4-carboxamide;
N-(3,5-Bis(trifluoromethyl)benzyl)-1-((benzo[d][1,3]dioxol-5-yl)methyl)-4-methylpiperidine-4-carboxamide;
1-(Benzo[d][1,3]dioxol-5-ylmethyl)-N-(3,4-dichlorobenzyl)-4-methylpiperidine-4-carboxamide;
N-(3,4-dichlorobenzyl)-4-methyl-1-(tetrahydro-2H-pyran-4-yl)methyl)piperidine-4-carboxamide;
1-(Benzo[c][1,2,5]oxadiazol-5-ylmethyl)-N-(3,4-dichlorobenzyl)-4-methylpiperidine-4-carboxamide;
1-(Benzo[c][1,2,5]oxadiazol-5-ylmethyl)-N-(3,4-dichlorobenzyl)-4-methylpiperidine-4-carboxamide;
1-(Benzo[c][1,2,5]oxadiazol-5-ylmethyl)-N-(3,4-dichlorobenzyl)-4-methylpiperidine-4-carboxamide;
N-(3-Fluoro-5-(trifluoromethyl)benzyl)-4-methyl-1-((tetrahydro-2H-pyran-4-yl)methyl)piperidine-4-carboxamide;
N-(3,5-Bis(trifluoromethyl)benzyl)-1-((tetrahydro-2H-pyran-4-yl)methyl)-4-(methoxymethyl)piperidine-4-carboxamide;
N-(3,5-Bis(trifluoromethyl)benzyl)-1-((tetrahydro-2H-pyran-4-yl)methyl)-4-isobutylpiperidine-4-carboxamide;
N-(3,5-Bis(trifluoromethyl)benzyl)-4-(pyridin-4-ylmethyl)-1-((tetrahydro-2H-pyran-4-yl)methyl)piperidine-4-carboxamide;
4-Benzyl-N-(3,5-bis(trifluoromethyl)benzyl)-1-((tetrahydro-2H-pyran-4-yl)methyl)piperidine-4-carboxamide;
N-(3,5-Bis(trifluoromethyl)benzyl)-4-(4-methoxybenzyl)-1-((tetrahydro-2H-pyran-4-yl)methyl)piperidine-4-carboxamide;
N-(3,5-Bis(trifluoromethyl)benzyl)-1-(tetrahydro-2H-pyran-4-yl)methyl)-4-(thiophene-3-ylmethyl)piperidine-4-carboxamide;
N-(3,5-Bis(trifluoromethyl)benzyl)-4-(pyridin-3-ylmethyl)-1-((tetrahydro-2H-pyran-4-yl)methyl)piperidine-4-carboxamide;
N-(3,5-Bis(trifluoromethyl)benzyl)-4-isobutyl-1-(2-(piperidin-1-yl)ethyl)piperidine-4-carboxamide;
N-(3,5-Bis(trifluoromethyl)benzyl)-4-isobutyl-1-(pyridin-2-yl)piperidine-4-carboxamide;
N-(3,5-Bis(trifluoromethyl)benzyl)-1-(furan-2-ylmethyl)-4-isobutylpiperidine-4-carboxamide;
1-(Benzo[d][1,3]dioxol-5-ylmethyl)-N-(3,5-bis(trifluoromethyl)benzyl)-4-isobutylpiperidine-4-carboxamide;
1-(Benzo[d][1,3]dioxol-5-ylmethyl)-N-(3,5-bis(trifluoromethyl)benzyl)-4-(pyridin-3-ylmethyl)piperidine-4-carboxamide;
1-(Benzo[d][1,3]dioxo-5-ylmethyl)-N-(3,5-bis(trifluoromethyl)benzyl)-4-(thiophene-3-ylmethyl)piperidine-4-carboxamide;
1-(Benzo[d][1,3]dioxol-5-ylmethyl)-N-(3,5-bis(trifluoromethyl)benzyl)-4-(pyridin-4-ylmethyl)piperidine-4-carboxamide;
1-(Benzo[d][1,3]dioxol-5-ylmethyl)-4-isobutyl-N-(3-(trifluoromethyl)benzyl)piperidine-4-carboxamide;
1-(4-(Benzo[d][1,3]dioxol-5-yl)-4-hydroxycyclohexyl)-N-(3,5-bis(trifluoromethyl)benzyl)-4-isobutylpiperidine-4-carboxamide
1-(4-(Benzo[d][1,3]dioxol-5-yl)-4-hydroxycyclohexyl)-N-(3,5-bis(trifluoromethyl)benzyl)-4-isobutylpiperidine-4-carboxamide;
1-(Benzo[d][1,3]dioxol-5-ylmethyl)-N-(3,5-bis(trifluoromethyl)benzyl)-4-((tetrahydro-2H-pyran-4-yl)methyl)piperidine-4-carboxamide;
1-(Benzo[d][1,3]dioxol-5-ylmethyl)-N-(3,5-bis(trifluoromethyl)benzyl)-4-(4-methoxybenzyl)piperidine-4-carboxamide;
1-(Benzo[d][1,3]dioxol-4-ylmethyl)-N-(3,5-bis(trifluoromethyl)benzyl)-4-isobutylpiperidine-4-carboxamide;
N-(3,5-Bis(trifluoromethyl)benzyl)-1-(3-hydroxy-4-methoxybenzyl)-4-isobutylpiperidine-4-carboxamide;
N-(3,5-Bis(trifluoromethyl)benzyl)-1-(4-hydroxy-3-methoxybenzyl)-4-isobutylpiperidine-4-carboxamide;
1-(Benzo[d][1,3]dioxol-5-ylmethyl)-4-benzyl-N-(3,5-bis(trifluoromethyl)benzyl)piperidine-4-carboxamide;
1-(Benzo[d][1,3]dioxol-5-ylmethyl)-N-(3-fluoro-5-(trifluoromethyl)benzyl)-4-isobutylpiperidine-4-carboxamide;
1-(Benzo[d][1,3]dioxol-5-ylmethyl)-N-(3,5-dimethoxybenzyl)-4-isobutylpiperidine-4-carboxamide;
1-(4-(2H-tetrazol-5-yl)benzyl)-N-(3,5-bis(trifluoromethyl)benzyl)-4-isobutylpiperidine-4-carboxamide;
1-(4H-Imidazol-2-yl)methyl)-N-(3,5-bis(trifluoromethyl)benzyl)-4-isobutylpiperidine-4-carboxamide;
N-(3,5-Bis(trifluoromethyl)benzyl)-4-isobutyl-1-((6-methoxypyridin-3-yl)methyl)piperidine-4-carboxamide;
N-(3,5-Bis(trifluoromethyl)benzyl)-1-(3,4-dimethoxybenzyl)-4-isobutylpiperidine-4-carboxamide;
(1-(4-Hydroxy-3-methoxybenzyl)-4-isobutylpiperidin-4-yl)(4-(3-(trifluoromethyl)phenyl)piperazin-1-yl)methanone;
(1-(Benzo[d][1,3]dioxol-5-ylmethyl)-4-isobutylpiperidin-4-yl)(4-(3-(trifluoromethyl)phenyl)piperazin-1-yl)methanone;
N-(3,5-Bis(trifluoromethyl)benzyl)-4-(cyclopropylmethyl)-1-(4-hydroxy-3-methoxybenzyl)piperidine-4-carboxamide;
1-(Benzo[d][1,3]dioxol-5-ylmethyl)-N-(3,5-bis(trifluoromethyl)benzyl)-4-(cyclopropylmethyl)piperidine-4-carboxamide;
1-(Benzo[d][1,3]dioxol-5-ylmethyl)-4-(3,5-bis(trifluoromethyl)benzyl)-N-isopropylpiperidine-4-carboxamide;
1-(Benzo[d][1,3]dioxol-5-ylmethyl)-N-(3,5-bis(trifluoromethyl)benzyl)-4-(isopropylsulfonyl)piperidine-4-carboxamide;
(R)-1-(Benzo[d][1,3]dioxol-5-ylmethyl)-N-(1-(3,5-bis(trifluoromethyl)phenyl)ethyl)-4-isobutylpiperidine-4-carboxamide;
(S)-1-(benzo[d][1,3]dioxol-5-ylmethyl)-N-(1-(3,5-bis(trifluoromethyl)phenyl)ethyl)-4-isobutylpiperidine-4-carboxamide;
N-(3,5-Bis(trifluoromethyl)benzyl)-1-(2-fluoro-4,5-dimethoxybenzyl)-4-isobutylpiperidine-4-carboxamide;
N-(3,5-Bis(trifluoromethyl)benzyl)-4-(cyclobutylmethyl)-1-(4-hydroxy-3-methoxybenzyl)piperidine-4-carboxamide;
1-(Benzo[d][1,3]dioxol-5-ylmethyl)-N-(3,5-bis(trifluoromethyl)benzyl)-4-(cyclobutylmethyl)piperidine-4-carboxamide;
N-(3,5-Bis(trifluoromethyl)benzyl)-1-(4-hydroxy-3-methoxybenzyl)-4-(oxazol-2-ylmethyl)piperidine-4-carboxamide;
N-(3,5-Bis(trifluoromethyl)benzyl)-4-(cyclopropylmethyl)-1-((2,2-difluorobenzo[d][1,3]dioxol-5-ylmethyl)piperidine-4-carboxamide;
N-(3,5-Bis(trifluoromethyl)benzyl)-1-(1-(4-hydroxy-3-methoxyphenyl)ethyl)-4-isobutylpiperidine-4-carboxamide;
1-(Benzo[d][1,3]dioxol-5-ylmethyl)-N-(3,5-dimethylbenzyl)-4-isobutylpiperidine-4-carboxamide;
N-(3,5-Bis(trifluoromethyl)benzyl)-4-isobutyl-1-(2-oxo-2-sulphonylmorpholinoethyl)piperidine-4-carboxamide;
N-(3,5-Bis(trifluoromethyl)benzyl)-4-isobutyl-1-(2-morpholino-2-oxoethyl)piperidine-4-carboxamide;
N-(3,5-Bis(trifluoromethyl)benzyl)-4-((2,2-difluorocyclopropyl)methyl)-1-(4-hydroxy-3-methoxybenzyl)piperidine-4-carboxamide;
(4-Isobutyl-1-((tetrahydro-2H-pyran-4-yl)methyl)piperidin-4-yl)(4-(3-(trifluoromethyl)phenyl)piperazin-1-yl)methanone;
N-(3,5-Bis(trifluoromethyl)benzyl)-1-(4-hydroxy-4-(6-methoxypyridin-3-yl)cyclohexyl)-4-isobutylpiperidine-4-carboxamide;
N-(3,5-Bis(trifluoromethyl)benzyl)-1-(4-hydroxy-4-(6-methoxypyridin-3-yl)cyclohexyl)-4-isobutylpiperidine-4-carboxamide;
5-((4-(3,5-Bis(trifluoromethyl)benzylcarbamoyl)-4-(cyclopropylmethyl)piperidin-1-yl)methyl)-2-fluorobenzoic acid;
N-(3,5-Bis(trifluoromethyl)benzyl)-4-(cyclopropylmethyl)-1-(4-fluoro-3-(2-(pyrrolidin-1-yl)ethylcarbamoyl)benzyl)piperidine-4-carboxamide;
N-(2-(4-(3,5-Bis(trifluoromethyl)benzylcarbamoyl)-4-isobutylpiperidin-1-yl)ethyl)-6-methoxynicotinamide;
5-((4-(3,5-Bis(trifluoromethyl)benzylcarbamoyl)-4-(cyclopropylmethyl)piperidin-1-yl)methyl)-2-methoxybenzoic acid;
4-((4-(3,5-Bis(trifluoromethyl)benzylcarbamoyl)-4-(cyclopropylmethyl)piperidin-1-yl)methyl)-2-methoxybenzoic acid;
5-((4-(3,5-Bis(trifluoromethyl)benzylcarbamoyl)-4-(cyclopropylmethyl)piperidin-1-yl)methyl)-2-hydroxybenzoic acid;
N-(3,5-Bis(trifluoromethyl)benzyl)-4-(cyclopropylmethyl)-1-(3-(ethylsulfonylcarbamoyl)-4-fluorobenzyl)piperidine-4-carboxamide;
N-(3,5-Bis(trifluoromethyl)benzyl)-4-(cyclopropylmethyl)-1-(2-oxo-2-((tetrahydrofuran-2-yl)methylamino)ethyl)piperidine-4-carboxamide;
4-((4-(3,5-Bis(trifluoromethyl)benzylcarbamoyl)-4-(cyclopropylmethyl)piperidin-1-yl)methyl)-2-fluorobenzoic acid;
N-(3,5-Bis(trifluoromethyl)benzyl)-1-((6-(4-cyanophenoxy)pyridin-3-yl)methyl)-4-(cyclopropylmethyl)piperidine-4-carboxamide;
2-(2-((4-(3,5-Bis(trifluoromethyl)benzylcarbamoyl)-4-(cyclopropylmethyl)piperidin-1-yl)methyl)-6-methoxyphenoxy)acetic acid;
N-(3,5-Bis(trifluoromethyl)benzyl)-4-(cyclopropylmethyl)-1-(2-methyl-2-morpholinopropyl)piperidine-4-carboxamide;
N-(3,5-Bis(trifluoromethyl)benzyl)-4-(cyclopropylmethyl)-1-((2-morpholinothiazol-5-yl)methyl)piperidine-4-carboxamide;
1-(3-(1H-imidazol-1-yl)benzyl-N-(3,5-bis(trifluoromethyl)benzyl)-4-(cyclopropylmethyl)piperidine-4-carboxamide;
N-(3,5-Bis(trifluoromethyl)benzyl)-1-((5-(4-chlorophenyl)isoxazol-3-yl)methyl)-4-(cyclopropylmethyl)piperidine-4-carboxamide;
N-(3,5-Bis(trifluoromethyl)benzyl)-4-(cyclopropylmethyl)-1-(3-((1,3-dimethyl-1H-pyrazol-5-yl)methoxy)-4-methoxybenzyl)piperidine-4-carboxamide;
N-(3,5-Bis(trifluoromethyl)benzyl)-4-(cyclopropylmethyl)-1-((6-(thiophen-2-yl)pyridin-3-yl)methyl)piperidine-4-carboxamide;
N-(3,5-Bis(trifluoromethyl)benzyl)-4-(cyclopropylmethyl)-1-((1-(thiazol-2-yl)-1H-pyrrol-2-yl)methyl)piperidine-4-carboxamide;
N-(3,5-Bis(trifluoromethyl)benzyl)-4-(cyclopropylmethyl)-1-((3-phenylisoxazol-5-yl)piperidine-4-carboxamide;
N-(3,5-Bis(trifluoromethyl)benzyl)-4-(cyclopropylmethyl)piperidine-4-carboxamide;
N-(3,5-Bis(trifluoromethyl)benzyl)-4-(cyclopropylmethyl)-1-(3-fluoro-4-(2-(pyrrolidin-1-yl)ethylcarbamoyl)benzyl)piperidine-4-carboxamide;
N-(3,5-Bis(trifluoromethyl)benzyl)-3-(cyclopropylmethyl)pyrrolidine-3-carboxamide;
(rac)N-(3,5-Bis(trifluoromethyl)benzyl)-3-(cyclopropylmethyl)-1-(4-hydroxy-3-methoxybenzyl)pyrrolidine-3-carboxamide;
N-(3,5-Bis(trifluoromethyl)benzyl)-4-(cyclopropylmethyl)-1-(2,2-dimethyl-3-oxo-3-(thiazol-2-ylamino)propyl)piperidine-4-carboxamide;
5-((3-(3,5-Bis(trifluoromethyl)benzylcarbamoyl)-3-(cyclopropylmethyl)pyrrolidin-1-yl)methyl)-2-fluorobenzoic acid;
N-(3,5-Bis(trifluoromethyl)benzyl)-3-(cyclopropylmethyl)-1-(3-fluoro-4-methoxybenzyl)pyrrolidine-3-carboxamide;
N-(3,5-Bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-(3-fluoro-4-hydroxybenzyl)pyrrolidine-3-carboxamide;
N-(3,5-bis(trifluoromethyl)benzyl)-3-(cyclopropylmethyl)-1-(3-fluoro-4-(methylsulfonyl)benzyl)pyrrolidine-3-carboxamide;
N-(3,5-bis(trifluoromethyl)benzyl)-3-(cyclopropylmethyl)-1-((2,2-dimethylchroman-7-yl)methyl)pyrrolidine-3-carboxamide;
3-(cyclopropylmethyl)-1-((5-methoxy-2-methyl-2,3-dihydrobenzofuran-6-yl)methyl)-N-((5-(trifluoromethyl)pyridin-3-yl)methyl)pyrrolidine-3-carboxamide;
N-(3,5-bis(trifluoromethyl)benzyl)-3-(cyclopropylmethyl)-1-((5-methoxy-2-methyl-2,3-dihydrobenzofuran-6-yl)methyl)pyrrolidine-3-carboxamide or a pharmaceutically acceptable salt thereof.

What is also provided is a compound which is:
N-(3,5-bis(trifluoromethyl)benzyl)-3-(cyclopropylmethyl)-1-(3-(ethylsulfonylcarbamoyl)-4-fluorobenzyl)pyrrolidine-3-carboxamide;
N-(3,5-bis(trifluoromethyl)benzyl)-3-(cyclopropylmethyl)-1-(4-fluoro-3-(methylsulfonamido)benzyl)pyrrolidine-3-carboxamide;
5-((3-(3,5-bis(trifluoromethyl)benzylcarbamoyl)-3-(cyclopropylmethyl)pyrrolidin-1-yl)methyl)-2-fluorobenzoic acid;
N-(3,5-bis(trifluoromethyl)benzyl)-3-(cyclopropylmethyl)-1-(3-fluoro-4-(methylsulfonamido)benzyl)pyrrolidine-3-carboxamide;
N-(3,5-bis(trifluoromethyl)benzyl)-3-(cyclopropylmethyl)-1-(3-fluoro-4-methoxybenzyl)pyrrolidine-3-carboxamide;
N-(3,5-bis(trifluoromethyl)benzyl)-3-(cyclopropylmethyl)-1-(4-fluoro-3-(N-methylacetamido)benzyl)pyrrolidine-3-carboxamide;
N-(3,5-bis(trifluoromethyl)benzyl)-3-(cyclopropylmethyl)-1-(4-fluoro-3-methoxybenzyl)pyrrolidine-3-carboxamide;
N-(3,5-bis(trifluoromethyl)benzyl)-3-(cyclopropylmethyl)-1-((2,2-difluorobenzo[d][1,3]dioxol-5-yl)methyl)pyrrolidine-3-carboxamide;
N-(3,5-bis(trifluoromethyl)benzyl)-3-(cyclopropylmethyl)-1-(4-fluoro-3-hydroxybenzyl)pyrrolidine-3-carboxamide;
N-(3,5-bis(trifluoromethyl)benzyl)-3-(cyclopropylmethyl)-1-((3-methyloxetan-3-yl)methyl)pyrrolidine-3-carboxamide;
N-(3,5-bis(trifluoromethyl)benzyl)-3-(cyclopropylmethyl)-1-(2-methyl-2-morpholinopropyl)pyrrolidine-3-carboxamide;
N-(3,5-bis(trifluoromethyl)benzyl)-3-(cyclopropylmethyl)-1-((4′-fluorobiphenyl-4-yl)methyl)pyrrolidine-3-carboxamide;
N-(3,5-bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((3,5-dimethyltetrahydro-2H-pyran-4-yl)methyl)pyrrolidine-3-carboxamide;
N-(3,5-bis(trifluoromethyl)benzyl)-3-(cyclopropylmethyl)-1-(3-methoxytetrahydro-2H-pyran-4-yl)pyrrolidine-3-carboxamide;
N-(3,5-bis(trifluoromethyl)benzyl)-3-(cyclopropylmethyl)-1-((4,4-difluorocyclohexyl)methyl)pyrrolidine-3-carboxamide;
N-(3,5-bis(trifluoromethyl)benzyl)-3-(cyclopropylmethyl)-1-(4-hydroxy-4-(6-methoxypyridin-3-yl)cyclohexyl)pyrrolidine-3-carboxamide;
1-(4-(benzo[d][1,3]dioxol-5-yl)-4-hydroxycyclohexyl)-N-(3,5-bis(trifluoromethyl)benzyl)-3-(cyclopropylmethyl)pyrrolidine-3-carboxamide;
N-(3,5-bis(trifluoromethyl)benzyl)-4-(cyclopropylmethyl)-1-(4-hydroxy-2-methoxycyclohexyl)piperidine-4-carboxamide;
N-(3,5-bis(trifluoromethyl)benzyl)-3-(cyclopropylmethyl)-1-(4-hydroxy-2-methoxycyclohexyl)pyrrolidine-3-carboxamide;

(S)—N-(3,5-bis(trifluoromethyl)benzyl)-1-(4-hydroxy-3-methoxybenzyl)pyrrolidine-3-carboxamide;
(R)—N-(3,5-bis(trifluoromethyl)benzyl)-1-(4-hydroxy-3-methoxybenzyl)pyrrolidine-3-carboxamide;
N-(3,5-bis(trifluoromethyl)benzyl)-3-(cyclopropylmethyl)-1-((5-fluoro-1-methyl-6-oxo-1,6-dihydropyridin-3-yl)methyl)pyrrolidine-3-carboxamide;
N-(3,5-bis(trifluoromethyl)benzyl)-3-(cyclopropylmethyl)-1-((2-methoxypyridin-4-yl)methyl)pyrrolidine-3-carboxamide;
(3-(cyclopropylmethyl)-1-(4-hydroxy-3-methoxybenzyl)pyrrolidin-3-yl)(4-(4-(trifluoromethyl)pyridin-2-yl)piperazin-1-yl)methanone;
(3-(cyclopropylmethyl)-1-(4-hydroxy-3-methoxybenzyl)pyrrolidin-3-yl)(4-(3-(trifluoromethyl)phenyl)piperazin-1-yl)methanone;
N-(3,5-bis(trifluoromethyl)benzyl)-1-(4-hydroxy-3-methoxybenzyl)-3-((tetrahydro-2H-pyran-4-yl)methyl)pyrrolidine-3-carboxamide;
N-(3,5-bis(trifluoromethyl)benzyl)-1-(4-hydroxy-3-methoxybenzyl)-3-(methoxymethyl)pyrrolidine-3-carboxamide;
N-(3,5-bis(trifluoromethyl)benzyl)-1-(4-hydroxy-3-methoxybenzyl)-3-isobutylpyrrolidine-3-carboxamide;
N-(3,5-bis(trifluoromethyl)benzyl)-1-(4-hydroxy-3-methoxybenzyl)-3-(2-methoxyethyl)pyrrolidine-3-carboxamide;
5-((3-(3,5-bis(trifluoromethyl)benzylcarbamoyl)-3-(cyclopropylmethyl)pyrrolidin-1-yl)methyl)-2-fluorobenzoic acid;
N-(3,5-bis(trifluoromethyl)benzyl)-3-(cyclopropylmethyl)-1-(3-fluoro-4-methoxybenzyl)pyrrolidine-3-carboxamide;
N-(3,5-bis(trifluoromethyl)benzyl)-3-(cyclopropylmethyl)-1-(4-fluoro-3-methoxybenzyl)pyrrolidine-3-carboxamide;
N-(3,5-bis(trifluoromethyl)benzyl)-3-(cyclopropylmethyl)-1-((tetrahydrofuran-3-yl)methyl)pyrrolidine-3-carboxamide;
1-(1H-imidazol-1-yl)benzyl)-N-(3,5-bis(trifluoromethyl)benzyl)-3-(cyclopropylmethyl)pyrrolidine-3-carboxamide;
N-(3,5-bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((tetrahydro-2H-pyran-3-yl)methyl)pyrrolidine-3-carboxamide;
N-(3,5-bis(trifluoromethyl)benzyl)-4-(cyclopropylmethyl)-1-(4-hydroxy-2-methoxycyclohexyl)piperidine-4-carboxamide;
N-(3,5-bis(trifluoromethyl)benzyl)-3-(cyclopropylmethyl)-1-(4-hydroxy-2-methoxycyclohexyl)pyrrolidine-3-carboxamide;
(S)—N-(3,5-bis(trifluoromethyl)benzyl)-1-(4-hydroxy-3-methoxybenzyl)pyrrolidine-3-carboxamide;
(R)—N-(3,5-bis(trifluoromethyl)benzyl)-1-(4-hydroxy-3-methoxybenzyl)pyrrolidine-3-carboxamide;
N-(3,5-bis(trifluoromethyl)benzyl)-3-(cyclopropylmethyl)-1-(3-fluoro-4-hydroxybenzyl)pyrrolidine-3-carboxamide;
N-(3,5-bis(trifluoromethyl)benzyl)-3-(cyclopropylmethyl)-1-(3-fluoro-4-(methyl sulfonyl)benzyl)pyrrolidine-3-carboxamide;
N-(3,5-bis(trifluoromethyl)benzyl)-3-(cyclopropylmethyl)-1-((4-methoxycyclohexyl)methyl)pyrrolidine-3-carboxamide;
N-(3,5-bis(trifluoromethyl)benzyl)-3-(cyclopropylmethyl)-1-((3-methoxytetrahydro-2H-pyran-4-yl)methyl)pyrrolidine-3-carboxamide;
N-(3,5-bis(trifluoromethyl)benzyl)-3-isopentyl-1-((tetrahydro-2H-pyran-4-yl)methyl)pyrrolidine-3-carboxamide;
N-(3,5-bis(trifluoromethyl)benzyl)-3-((2,2-difluorocyclopropyl)methyl)-1-((tetrahydro-2H-pyran-4-yl)methyl)pyrrolidine-3-carboxamide;
3-(cyclopropylmethyl)-1-((tetrahydro-2H-pyran-4-yl)methyl)-N-((5-(trifluoromethyl)pyridin-3-yl)methyl)pyrrolidine-3-carboxamide;
3-(cyclopropylmethyl)-1-((tetrahydro-2H-pyran-4-yl)methyl)-N-(1-(5-(trifluoromethyl)pyridin-3-yl)ethyl)pyrrolidine-3-carboxamide;
N-(3-cyano-5-(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((4-hydroxy-4-methylcyclohexyl)methyl)pyrrolidine-3-carboxamide;
3-benzyl-N-(3,5-bis(trifluoromethyl)benzyl)-1-((tetrahydro-2H-pyran-4-yl)methyl)pyrrolidine-3-carboxamide;
(3-(cyclopropylmethyl)-1-((tetrahydro-2H-pyran-4-yl)methyl)pyrrolidin-3-yl)(4-(4-(trifluoromethyl)pyridin-2-yl)piperazin-1-yl)methanone;
(3-(cyclopropylmethyl)-1-((tetrahydro-2H-pyran-4-yl)methyl)pyrrolidin-3-yl)(3-(4-(trifluoromethyl)pyridin-2-yl)azetidin-1-yl)methanone;
1-(4-acetamido-3-fluorobenzyl)-3-(cyclopropylmethyl)-N-((5-(trifluoromethyl)pyridin-3-yl)methyl)pyrrolidine-3-carboxamide;
N-(3,5-bis(trifluoromethyl)benzyl)-3-(oxetan-3-ylmethyl)-1-((tetrahydro-2H-pyran-4-yl)methyl)pyrrolidine-3-carboxamide;
N-(3,5-bis(trifluoromethyl)benzyl)-3-(cyclobutylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((2,2-dimethyltetrahydro-2H-pyran-4-yl)methyl)pyrrolidine-3-carboxamide;
N-(3,5-bis(trifluoromethyl)benzyl)-3-(cyclopropylmethyl)-1-((3-methoxytetrahydrofuran-3-yl)methyl)pyrrolidine-3-carboxamide;
1-(3-acetamido-4-fluorobenzyl)-N-(3,5-bis(trifluoromethyl)benzyl)-3-(cyclopropylmethyl)pyrrolidine-3-carboxamide;
1-(4-acetamido-3-fluorobenzyl)-N-(3,5-bis(trifluoromethyl)benzyl)-3-(cyclopropylmethyl)pyrrolidine-3-carboxamide;
(3-(cyclopropylmethyl)-1-((tetrahydro-2H-pyran-4-yl)methyl)pyrrolidin-3-yl)(4-(3-(trifluoromethyl)phenyl)piperazin-1-yl)methanone;
N-(3,5-bis(trifluoromethyl)benzyl)-3-(cyclopropylmethyl)-1-(2-methoxy-2-(methoxymethyl)butyl)pyrrolidine-3-carboxamide;
N-(3,5-bis(trifluoromethyl)benzyl)-3-(cyclopropylmethyl)-1-((4-hydroxytetrahydro-2H-pyran-4-yl)methyl)pyrrolidine-3-carboxamide;
N-(3,5-bis(trifluoromethyl)benzyl)-3-(cyclopropylmethyl)-1-((4-methoxytetrahydro-2H-pyran-4-yl)methyl)pyrrolidine-3-carboxamide;
N-(3,5-bis(trifluoromethyl)benzyl)-4-(cyclopropylmethyl)-1-((2-oxo-2H-chromen-7-yl)methyl)piperidine-4-carboxamide;

or a pharmaceutically acceptable salt thereof.

The invention also provides pharmaceutical compositions comprising compounds selected from the group of Formula I, and the use of these compounds and compositions in the prevention or treatment of diseases in which CCR2 chemokine receptors are involved.

The invention additionally provides a method for the treatment of inflammation, rheumatoid arthritis, lupus, systemic lupus erythematosus, atherosclerosis, restenosis, immune disorders, and transplant rejection in a mammal in need thereof, comprising administering to such mammal a therapeutically effective amount of a pharmaceutical composition containing a compound according to formula I in admixture with a pharmaceutically acceptable excipient, diluent, or carrier.

The invention further provides compositions comprising a compound of the invention and a pharmaceutically acceptable carrier.

The invention further provides methods of modulating the activity of a chemokine receptor comprising exposing said chemokine receptor with a compound of the invention.

The invention further provides methods of treating a disease associated with expression or activity of a chemokine receptor in a patient comprising administering to the patient a therapeutically effective amount of a compound of the invention.

The invention further provides a compound of Formula I for use in therapy.

The invention further provides use of a compound of Formula I for the manufacture of a medicament for the treatment of disease associated with expression or activity of a chemokine receptor.

The invention further provides use of a compound of the invention for preparing a medicament for treating or preventing organ transplant rejection, rheumatoid arthritis, chronic contact dermatitis, inflammatory bowel disease, lupus, systemic lupus erythematosus, multiple sclerosis, atherosclerosis, psoriasis, sarcoidosis, idiopathic pulmonary fibrosis, dermatomyositis, skin pemphigoid and related diseases, glomerulonephritides, vasculitides, hepatitis, allograft rejection, graft-versus host disease, athersclerosis, metabolic syndrome, diabetes, or obesity.

More specifically, the invention relates to new anti-inflammatory and immunomodulatory compounds and pharmaceutical compositions thereof that act via antagonism of the CCR2 receptor, therefore leading to MCP-I inhibition. The invention further relates to novel compounds for use in the compositions, to processes for their preparation, to intermediates useful in their preparation and to their use as therapeutic agents.

The chemokine receptor modulators/antagonists of the invention may be effective as therapeutic agents and/or preventive agents for diseases such as atherosclerosis, asthma, pulmonary fibrosis, myocarditis, ulcerative colitis, psoriasis, asthma, ulcerative colitis, nephritis (nephropathy), multiple sclerosis, lupus, systemic lupus erythematosus, hepatitis, pancreatitis, sarcoidosis, organ transplantation, Crohn's disease, endometriosis, congestive heart failure, viral meningitis, cerebral infarction, neuropathy, Kawasaki disease, and sepsis in which tissue infiltration of blood leukocytes, such as monocytes and lymphocytes, play a major role in the initiation, progression or maintenance of the disease.

### DETAILED DESCRIPTION OF THE INVENTION

The features and other details of the invention will now be more particularly described. It will be understood that particular embodiments described herein are shown by way of illustration and not as limitations of the invention. The principal features of this invention can be employed in various embodiments without departing from the scope of the invention. All parts and percentages are by weight unless otherwise specified.

### DEFINITIONS

For convenience, certain terms used in the specification, examples, and appended claims are collected here.

“CCR2 receptor modulator” or “CCR2 modulator” includes compounds having effect at the CCR2 receptors, including those compounds having a modulating effect primarily at CCR2.

“Treating”, includes any effect, e.g., lessening, reducing, modulating, or eliminating, that results in the improvement of the condition, disease, disorder and the like.

The symbol “ [symbol] ” indicates a point of attachment.

“Alkyl” includes saturated aliphatic groups, e.g., straight-chain alkyl groups such as methyl, ethyl, propyl, butyl, pentyl, hexyl, heptyl, octyl, nonyl, and decyl; branched-chain alkyl groups (e.g., isopropyl, tert-butyl, and isobutyl); cycloalkyl (alicyclic) groups like cyclopropyl, cyclopentyl, cyclohexyl, cycloheptyl, and cyclooctyl); lower alkyl-substituted cycloalkyl groups; and cycloalkyl-substituted alkyl groups. In an embodiment, alicyclic rings do not include bridged rings.

“Alkyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound.

Straight or branched alkyl groups may have six or fewer carbon atoms in their backbone (e.g., C₁-C₆ for straight chain, C₃-C₆ for branched chain), and more preferably four or fewer. Preferred cycloalkyl groups have from three to eight carbon atoms in their ring structure, and more preferably five or six carbons in the ring structure. “C₁-C₆” includes alkyl groups containing one to six carbon atoms.

“Substituted alkyls” refers to alkyl moieties having substituents replacing a hydrogen on one or more carbons of the hydrocarbon backbone. Such substituents can include alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, thiocarboxylate, sulfates, alkylsulfinyl, sulfonato, sulfamoyl, sulfonamido, nitro, trifluoromethyl, cyano, azido, or heterocyclyl.

“Alkylene,” unless indicated otherwise, means a straight or branched, saturated or unsaturated, aliphatic, divalent radical having the number of carbon atoms indicated (e.g., (C₁₋₆)alkylene includes methylene (—CH₂—), ethylene (—CH₂CH₂—), trimethylene (—CH₂CH₂CH₂—), and the like. Alkylene may be optionally substituted as provided for alkyl, or as otherwise indicated.

“Cycloalkylene,” unless indicated otherwise, means a cyclic, saturated or unsaturated, aliphatic, divalent radical. Cycloalkylene may be optionally substituted as described for alkyl, or as otherwise indicated. “Hetereocycloalkylene,” unless indicated otherwise, means a cyclic, saturated or unsaturated, aliphatic, divalent radical having at least 1 ring carbon atom replaced by a heteroatom. Heterocycloalkylene may be optionally substituted as described for alkyl, or as otherwise indicated.

“Aryl” includes groups with aromaticity, including 5- and 6-membered unconjugated (i.e., single-ring) aromatic groups that may include from zero to four heteroatoms, as well as conjugated (i.e., multicyclic) systems having at least one ring that is aromatic. Examples of aryl groups include benzene, phenyl, tolyl and the like. Multicyclic aryl groups include tricyclic and bicyclic systems, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, indolizine, tetralin, and methylenedioxyphenyl.

Aryl groups having heteroatoms in the ring structure may also be referred to as “aryl heterocycles”, “heterocycles,” “heteroaryls” or “heteroaromatics”; e.g., pyrrole, furan, thiophene, thiazole, isothiazole, imidazole, triazole, tetrazole, pyrazole, oxazole, isooxazole, pyridine, pyrazine, pyridazine, and pyrimidine. The aromatic ring can be substituted at one or more ring positions with, for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Arylene”, unless indicated otherwise, means an aromatic, divalent radical. The aromatic group includes 5- and 6-membered unconjugated (i.e., single-ring) aromatic moieties that may include from zero to four heteroatoms, as well as conjugated (i.e., multicyclic) systems having at least one ring that is aromatic. Arylene may be optionally substituted as described for aryl, or as otherwise indicated.

“Heteroarylene”, unless indicated otherwise, means an heteroaromatic, divalent radical. Heteroarylene may be optionally substituted as described for aryl, or as otherwise indicated.

An “alkylaryl” or an “aralkyl” moiety is an alkyl substituted with an aryl group (e.g., phenylmethyl (benzyl)).

An “alkylheteroaryl” or an “heteroaralkyl” moiety is an alkyl substituted with a heteroaryl group (e.g., phenylmethyl (benzyl)).

An “aralkenyl” moiety is an alkenyl substituted with an aryl group (e.g., —CH═CH-phenyl).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, and decenyl), branched-chain alkenyl groups, cycloalkenyl groups such as cyclopropenyl, cyclopentenyl, cyclohexenyl, cycloheptenyl, and cyclooctenyl; alkyl or alkenyl-substituted cycloalkenyl groups, and cycloalkyl or cycloalkenyl-substituted alkenyl groups.

“Alkenyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound.

Straight or branched alkenyl groups may have six or fewer carbon atoms in their backbone (e.g., C₂-C₆ for straight chain, C₃-C₆ for branched chain.) Preferred cycloalkenyl groups have from three to eight carbon atoms in their ring structure, and more preferably have five or six carbons in the ring structure. The term “C₂-C₆” includes alkenyl groups containing two to six carbon atoms.

“Substituted alkenyls” refers to alkenyl moieties having substituents replacing a hydrogen on one or more hydrocarbon backbone carbon atoms. Such substituents can include alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl.

“Alkenylene” refers to a divalent radical of unsaturated aliphatic groups analogous in length and possible substitution to the alkyls described above, and which contains at least one double bond.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups.

“Alkynyl” groups may also optionally include heteroatoms, i.e., where oxygen, nitrogen, sulfur or phosphorous atoms replaces one or more hydrocarbon backbone carbon atoms, particularly where the substitution does not adversely impact the efficacy of the resulting compound

Straight or branched chain alkynyls group may have six or fewer carbon atoms in their backbone (e.g., C₂-C₆ for straight chain, C₃-C₆ for branched chain). The term “C₂-C₆” includes alkynyl groups containing two to six carbon atoms.

“Substituted alkynyls” refers to alkynyl moieties having substituents replacing a hydrogen on one or more hydrocarbon backbone carbon atoms. Such substituents can include alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl.

“Alkynylene” refers to a divalent radical of unsaturated aliphatic groups analogous in length and possible substitution to the alkyls described above, and which contains at least one triple bond.

Unless the number of carbons is otherwise specified, “lower alkyl” includes an alkyl group, as defined above, but having from one to ten, more preferably from one to six, carbon atoms in its backbone structure. “Lower alkenyl” and “lower alkynyl” have corresponding chain lengths, e.g., 2-5 carbon atoms.

“Acyl” includes compounds and moieties which contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups. “Alkylamino” includes moieties wherein an alkyl moiety is bonded to an amino group; “dialkylamino”, “arylamino”, “diarylamino”, and “alkylarylamino” are analogously named. In some embodiments, “amino” may include acylamino and/or alkylamino groups.

“Alkoxyalkyl” includes moieties where an alkoxy group is bonded to an alkyl group; “alkoxyaryl”, “thioalkoxyalkyl”, “alkylaminoalkyl” and “alkylthioalkyl” are analogously named.

“Alkoxy” includes alkyl, alkenyl, and alkynyl groups covalently linked to an oxygen atom. Examples of alkoxy groups include methoxy, ethoxy, isopropyloxy, propoxy, butoxy, and pentoxy groups. Examples of “substituted alkoxy” groups include halogenated alkoxy groups. Substituted alkoxy groups can include alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, or heterocyclyl substituents. Examples of halogen-substituted alkoxy groups include fluoromethoxy, difluoromethoxy, trifluoromethoxy, chloromethoxy, dichloromethoxy, and trichloromethoxy.

“Oxo” refers to a “O═” group. For example, cyclohexane substituted with an oxo group is cyclohexanone.

The terms “heterocycloalkyl”, “heterocyclyl” or “heterocyclic group” include closed ring structures, e.g., 3- to 10-, or 4- to 7-membered rings which include one or more heteroatoms. Heterocyclyl groups can be saturated or unsaturated and include pyrrolidine, oxolane, thiolane, piperidine, piperizine, morpholine, lactones, lactams such as azetidinones and pyrrolidinones, sultams, sultones, and the like. Heterocyclic groups can have aromatic character such as pyrrole and furan. Heterocyclic groups includes fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like. Heterocyclic groups also include spirocyclic groups.

Heterocyclic rings may be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In an embodiment, heterocyclic rings do not include bridged rings.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or heteroatoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

“Heteroatom” includes atoms of any element other than carbon or hydrogen. Examples of heteroatoms include nitrogen, oxygen, sulfur and phosphorus.

“At least partially aromatic bicyclic ring system”, means a bicyclic ring system where either or both of the rings forming the bicycle are aromatic.

It will be noted that the structure of some of the compounds of the invention includes asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

“Contacting” refers to the bringing together of indicated moieties in an in vitro or in vivo system. For example, “contacting” a chemokine receptor with a compound of the invention includes the administration of a compound of the invention to an individual or patient, such as a human, having a chemokine receptor, as well as, for example, introducing a compound of the invention into a sample containing a cellular or purified preparation containing the chemokine receptor.

“Selective” means that a compound binds to or inhibits a chemokine receptor with greater affinity or potency, respectively, compared to at least one other chemokine receptor, or preferably compared to all other chemokine receptors of the same class (e.g., all the CC-type receptors). In some embodiments, the compounds of the invention have binding or inhibition selectivity for CCR2 over any other chemokine receptor. Selectivity can be at least about 10-fold, at least about 20-fold, at least about 50-fold, at least about 100-fold, at least about 200-fold, at least about 500-fold or at least about 1000-fold. Binding affinity and inhibitor potency can be measured according to routine methods in the art.

An “anionic group,” as used herein, refers to a group that is negatively charged at physiological pH. Preferred anionic groups include carboxylate, sulfate, sulfonate, sulfinate, sulfamate, tetrazolyl, phosphate, phosphonate, phosphinate, or phosphorothioate or functional equivalents thereof “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23).

Compounds of the Invention

The invention is directed to chemokine receptor modulators, e.g., antagonists, and their use as medicinal agents, as embodied by a compound of formula I.

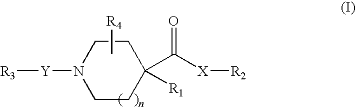

In an embodiment, R₁ may be

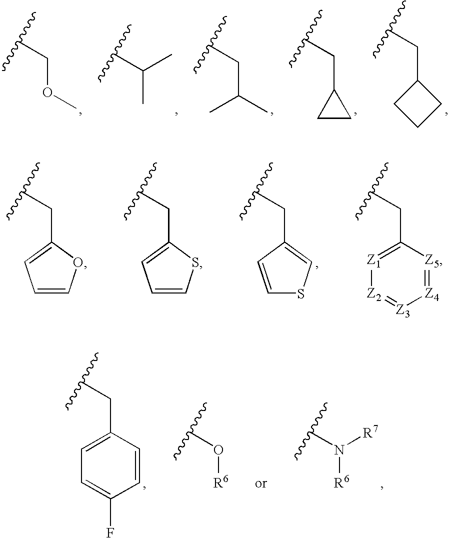

where Z₁, Z₂, Z₃, Z₄ or Z₅ are independently N, CH, or CR⁵, provided that there are no more than three Z moieties with a non-hydrogen R₅; R⁶ is hydrogen, alkyl, cycloalkyl, heterocycloalkyl, aryl, heteroaryl, or SO₂R⁸; R⁷ is hydrogen or C₁₋₃ alkyl; and R⁸ is an alkyl, alicyclic, aromatic, heterocyclic or heteroaromatic group. More desirably R¹ may be hydrogen; unsubstituted alkyl, alkoxyalkyl, alkoxyphenyl, or alkylthioalkyl; a C₃₋₆ alicyclic or heterocyclic ring; or a heteroaryl ring which, when substituted, has no more than three R⁵ substituents. Even more desirably R¹ may be unsubstituted alkyl or alkoxyalkyl.

In an embodiment, R² may be

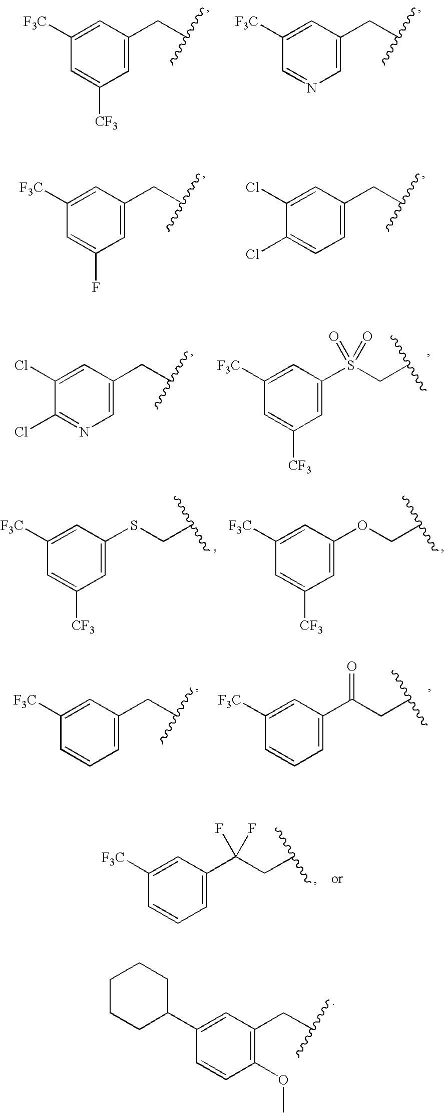

More desirably R² may be a non-fused alicyclic, aromatic, heterocyclic or heteroaromatic ring; which ring, when substituted, has no more than three R⁵ substituents. Even more desirably R² may be an aromatic ring, which, when substituted, has no more than three R⁵ halo or mono-, di- or trihaloalkyl substituents.

In an embodiment, X may be a direct bond and R² may be

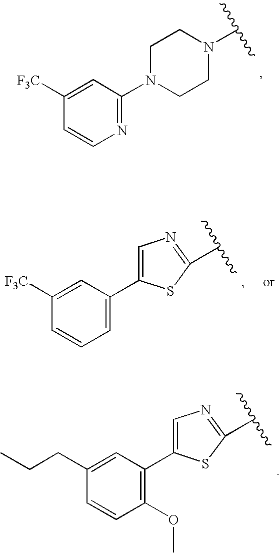

More desirably X may be NH or NHCH₂.

In an embodiment, Y is

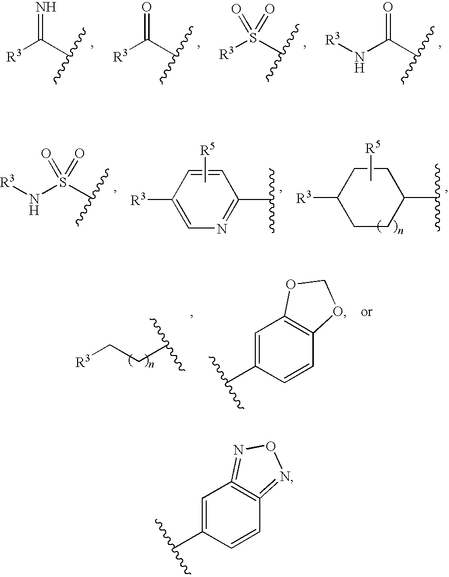

where n is 0, 1 or 2. More desirably Y may be a C₁, C₂, C₃ or C₄ alkylene group.

In an embodiment, R³ may be

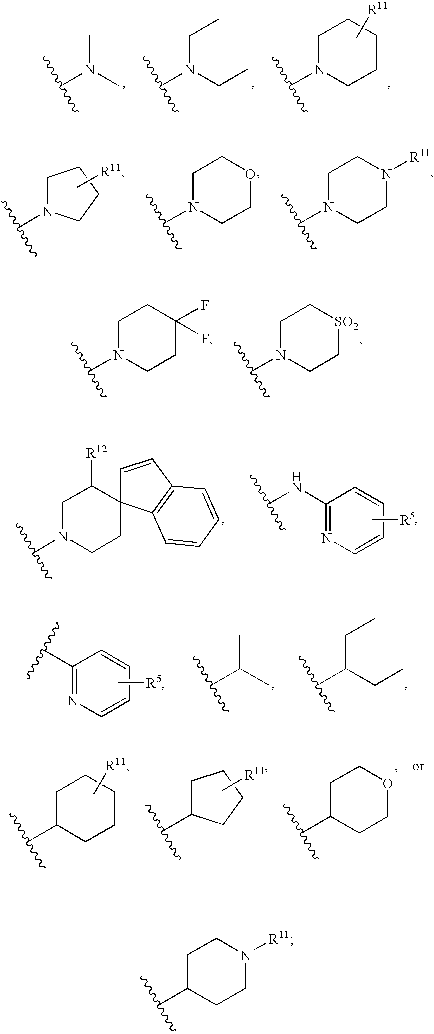

wherein R¹¹ is hydrogen; lower alkyl; hydroxy; amino; alkoxy; SO₂-lower alkyl; or an unsubstituted alicyclic, aromatic, heterocyclic or heteroaromatic ring; and R¹² is hydrogen or C₁₋₃ alkyl.

In another aspect, the invention provides a compound of formula I-A:

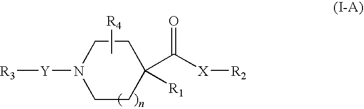

A specific value for R₁ is hydrogen. Another specific value for R₁ is alkyl. Another specific value for R₁ is alkoxyalkyl. Other specific values for R₁ are C₃₋₆ cycloalkyl, C₃₋₆ heterocycloalkyl, aryl, heteroaryl, and (C₁-C₆alkylene)-R₁ₐ, wherein R₁ₐ is C₃₋₆ cycloalkyl, C₃₋₆ heterocycloalkyl, aryl, or heteroaryl. Each of the above values is optionally substituted with 1, 2, or 3 occurrences of R₅.

Another specific value for R₁ is

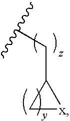

wherein

z is 1, 2, or 3;

y is 1, 2, 3, or 4;

x is O, NH, CH₂, CF₂, or N—C₁₋₈alkyl.

Other specific values for R₁ are methyl;

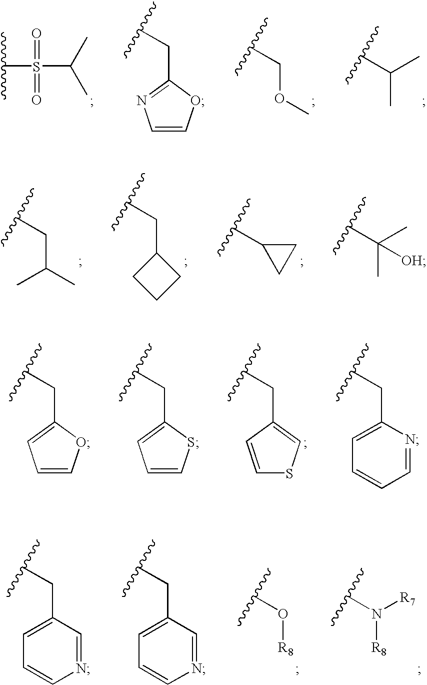

any of which is optionally substituted on carbon with 1, 2, or 3 fluorine atoms, and wherein:

R₇ is hydrogen or C₁₋₃ alkyl; and

R₈ is alkyl, alicyclic, aryl, heterocyclic or heteroaryl.

A specific value for R₂ is cycloalkyl. Another specific value for R₂ is aryl. Other specific values for R₂ are heterocycloalkyl or heteroaryl; each of which is optionally substituted with 1, 2, or 3 occurrences of R₅.

Other specific values for R₂ include

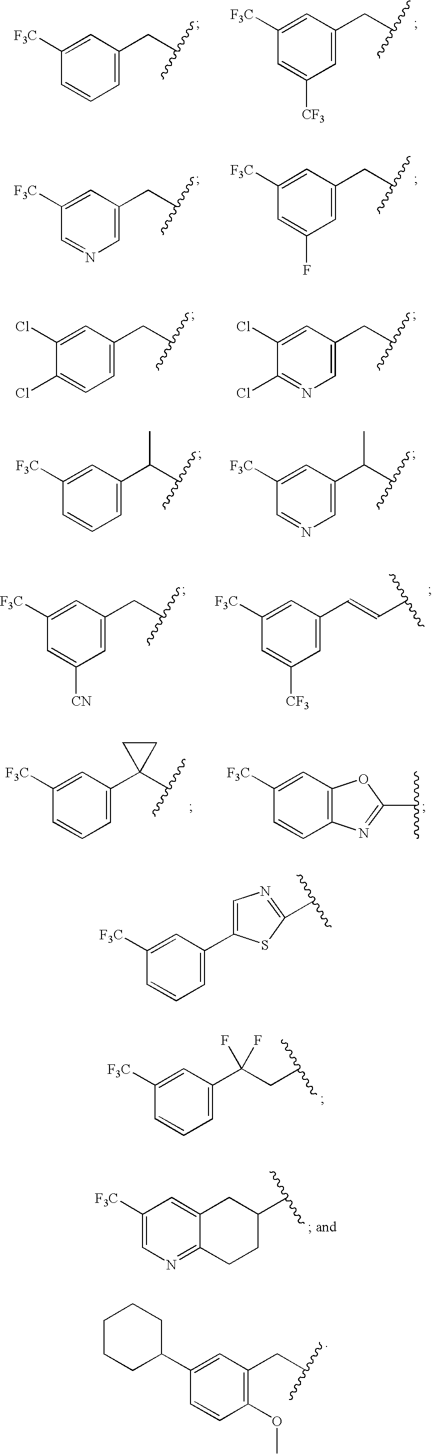

A specific value for X is a direct bond. Another specific value for X is NH. Another specific value for X is N-methyl. Other specific values for X include N-ethyl; NHCH₂; N(methyl)-CH₂; N(ethyl)-CH₂; CH₂; CH(methyl); CH(ethyl); NHCO; and NHSO₂.

A specific value for X—R₂ is

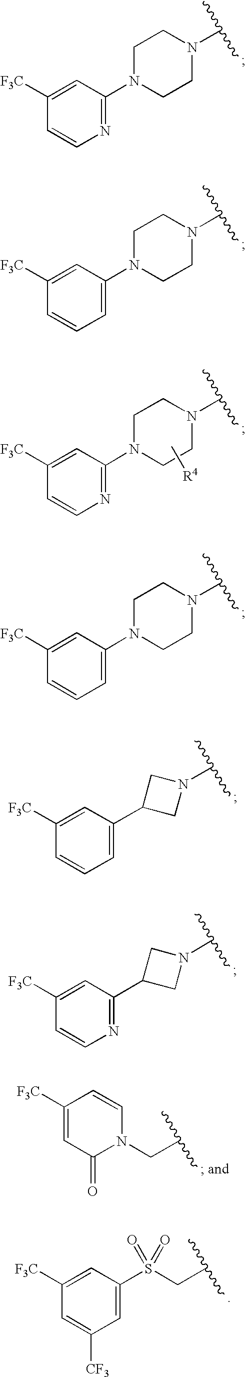

A specific value for Y is

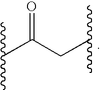

Another specific value for Y is

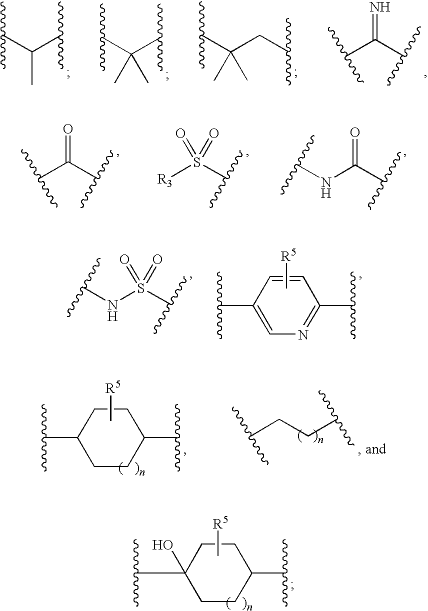

wherein n is 0, 1 or 2.

A specifc value for R₃ is

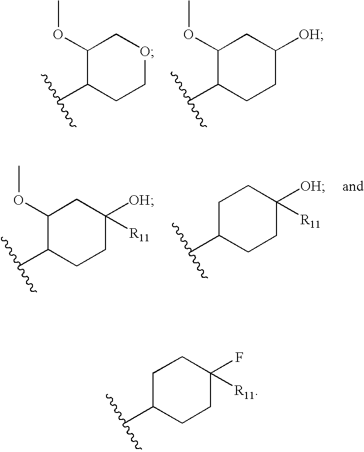

Other specific values for R₃ include

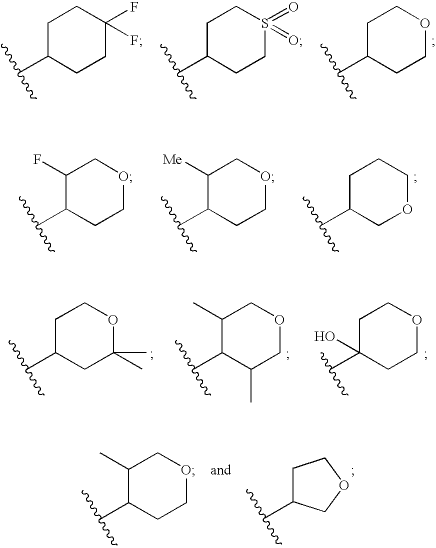

wherein R₁₁ is selected from the group consisting of an substituted or unsubstituted alicyclic, aryl, heterocyclic, or heteroaryl.

Another specific value for R₃ is

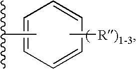

wherein R″ represents independently for each occurrence hydroxyl, halo, alkoxy, halo-alkoxy, C₁₋₃ alkyl-S(O)₂—NH—, —CO₂H, C₁₋₃ alkyl-C(O)—NH—, aryl or halo-substituted aryl, or hetereoaryl; or wherein there are two R″ attached to adjacent carbons, the two R″ groups taken together form

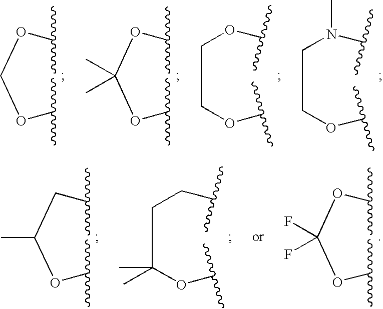

Other specific values for R₃ are

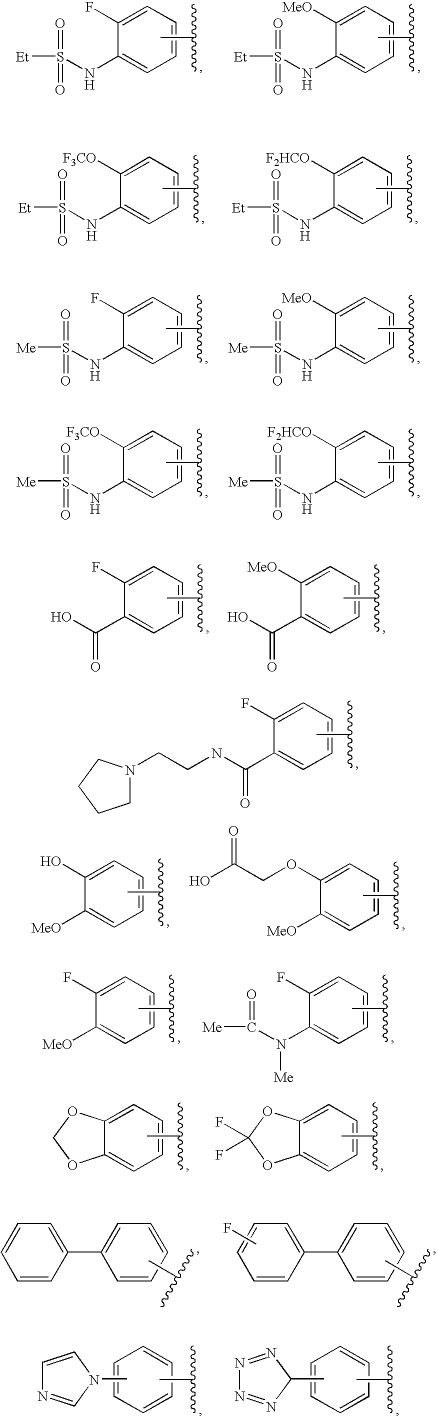

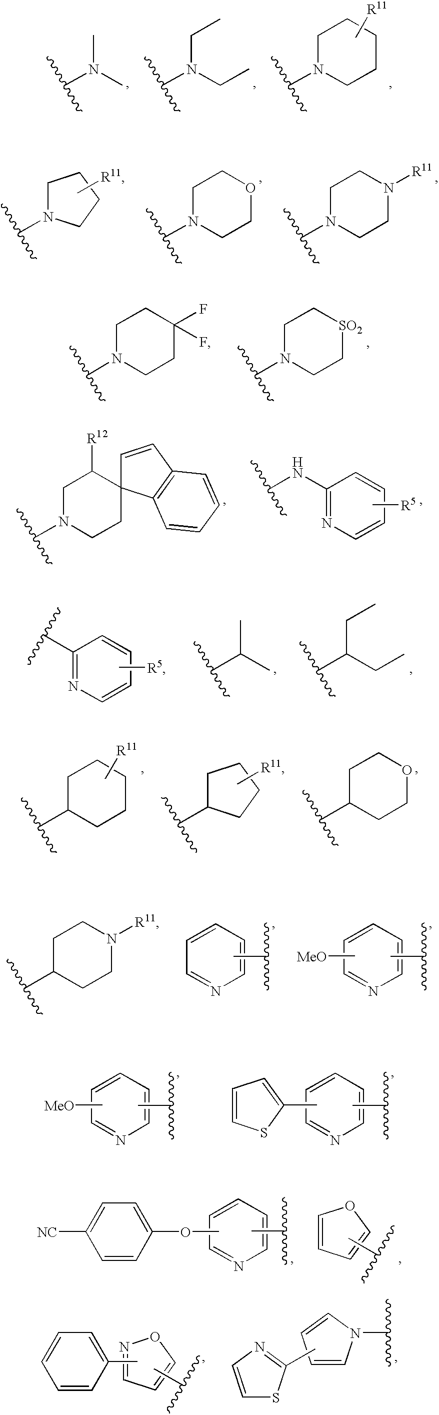

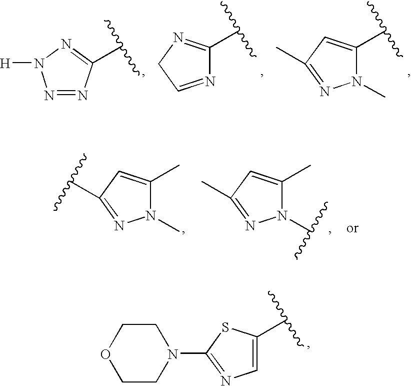

wherein R¹¹ is hydrogen, lower alkyl, hydroxyl, amino, alkoxy, or SO₂-lower alkyl; or an unsubstituted alicyclic, aryl, heterocyclic or heteroaryl; and R¹² is hydrogen or C₁₋₃ alkyl.

A specific group of compounds of formula I-A are compounds wherein:

R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅;

X is NR₁₀,

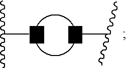

which is a 4, 5, or 6 membered ring wherein Z is N, CH, or C(C₁₋₃ alkyl); or NR₁₀(C₁-C₆alkylene) wherein (C₁-C₆alkylene) is optionally substituted with 1, 2, or 3 halo, methyl, or ethyl groups, or (C₁-C₆alkylene) is geminally substituted to form a cyclopropyl ring;

R₂ is aryl or heteroaryl, each of which is optionally substituted with 1, 2, or 3 occurrences of R₅;

Y is a direct bond or is CO, C₁₋₃ alkylene, C₃₋₆ cycloalkylene, arylene, heterocycloalkylene, or heteroarylene; each of which is optionally substituted with 1, 2, or 3 occurrences of R₅;

R₃ is hydrogen; an alkyl, alkenyl, or alkynyl group; or an cycloalkyl, aryl, heterocycloalkyl or heteroaryl; each of which, independently, is optionally substituted with 1, 2, or 3 occurrences of R₅;

R₄ is hydrogen; C₁₋₈ alkyl, alkenyl, or alkynyl optionally interrupted by oxygen or sulfur; cycloalkyl; alkoxy, arylalkoxy, or heteroarylalkoxy;

R₅, when present, may be halo, hydroxy, lower alkyl, C₁₋₃ alkoxy, —CO₂H, —CO₂C₁₋₃alkyl, cyano, aryl, heteroaryl, oxo, CF₃, O—CF₃, or O—CHF₂;

n is 0, 1 or 2; and

R₁₀ is hydrogen, or C₁₋₂ alkyl.

Another specific group of compounds of formula I-A are compounds wherein: R₁ is optionally substituted (C₁-C₃alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅.

X is NR₁₀,

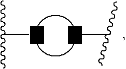

which is a 4, 5, or 6 membered ring wherein Z is N, CH, C(C₁₋₃ alkyl); or NR₁₀(C₁-C₆alkylene) wherein (C₁-C₆alkylene) is optionally substituted with 1, 2, or 3 halo, methyl, or ethyl groups, or is geminally substituted to form a cyclopropyl ring;

R₂ is aryl or heteroaryl, each of which is optionally substituted with 1 or 2 occurrences of R₅;

Y is a direct bond or is C₁₋₃ alkylene, C₃₋₆ cycloalkylene, arylene, heterocycloalkylene, or heteroarylene; each of which is optionally substituted with 1, 2, or 3 occurrences of R₅;

R₃ is cycloalkyl, aryl, heterocycloalkyl or heteroaryl; each of which is, independently, optionally substituted with 1, 2, or 3 occurrences of R₅;

R₄ is hydrogen;

R₅, when present, may be halo, hydroxy, lower alkyl, C₁₋₃ alkoxy, —CO₂H, —CO₂C₁₋₃alkyl, cyano, aryl, heteroaryl, oxo, CF₃, O—CF₃, or O—CHF₂;

n is 0, 1 or 2; and

R₁₀ is hydrogen or C₁₋₂ alkyl.

Another specific group of compounds of formula I-A are compounds wherein:

R₁ is optionally substituted (C₁-C₃alkylene)-R₁ₐ, wherein R₁ₐ is C₃₋₆ cycloalkyl, or C₃₋₆ heterocycloalkyl, each of which is optionally substituted with 1, 2, or 3 occurrences of R₅;

X is NR₁₀, NR₁₀(C₁-C₆alkylene), or

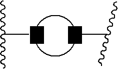

which is a 4, 5, or 6 membered ring wherein Z is N, CH, or C(C₁₋₃ alkyl);

R₂ is aryl or heteroaryl of which is optionally substituted with 1 or 2 occurrences of R₅;

Y is a direct bond, C₁₋₃ alkylene, C₃₋₆ cycloalkylene, arylene, heterocycloalkylene, or heteroarylene; each of which is optionally substituted with 1, 2, or 3 occurrences of R₅;

R₃ is cycloalkyl, aryl, heterocycloalkyl or heteroaryl; each of which, independently, when substituted, is optionally substituted with 1, 2, or 3 occurrences of R₅;

R₄ is hydrogen;

R₅, when present, may be halo, hydroxy, alkyl, C₁₋₃ alkoxy, cyano, aryl, heteroaryl, oxo, CF₃, O—CF₃, or O—CHF₂;

n is 0, 1 or 2; and

R₁₀ is hydrogen or C₁₋₂ alkyl.

Another specific group of compounds of formula I-A are compounds wherein:

R₁ is optionally substituted (C₁-C₃alkylene)-R₁ₐ, wherein R₁ₐ is C₃₋₆ cycloalkyl, or C₃₋₆ heterocycloalkyl, each of which is optionally substituted with 1, 2, or 3 occurrences of R₅;

X is NR₁₀,

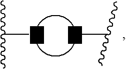

which is a 4, 5, or 6 membered ring wherein Z is N, CH, C(C₁₋₃ alkyl); or NR₁₀(C₁-C₆alkylene) wherein (C₁-C₆alkylene);

R₂ is aryl or heteroaryl each of which is optionally substituted with 1 or 2 occurrences of R₅;

Y is a direct bond or C₁₋₃ alkylene, which is optionally substituted with 1, 2, or 3 occurrences of R₅;

R₃ is cycloalkyl, aryl, heterocycloalkyl or heteroaryl; each of which, independently, is optionally substituted with 1, 2, or 3 occurrences of R₅;

R₄ is hydrogen;

R₅, when present, may be halo, alkyl, C₁₋₃ alkoxy, cyano, aryl, heteroaryl, oxo, CF₃, O—CF₃, or O—CHF₂;

n is 0, 1 or 2; and

R₁₀ is hydrogen or C₁₋₂ alkyl.

In an embodiment, the invention provides a compound of formula II

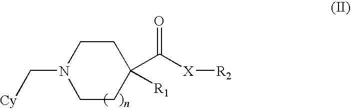

or a pharmaceutically acceptable salt thereof, wherein:

R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, or heteroaryl ring, each of which is optionally substituted with 1, 2, or 3 occurrences of R₅; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅;

X is a direct bond, NR₁₀, NR₁₀CO, NR₁₀SO₂,

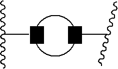

which is a 4, 5, or 6 membered ring wherein Z is N, CH, or C(C₁₋₃ alkyl); or NR₁₀(C₁-C₆alkylene), NR₁₀(C₁-C₆alkylene)SO₂—, wherein (C₁-C₆alkylene) is optionally substituted with 1, 2, or 3 halo, methyl, or ethyl groups, or is geminally substituted to form a cyclopropyl ring;

R₂ is cycloalkyl, aryl, heterocycloalkyl, heteroaryl, aralkyl, heteroaralkyl; each of which is optionally substituted with 1, 2, or 3 occurrences of R₅;

R₅, when present, is hydrogen, halogen, hydroxy, lower alkyl, lower alkenyl, cycloalkyl, C₁₋₃ alkoxy, —CO₂H, —CO₂C₁₋₃alkyl, cyano, aryl, heteroaryl, oxo, CF₃, O—CF₃, or O—CHF₂;

n is 0, 1 or 2;

R₁₀ is hydrogen, C₁₋₂ alkyl, or C₁-C₂alkenyl; and

Cy is an unsubstituted cyclic or bicyclic ring optionally having partial aromaticity and optionally having one or more heteroatoms.

In an embodiment, the invention provides a compound of formula III

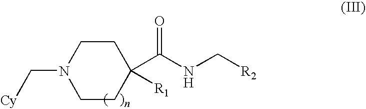

or a pharmaceutically acceptable salt thereof, wherein:

R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, or heteroaryl ring, each of which is optionally substituted with 1, 2, or 3 occurrences of R₅; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅;

R₂ is cycloalkyl, aryl, heterocycloalkyl, heteroaryl, aralkyl, or heteroaralkyl; each of which is optionally substituted with 1, 2, or 3 occurrences of R₅;

R₅, when present, is hydrogen, halogen, hydroxy, lower alkyl, lower alkenyl, cycloalkyl, C₁₋₃ alkoxy, —CO₂H, —CO₂C₁₋₃alkyl, cyano, aryl, heteroaryl, oxo, CF₃, O—CF₃, or O—CHF₂;

n is 0, 1 or 2;

R₁₀ is hydrogen, C₁₋₂ alkyl, or C₁₋₂ alkenyl; and

Cy is an unsubstituted cyclic or bicyclic ring optionally having partial aromaticity and optionally having one or more heteroatoms.

In an embodiment, the invention provides a compound of Formula IV-A:

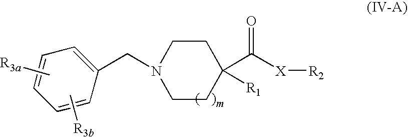

wherein:
m is 0 or 1;
R₃ₐ and R_{3b} are each independently hydrogen, halo, hydroxy, lower alkyl, lower alkenyl, cycloalkyl, C₁₋₃ alkoxy, cyano, or CF₃, or R₃ₐ and R_{3b} taken together form

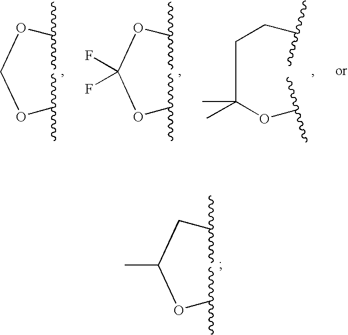

R₁, R₂, and X are as defined above.

In an embodiment, the invention provides a compound of Formula IV-B:

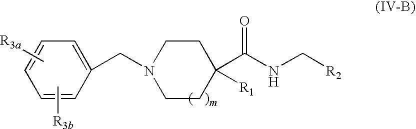

wherein the variables are as defined above.

In an embodiment, the invention provides a compound of Formula IV-C.

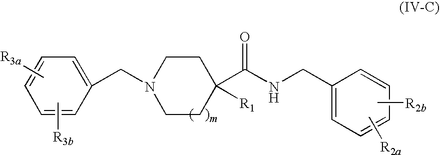

wherein R₂ₐ and R_{2b} are each independently hydrogen, halo, hydroxy, alkyl, haloalkyl, alkoxy, haloalkoxy, cyano, or CF₃; and

R₁, m, R₃ₐ, and R_{3b} are as defined above.

In an embodiment, the invention provides a compound of Formula IV-D.

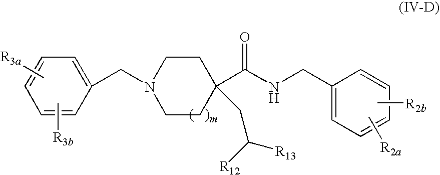

wherein R₁₂ and R₁₃ are each independently optionally substituted alkyl or taken together with the carbons to which they are attached form a 3, 4, 5, or 6-membered ring optionally containing one heteroatom selected from O, S, NH, or N-alkyl, which ring is optionally substituted with 1, 2, or 3 groups selected from halo, alkyl, alkoxy, or haloalkoxy; and

R₂ₐ, R_{2b}, R₃ₐ, R_{3b}, and m are as defined above.

In an embodiment, the invention provides a compound of Formula IV-E.

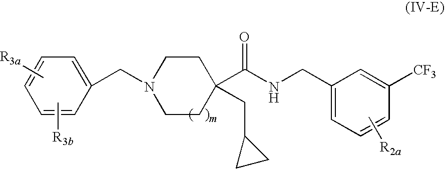

wherein R₂ₐ, R₃ₐ, R_{3b}, and m are as defined above.

The invention also provides pharmaceutical compositions comprising compounds selected from the group of formula I, and the use of these compounds and compositions in the prevention or treatment of diseases in which CCR2 chemokine receptors are involved.

The invention additionally provides a method for the treatment of inflammation, rheumatoid arthritis, lupus, systemic lupus erythematosus, atherosclerosis, restenosis, immune disorders, and transplant rejection in a mammal in need thereof, comprising administering to such mammal a therapeutically effective amount of a pharmaceutical composition containing a compound according to formula I in admixture with a pharmaceutically acceptable excipient, diluent, or carrier.

The invention further provides compositions comprising a compound of the invention and a pharmaceutically acceptable carrier.

The invention further provides methods of modulating the activity of a chemokine receptor comprising exposing said chemokine receptor with a compound of the invention.

The invention further provides methods of treating a disease associated with expression or activity of a chemokine receptor in a patient comprising administering to the patient a therapeutically effective amount of a compound of the invention.

The invention further provides a compound of Formula I for use in therapy.

The invention further provides use of a compound of Formula I for the manufacture of a medicament for the treatment of disease associated with expression or activity of a chemokine receptor.

The capacity of the compounds of the invention to antagonize CCR2 function can be determined using a suitable screen (e.g., high throughput assay). For example, an agent can be tested in an extracellular acidification assay, calcium flux assay, ligand binding assay or chemotaxis assay (see, for example, Hesselgesser et al., J Biol. Chem. 273(25):15687-15692 (1998), WO 00/05265 and WO 98/02151).

The compounds of Formula I of the invention, and compositions thereof are useful in the modulation of chemokine receptor activity, particularly CCR2. Accordingly, the compounds of the invention are those which inhibit at least one function or characteristic of a mammalian CCR2 protein, for example, a human CCR2 protein. The ability of a compound to inhibit such a function can be demonstrated in a binding assay (e.g., ligand binding or promoter binding), a signaling assay (e.g., activation of a mammalian G protein, induction of rapid and transient increase in the concentration of cytosolic free calcium), and/or cellular response function (e.g., stimulation of chemotaxis, exocytosis or inflammatory mediator release by leukocytes).

“Prodrug” includes compounds that are transformed in vivo to yield a compound of Formula (I) or a pharmaceutically acceptable salt, hydrate or solvate of the compound. The transformation may occur by various mechanisms, such as through hydrolysis in blood. For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of Formula (I) contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of Formula (I) incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino, morpholino, piperidin-1-yl or pyrrolidin-1-yl.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the invention. In addition, the invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

The compounds of Formula (I) may exist in unsolvated as well as solvated forms with pharmaceutically acceptable solvents such as water, ethanol, and the like, and it is intended that the invention embrace both solvated and unsolvated forms.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of Formula (I) can generally be prepared by following procedures analogous to those disclosed in the Schemes and/or in the Examples hereinbelow, by substituting an isotopically labeled reagent for a non-isotopically labeled reagent.

Compounds of the invention are useful MCP-1 antagonists; therefore, another embodiment of the invention is pharmaceutical compositions comprising a compound of the invention and a pharmaceutically acceptable excipient, diluent or carrier.

Another aspect of the invention is methods for treating or preventing diseases associated with monocyte and/or lymphocyte accumulation which comprises administering a therapeutically effective amount of a compound of the invention to an animal in need thereof CCR2 receptor antagonists have been shown to inhibit the binding of MCP-1 to its receptor. The compounds of the invention are therefore useful as agents for the treatment of inflammatory diseases, especially those associated with monocyte accumulation, including but not limited to, atherosclerosis, restenosis, gingivitis, glomerulonephritis, psoriasis, colitis, multiple sclerosis, pulmonary fibrosis, Crohn's disease, encephalomyelitis, sepsis, nephritis, asthma, rheumatoid arthritis, wound healing and tissue transplant rejection in animals (preferably humans). Accordingly, the compounds of the invention (including the pharmaceutical compositions and processes used therein) may be used in the manufacture of a medicament for the therapeutic applications described herein (e.g., treatment or prevention of diseases/conditions associated with monocyte and/or lymphocyte accumulation).

One or more additional pharmaceutical agents such as, for example, anti-viral agents, antibodies, anti inflammatory agents, immunosuppressants, chemotherapeutics can be used in combination with the compounds of the invention for treatment of chemokine receptor-associated diseases, disorders or conditions. These agents can be combined with the compounds of the invention in a single dosage form, or the agents can be administered simultaneously or sequentially as separate dosage forms.

Suitable antiviral agents contemplated for use in combination with the compounds of the invention can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine; zalcitabine; stavudine; lamivudine; abacavir; adefovir and lodenosine. Typical suitable NNRTIs include nevirapine; delaviradine; efavirenz; and (+)-calanolide A and B. Suitable protease inhibitors include; ritonavir; indinavir; nelfnavir; amprenavir; and lasinavir. Other antiviral agents include hydroxyurea, ribavirin, IL-2, IL-12, and pentafuside.

In some embodiments, anti-inflammatory or analgesic agents contemplated for use in combination with the compounds of the invention can comprise, for example, an opiate agonist, a lipoxygenase inhibitor such as an inhibitor of 5-lipoxygenase, a cyclooxygenase inhibitor such as a cyclooxygenase-2 inhibitor, an interleukin inhibitor such as an interleukin-1 inhibitor, an NNMA antagonist, an inhibitor of nitric oxide or an inhibitor of the synthesis of nitric oxide, a non-steroidal antiinflammatory agent, or a cytokine suppressing antiinflammatory agent, for example, such as acetaminophen, asprin, codiene, ibuprofen, indomethacin, morphine, naproxen, and the like. Similarly, the compounds of the invention may be administered with a pain reliever; a potentiator such as caffeine, an H2 antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedfine, or levo-desoxyephedrine; an antfitussive such as codeine, hydrocodone, caramiphen, carbetapentane, or dextramethorphan; a diuretic; and a sedating or non-sedating antihistamine.

“Individual”, “patient,” or “subject” are used interchangeably and include to any animal, including mammals, preferably mice, rats, other rodents, rabbits, dogs, cats, swine, cattle, sheep, horses, or primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the invention is desirably a mammal in whom modulation of chemokine receptor activity is desired. “Modulation” includes antagonism (e.g., inhibition), agonism, partial antagonism and/or partial agonism. In some embodiments, compounds of the invention are antagonists (e.g., inhibitors) of chemokine receptors.

In the present specification, the term “therapeutically effective amount” means the amount of the subject compound that will elicit the biological or medical response of a tissue, system, animal or human that is being sought by the researcher, veterinarian, medical doctor or other clinician. The compounds of the invention are administered in therapeutically effective amounts to treat a disease, e.g., as rheumatoid arthritis. A therapeutically effective amount of a compound is that amount which results in the inhibition of one or more of the processes mediated by the binding of a chemokine to a receptor such as CCR2 in a subject with a disease associated with aberrant leukocyte recruitment and/or activation. Typical examples of such processes include leukocyte migration, integrin activation, transient increases in the concentration of intracellular free calcium and granule release of proinflammatory mediators. Alternatively, a therapeutically effective amount of a compound is the quantity required to achieve a desired therapeutic and/or prophylactic effect, such as an amount which results in the prevention of or a decrease in the symptoms associated with a disease associated with aberrant leukocyte recruitment and/or activation.

Additional diseases or conditions of human or other species which can be treated with the inhibitors or modulators of chemokine receptor function of the invention, include, but are not limited to: inflammatory or allergic diseases and conditions, including respiratory allergic o diseases such as asthma, allergic rhinitis, hypersensitivity lung diseases, hypersensitivity pneumonitis, eosinophilic cellulitis (e.g., Well's syndrome), eosinophilic pneumonias (e.g., Loeffler's syndrome, chronic eosinophilic pneumonia), eosinophilic fasciitis (e.g., Shulman's syndrome), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), eosinophilia-myalgia syndrome due to the ingestion of contaminated tryptophan, insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such as an dermatitis, eczema, atopic dermatitis, allergic contact dermatitis, urticaria; vasculitis (e.g., necrotizing, cutaneous, and hypersensitivity vasculitis); eosinophilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, restenosis, certain hematologic malignancies, cytokine-induced toxicity (e.g., septic shock, endotoxic shock), polymyositis, dermatomyositis. Example viral infections include HIV infection.

Suitable pharmaceutical agents that may be used in combination with the compounds of the invention include nutraceuticals, cholesterol absorption inhibitors, HMG-CoA reductase inhibitors, MTP/Apo B secretion inhibitors, HMG-CoA synthase inhibitors, HMG-CoA reductase transcription inhibitors, HMG-CoA reductase translation inhibitors, CETP inhibitors, squalene synthetase inhibitors, squalene epoxidase inhibitors, squalene cyclase inhibitors, combined squalene epoxidase/squalene cyclase inhibitors, ACAT inhibitors, lipase inhibitors (including pancreatic lipase inhibitors and gastric lipase inhibitors) and peroxisome proliferator-activated receptor (PPAR) agonists (preferably PPARα agonists).

Any naturally occurring compound that acts to lower plasma cholesterol levels may be administered in combination with the compounds of the invention. These naturally occurring compounds are referred to herein as “nutraceuticals” and include, for example, garlic extract and niacin.

Any cholesterol absorption inhibitor may be used as the second compound in the combination aspect of this invention. The term “cholesterol absorption inhibition” refers to the ability of a compound to prevent cholesterol contained within the lumen of the intestine from entering into the intestinal cells and/or passing from within the intestinal cells into the blood stream. Such cholesterol absorption inhibition activity is readily determined by those skilled in the art according to standard assays (see, e.g., J. Lipid Res. 34, 377-395 (1993)). Suitable cholesterol absorption inhibitors are well known to those skilled in the art and include compounds such as steroidal glycosides which are described in WO 94/00480.

Any HMG-CoA reductase inhibitor may be used as the second compound in the combination aspect of this invention. The term “HMG-CoA reductase inhibitor” refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (see, e.g., Meth. Enzymol., 71, 455-509 (1981) and references cited therein). Suitable HMG-CoA reductase inhibitors include statins, e.g., lovastatin; simvastatin; fluvastatin; pravastatin; rivastatin; atorvastatin and hemicalcium salts thereof; itavostatin (aka nisvastatin, pitavastatin, NK-104) and rosuvastatin.

Any MTP/Apo B secretion (microsomal triglyceride transfer protein and/or apolipoprotein B secretion) inhibitor may be used as the second compound in the combination aspect of this invention. The term “MTP/Apo B secretion inhibitor” refers to compounds which inhibit the secretion of triglycerides, cholesteryl ester, and phospholipids. Such inhibition is readily determined by those skilled in the art according to standard assays (e.g., Wetterau, J. R., Science, 258, 999 (1992)). A variety of these compounds are known to those skilled in the art. Suitable MTP/Apo B secretion inhibitors include biphenyl-2-carboxylic acid-tetrahydro-isoquinolin-6-yl amide derivatives, e.g., as described in U.S. Pat. Nos. 5,919,795 and 6,121,283.

Any HMG-CoA synthase inhibitor may be used as the second compound in the combination aspect of this invention. The term “HMG-CoA synthase inhibitor” refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (e.g., Meth Enzymol., 35, 155-160 (1975): Meth. Enzymol. 110, 19-26 (1985) and references cited therein). HMG-CoA synthase inhibitors known to those skilled in the art, e.g., as described in U.S. Pat. Nos. 5,120,729 (beta-lactam derivatives); 5,064,856 (spiro-lactone derivatives); and 4,847,271 (oxetane compounds such as 11-(3-hydroxymethyl-4-oxo-2-oxetayl)-3,5,7-trimethyl-2,4-undeca-dienoic acid derivatives.)

Any compound that decreases HMG-CoA reductase gene expression may be used as the second compound in the combination aspect of this invention. These agents may be HMG-CoA reductase transcription inhibitors that block or decrease the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such regulation is readily determined by those skilled in the art according to standard assays (see, e.g., Meth. Enzymol., 110, 9-19 (1985)). Inhibitors of HMG-CoA reductase gene expression are well known to those skilled in the art, e.g., U.S. Pat. No. 5,041,432 (15-substituted lanosterol derivatives); and oxygenated sterols that suppress synthesis of HMG-CoA reductase (Prog. Lip. Res., 32, 357-416 (1993)).

Any compound having activity as a CETP inhibitor can serve as the second compound in the combination therapy aspect of the invention. The term “CETP inhibitor” refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art; e.g., U.S. Pat. Nos. 6,140,343 (4-amino substituted-2-substituted-1,2,3,4-tetrahydroquinolines); 5,512,548 (polypeptide derivatives) and CETP-inhibitory rosenonolactone derivatives and phosphate-containing analogs of cholesteryl ester (J. Antibiot., 49(8), 815-816 (1996), and Bioorg. Med. Chem. Lett., 6, 1951-1954 (1996), respectively.)

Any squalene synthetase inhibitor may be used as the second compound of this invention. The term “squalene synthetase inhibitor” refers to compounds which inhibit the condensation of 2 molecules of farnesylpyrophosphate to form squalene, catalyzed by the enzyme squalene synthetase. Inhibition is readily determined by those skilled in the art according to standard assays (e.g., Meth. Enzymol, 15, 393-454 (1969) and Meth. Enzymol, 110, 359-373 (1985)). A variety of these compounds are known to those skilled in the art, e.g., in U.S. Pat. No. 5,026,554, disclosing fermentation products of the microorganism MF5465 (ATCC 74011) including zaragozic acid. A summary of other squalene synthetase inhibitors has been compiled (Curr. Op. Ther. Patents, 3, 861-4 (1993)).

Any squalene epoxidase inhibitor may be used as the second compound in the combination aspect of this invention. “Squalene epoxidase inhibitor” refers to compounds which inhibit the bioconversion of squalene and molecular oxygen into squalene-2,3-epoxide, catalyzed by the enzyme squalene epoxidase. Such inhibition is readily determined by those skilled in the art according to standard assays (e.g., Biochim Biophys Acta, 794, 466-471 (1984)). A variety of these compounds are well known to those skilled in the art, e.g., U.S. Pat. Nos. 5,011,859 and 5,064,864 (fluoro analogs of squalene); EP publication 395,768 A (substituted allylamine derivatives); PCT publication WO 9312069 (amino alcohol derivatives); and U.S. Pat. No. 5,051,534 (cyclopropyloxy-squalene derivatives.)

Any squalene cyclase inhibitor may be used as the second component in the combination aspect of this invention. The term “squalene cyclase inhibitor” refers to compounds which inhibit the bioconversion of squalene-2,3-epoxide to lanosterol, catalyzed by the enzyme squalene cyclase. Inhibition is readily determined by those skilled in the art according to standard assays (e.g., FEBS Lett., 244, 347-350 (1989)). Squalene cyclase inhibitors are well known to those skilled in the art, e.g., U.S. Pat. No. 5,580,881 (1,2,3,5,6,7,8,8a-octahydro-5,5,8a-trimethyl-(8aβ)-6-isoquinolineamine derivatives.)

Any combined squalene epoxidase/squalene cyclase inhibitor may be used as the second component in the combination aspect of this invention. The term “combined squalene epoxidase/squalene cyclase inhibitor” refers to compounds that inhibit the bioconversion of squalene to lanosterol via a squalene-2,3-epoxide intermediate. In some assays it is not possible to distinguish between squalene epoxidase inhibitors and squalene cyclase inhibitors. However, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of squalene epoxidase/squalene cyclase inhibitors are well known to those skilled in the art, e.g., U.S. Pat. Nos. 5,084,461 and 5,278,171 (azadecalin derivatives); EP publication 468,434 (piperidyl ether and thio-ether derivatives such as 2-(1-piperidyl)pentyl isopentyl sulfoxide and 2-(1-piperidyl)ethyl ethyl sulfide); PCT publication WO 94/01404 (acyl-piperidines such as 1-(1-oxopentyl-5-phenylthio)-4-(2-hydroxy-1-methyl)-ethyl)piperidine; and U.S. Pat. No. 5,102,915 (cyclopropyloxy-squalene derivatives.)

Any ACAT inhibitor can serve as the second compound in the combination therapy aspect of this invention. The term “ACAT inhibitor” refers to compounds that inhibit the intracellular esterification of dietary cholesterol by the enzyme acyl CoA: cholesterol acyltransferase. Such inhibition may be determined readily by one of skill in the art according to standard assays, such as the method described in Heider et al., Journal of Lipid Research., 24,1127 (1983). A variety of these compounds are well known to those skilled in the art, e.g., U.S. Pat. No. 5,510,379 (carboxysulfonates), WO 96/26948 and WO 96/10559 (urea derivatives having ACAT inhibitory activity); DL-melinamide (GB Pat. No. 1,123,004 and Japan. J. Pharmacol., 42, 517-523 (1986); 2,2-dimethyl-N-(2,4,6-trimethoxyphenyl)dodecanamide (U.S. Pat. No. 4,716,175); and N-[2,6-bis(1-methylethyl)phenyl]-N′-[[1-(4-dimethylaminophenyl)cyclopentyl]-methyl]urea (U.S. Pat. No. 5,015,644.)

Any lipase inhibitor may be used in combination with the compounds of the invention. The term “lipase inhibitor” refers to a compound that inhibits the metabolic cleavage of dietary triglycerides into free fatty acids and monoglycerides. Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a monoglyceride and a fatty acid. The resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays.

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol, 286, 190-231 (1997)).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. Gastric lipase inhibition activity is readily determined by those skilled in the art according to standard assays (e.g., Methods Enzymol, 286, 190-231 (1997)).

A variety of gastric and/or pancreatic lipase inhibitors are well known to one of ordinary skill in the art, e.g., lipstatin, tetrahydrolipstatin (orlistat), valilactone, esterastin, ebelactone A and ebelactone B; N-3-trifluoromethylphenyl-N′-3-chloro-4′-trifluoromethylphenylurea and derivatives thereof (U.S. Pat. No. 4,405,644); esteracin; cyclo-O,O′-[(1,6-hexanediyl)-bis-(iminocarbonyl)]dioxime; and bis(iminocarbonyl)dioximes.

(2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof (U.S. Pat. No. 4,598,089) tetrahydrolipstatin (U.S. Pat. Nos. 5,274,143; 5,420,305; 5,540,917; and 5,643,874); FL-386, 1-[4-(2-methylpropyl)cyclohexyl]-2-[(phenylsulfonyl)oxy]-ethanone and substituted sulfonate derivatives related thereto (U.S. Pat. No. 4,452,813); and WAY-121898, 4-phenoxyphenyl-4-methylpiperidin-1-yl-carboxylate, and carbamate esters and pharmaceutically acceptable salts thereof (U.S. Pat. Nos. 5,512,565; 5,391,571 and 5,602,151); valilactone (Kitahara, et al.)

Other compounds that are marketed for hyperlipidemia may also be used in combination with compounds of the invention, including those compounds marketed for hypercholesterolemia which are intended to help prevent or treat atherosclerosis, for example, bile acid sequestrants, such as Welchol®, Colestid®, LoCholest® and Questran®; and fibric acid derivatives, such as Atromid®, Lopid® and Tricor®. Examples of bile acid sequestrants are also discussed in U.S. Pat. Nos. 3,692,895 and 3,803,237 (colestipol); U.S. Pat. No. 3,383,281 (cholestyramine) and Casdorph R. in Lipid Pharmacology, 1976; 2:222-256, Paoletti C., Glueck J., eds. Academic Press, N.Y.

Any peroxisome proliferator-activated receptor (PPAR) agonists (preferably PPARα agonists) can be used in combination with compounds of the invention. Suitable PPAR agonists include fibrates (e.g., bezafibrate, ciprofibrate, clofibrate, fenofibrate, and gemfibrozil, which are all commercially available) and glitazones (e.g., pioglitazone, and rosiglitazone, which are both commercially available). Gemfibrozil is described in U.S. Pat. No. 3,674,836; bezafibrate is described in U.S. Pat. No. 3,781,328; clofibrate is described in U.S. Pat. No. 3,262,850; and fenofibrate is described in U.S. Pat. No. 4,058,552.

Other compounds that may be used in combination with the compounds of the invention include NSAIDs, COX-2 inhibitors, and antiallergics. Suitable nonsteroidal anti-inflammatory drugs (NSAIDS) include compounds such as ibuprofen (Motrin™, Advil™), naproxen (Naprosyn™), sulindac (Clinori™), diclofenac (Voltare™), piroxicam (Feldene™), ketoprofen (Orudis™), diflunisal (Dolobid™), nabumetone (Relafen™), etodolac (Lodine™), oxaprozin (Daypr™), and indomethacin (Indocin™). Suitable COX-2 inhibitors (cyclooxygenase enzyme inhibitors) include compounds such as celecoxib (Celebrex™) and rofecoxib (Vioxx™)

“Combination therapy” (or “co-therapy”) includes the administration of a 5-HT modulator of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The compounds of the invention and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

The compounds of the invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

The compounds of the invention can be administered to a patient at dosage levels in the range of from about 0.01 to about 100 mg per day. As used herein, the term “unit dose” or “unit dosage” refers to physically discrete units that contain a predetermined quantity of a compound of the invention calculated to produce a desired therapeutic effect. The dosage to be administered may vary depending upon the physical characteristics of the patient, the severity of the patient's symptoms, and the means used to administer the drug. The specific dose for a given patient is usually set by the judgment of the attending physician. It is also noted that the compounds of the invention can be used in sustained release, controlled release, and delayed release formulations, which forms are also well known to one of ordinary skill in the art.

The compositions and combination therapies of the invention may be administered in combination with a variety of pharmaceutical excipients, including stabilizing agents, carriers and/or encapsulation formulations as described herein.

Aqueous compositions of the present invention comprise an effective amount of the peptides of the invention, dissolved or dispersed in a pharmaceutically acceptable carrier or aqueous medium.

“Pharmaceutically or pharmacologically acceptable” include molecular entities and compositions that do not produce an adverse, allergic or other untoward reaction when administered to an animal, or a human, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards.

The pharmaceutical compositions of this invention may be used in the form of a pharmaceutical preparation, for example, in solid, semisolid or liquid form, which contains one or more of the compound of the invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the invention may be incorporated for administration orally or by injection include aqueous solution, suitably flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

For treating clinical conditions and diseases noted above, the compound of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The preparation of an aqueous composition that contains a composition of the invention or an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Pharmaceutically acceptable salts include acid addition salts and which are formed with inorganic acids such as, for example, hydrochloric, hydrobromic, boric, phosphoric, sulfuric acids or phosphoric acids, or such organic acids as acetic, oxalic, tartaric, maleic, fumaric, citric, succinic, mesylic, mandelic, succinic, benzoic, ascorbic, methanesulphonic, a-keto glutaric, a-glycerophosphoric, glucose-1-phosphoric acids and the like. Salts formed with the free carboxyl groups can also be derived from inorganic bases such as, for example, sodium, potassium, ammonium, calcium, magnesium, or ferric hydroxides, and such organic bases as isopropylamine, trimethylamine, histidine, procaine and the like. Other examples of pharmaceutically acceptable salts include quaternary derivatives of the compounds of Formulae I, II, III or IV such as the compounds quaternized by compounds Rₓ-T wherein Rₓ is C₁₋₆ alkyl, phenyl-C₁₋₆ alkyl or C₅₋₇ cycloalkyl, and T is a radical corresponding to an anion of an acid. Suitable examples of Rₓ include methyl, ethyl and n- and iso-propyl; and benzyl and phenethyl. Suitable examples of T include halide, e.g., chloride, bromide or iodide. Yet other examples of pharmaceutically acceptable salts also include internal salts such as N-oxides.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including cremes, lotions, mouthwashes, inhalants and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal formulations; time-release capsules; and any other form currently used, including creams.

The use of sterile formulations, such as saline-based washes, by surgeons, physicians or health care workers to cleanse a particular area in the operating field may also be particularly useful. Therapeutic formulations in accordance with the present invention may also be reconstituted in the form of mouthwashes, or in conjunction with antifungal reagents. Inhalant forms are also envisioned. The therapeutic formulations of the invention may also be prepared in forms suitable for topical administration, such as in cremes and lotions.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals. For example, for parenteral administration, a suitably buffered, and if necessary, isotonic aqueous solution would be prepared and used for intravenous, intramuscular, subcutaneous or even intraperitoneal administration. One dosage could be dissolved in 1 ml of isotonic NaCl solution and either added to 1000 ml of hypodermolysis fluid or injected at the proposed site of infusion, (see for example, Remington's Pharmaceutical Sciences 15th Edition, pages 1035-1038 and 1570-1580).

In certain embodiments, active compounds may be administered orally. This is contemplated for agents which are generally resistant, or have been rendered resistant, to proteolysis by digestive enzymes. Such compounds are contemplated to include chemically designed or modified agents; dextrorotatory peptides; and peptide and liposomal formulations in time release capsules to avoid peptidase and lipase degradation.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Advantageously, the invention also provides kits for use by a consumer having, or at risk of having, a disease or condition associated with monocyte, lymphocyte or leukocyte accumulation, which can be ameliorated by a CCR2 antagonist. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units.

Since the invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the invention and a second pharmaceutical agent as described above. The kit comprises a container (e.g., a divided bottle or a divided foil packet). Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Activity of Invention Compounds

The suitability of compounds of the invention for the uses described herein may be determined by methods and assays known in the art. The following tests are found particularly advantageous.

For determining the ability of compounds to effect chemotaxis, assays in two formats may be used:

Methods using Boyden chambers: Cells are washed twice in RPMI with 0.1% BSA and starved for 2 hours in RPMI 0.1% BSA at 37° C. in 5% CO₂. After starving, the cells are resuspended at 1×10⁶ cell/ml (in some cases, the cell density may be varied in order to investigate the optimal cell numbers that can be used in the assay) in RPMI 0.1% BSA. About 1×10⁵/100 μl cells are added into the upper wells of the Boyden chamber apparatus with 8 μm pore size filter. Chemotactic factors are diluted to the indicated concentrations in RPMI 0.1% BSA, and 200 μl of the mixture is added into the lower wells of the Boyden chambers. After 2 hours at 37° C. in 5% CO₂, the cells remaining in the upper chamber are removed. Migrated cells in the lower surface of the filters are fixed with Methanol and stained with 15% Giemsa. The cells are counted in 10 high power fields.

Methods using neuroprobes: Cells are washed twice in RPMI with 0.1% BSA and starved for 2 hours in RPMI 0.1% BSA at 37° C. in 5% CO₂. After starving, the cells are resuspended at 1×10⁶ cell/ml in RPMI 0.1% BSA and stained with 1 μg/ml Calcein AM for 30 min at 37° C. in 5% CO₂. Stained cells are washed twice with PBS and resuspended at 1×10⁶ cell/ml in RPMI 0.1% BSA. About 25 μl of the cells are added into the upper chambers of the 96-well neuroprobe plates with an 8 μm pore size filter. Chemotactic factors are diluted to the indicated concentrations in RPMI 0.1% BSA, and 30 μl of the mixture is added into the lower chambers of the 96-well neuroprobe plate. After 2 hours at 37° C. in 5% CO₂, the cells remaining in the upper chambers are removed and rinsed with PBS once. Migrated cells in the lower surface of the filters and low chamber are determined as the fluorescent value measured at λ450-530 by Cytofluor.

For determining the ability of compounds to bind to CCR2 and to block MCP-1 binding, the following assay is useful. To maximize reliability and reproducibility Human recombinant CHO-K1 cells that overexpress CCR2 are used in this assay. Increasing concentrations of antagonist is incubated with cells in the presence of 1% DMSO, 25 mM HEPES pH:7.4, 1 mM CaCl₂, 0.5% BSA, 5 mM MgCl₂, 0.1% sodium azide. The potency of the compounds are calculated as a function of decreasing quantity of ¹²⁵I-labeled MCP-1 (1 nM) ability to bind to the receptor. Reference standards are run as an integral part of each assay to ensure the validity of the results obtained. Where presented, IC₅₀ values are determined by a non-linear, least squares regression analysis using Data Analysis Toolbox (MDL Information Systems, San Leandro, Calif., USA). Where inhibition constants Kᵢ are presented, the Kᵢ values are calculated using the equation of Cheng and Prusoff (Cheng, Y., Prusoff, W. H., Biochem. Pharmacol. 22:3099-3108, 1973) using the observed IC₅₀ of the tested compound, the concentration of radioligand employed in the assay, and the historical values for the K_{D} of the ligand (obtained experimentally at MDS Pharma Services). Where presented, the Hill coefficient (n_{H}), defining the slope of the competitive binding curve, is calculated using Data Analysis Toolbox. Hill coefficients significantly different than 1.0 may suggest that the binding displacement does not follow the laws of mass action with a single binding site. Where IC₅₀, Kᵢ, and/or n_{H} data are presented without Standard Error of the Mean (SEM), data are insufficient to be quantitative, and the values presented (K₁, IC₅₀, n_{H}) should be interpreted accordingly.

The efficacy of compounds of the invention may further be determined using a (GTP γS) assay in which the potency of a given antagonist is assessed by the inhibition observed in the binding of radioactively labeled GTP to the cell membranes or whole cells. Compounds are tested at several concentrations in duplicate (n=2) to obtain a dose-response curve and estimated ICₛₒ values. The assay buffer is 20 mM HEPES pH 7.4; 100 mM NaCl, 10 μg/ml saponin, 1 mM MgCl₂. The assay is performed on membranes that are thawed on ice and diluted in assay buffer to give 250 μg/ml (5 μg/20 μl), keep on ice. 20 μl of 5 uM GDP (1 μM final). 10 μl of antagonist at increasing concentrations is added successively in the wells of an Optiplate (Perkin Elmer) together with 20 μl of membranes (5 μg) and preincubated for 15 min. at room temperature. To this 10 μl of assay buffer or of reference agonist (MCP-1 R&D Systems, 279-MC) at EC₈₀ (10×), 20 μl of GTPg³⁵S (0.1 nM final), 20 μl of PVT-WGA beads (Amersham, RPNQ001). Control antagonist RS 102895 (Tocris, 2089) diluted in assay buffer is used in each assay as a reference. The plate is covered with a topseal, placed on an orbital shaker for 2 min., incubated for 30 min. at room temperature, centrifuged for 10 min. at 2000 rpm, incubated for 2 h at room temperature and counted in a TopCount (Packard) for 1 min.

Methods for preparing the compounds of the invention are illustrated in the following synthetic schemes and example(s). The following schemes, examples and biological data are given for the purpose of illustrating the invention, but not for limiting the scope or spirit of the invention.

Preparation of Invention Compounds

The following examples are given for the purpose of illustrating the invention, but not for limiting the scope or spirit of the invention. Compounds of the invention may be prepared as described in the following schemes.

### General Schemes

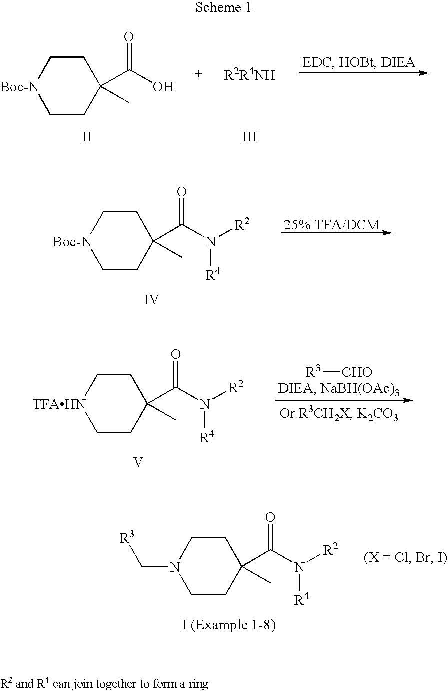

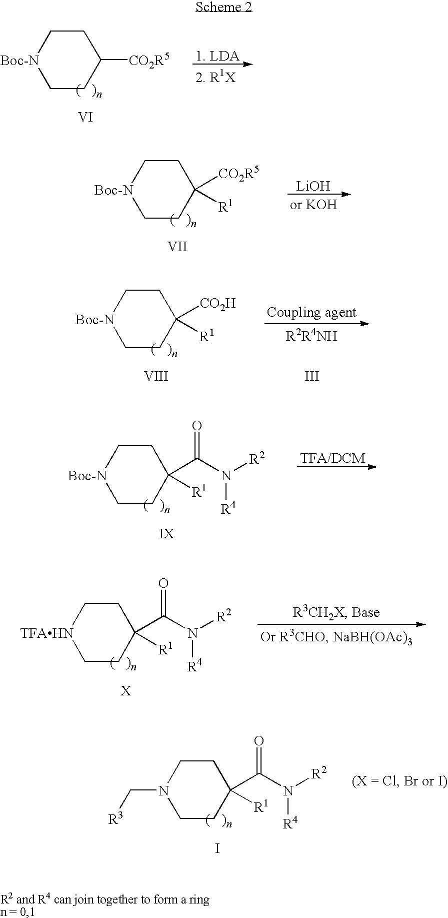

### EXPERIMENTAL

### A: General Procedure for Amide Formation

### Step 1 of Scheme 1

1-(tent-Butoxycarbonyl)-4-methylpiperidine-4-carboxylic acid (II) (1.0 mmol), HOBt (1.24 mmol) and EDC (1.34 mmol) were dissolved in anhydrous THF (15 mL), followed by addition of DIEA (4.0 mmol). Sonication of the mixture may be needed to assist solubility. After stirring for 5 to 10 min., a solution of amine (III) (1.1 mmol) in THF (2 mL) was added. The reaction mixture was stirred at room temperature for 3-5 h then diluted with 80 mL of ethyl acetate. The organic layer was washed sequentially with 5% aqueous acetic acid (30 mL×2), saturated aqueous sodium bicarbonate (30 mL×2), water (30 mL) and brine (30 mL). The organic layer was dried over anhydrous sodium sulfate, filtered and concentrated in vacuo to yield a crude IV. Purification by silica chromatography on an ISCO system gave the desired product IV with satisfactory purity.

### B: General Procedure for Removal of Boc Group

### Step 2 of Scheme 1

Boc-protected amine IV (1.0 mmol) was stirred in CH₂Cl₂ (12 mL) at 0° C. and trifluoroacetic acid (TFA) (3 mL) was added slowly. The ice bath was immediately removed after the addition of TFA. The resulting solution was stirred at room temperature for 1.5 h then a small amount of isopropyl alcohol (1 mL) was added. Concentration of the solution under reduced pressure gave the unprotected amine V as the trifluororacetic acid salt, which was used without further purification in the next step. Compound V can be converted to a free base through a standard basic work-up (NaHCO₃).

### C: General Procedure for Reductive Amination

### Step 3 of Scheme 1

A mixture of aldehyde (1.0 mmol), acetic acid (1.5 mmol) and amine V (1.2-1.5 mmol) in DCM/MeOH (1:2, 12 mL) was stirred at room temperature for 1 h. Sodium triacetoxyborohydride (2-3 mmol) was added and the reaction mixture was stirred for 16 h at room temperature. After concentration of solvent under reduced pressure, the resulting residue was dissolved in ethyl acetate, then washed with water and brine. The organic extract was dried, filtered and concentrated. The crude product was purified either by silica chromatography on an ISCO system (eluted at 4-8% methanol in DCM) or by reverse phase preparative HPLC (Phenomenex reverse phase Luna 5u C18(2) column, 60×21.2 mm ID, mobile phase: A=0.05% TFA in water; B=0.05% TFA in acetonitrile). The flow rate was 10-12 mL/min) to yield the desired final product with purity greater than 95%.

### D: General Procedure of Alkylation of Ester

### Step 1 of Scheme 2

To a solution of Boc-protected amine VI (4.0 mmol) cooled at −78° C. in THF was added a solution of freshly prepared LDA (4.0 mmol). The solution was allowed to stir at −78° C. for 15 min and at 0° C. for 45 min. The appropriate alkyl halide was added and stirring was continued overnight. The crude product was purified by silica chromatography on an ISCO system (5-10% EtOAc/hexanes) to collect compounds of structure VII in 50-75% yield.

### E: General Procedure of Saponification

### Step 2 of Scheme 2

Ester VII (2.5 mmol) was heated with LiOH (25 mmol) in a mixture of MeOH/H₂O/THF (2.5/2.5/1.0) at 90° C. for 2-16 h. The solvent was removed under vacuum. The residue was washed with ethyl acetate to remove unreacted ester. The separated aqueous layer was acidified to pH 4 with 1M HCl, and then extracted with ethyl acetate (×3). The acid of formula VIII was collected after solvent removal and/or purified by silica chromatography in 5% MeOH/DCM.

### F: General Procedure of Alkylation of Piperidine and Pyrrolidine

### Step 5 of Scheme 2

A mixture of piperidine or pyrrolidine intermediate X (1.0 mmol), alkyl/aryl halide (1.2 mmol), diisopropylethylamine (1.5 mmol) and potassium carbonate (2.5 mmol) in DMF (5-6 mL) was irradiated in a Microwave instrument at 110° C. for 20-30 min (Personal Chemistry Emrys™ Optimizer microwave reactor). The reaction mixture was cooled and diluted with ethyl acetate. The combined organic layers were washed with brine (×3), then dried over Na₂SO₄, filtered and concentrated under reduced pressure. The crude product was purified either by column chromatography on ISCO system (the final product as free base) or by reverse phase preparative HPLC (Phenomenex reverse phase Luna 5 u C18(2) column, 60×21.2 mm ID, mobile phase: A=0.05% TFA in water; B=0.05% TFA in acetonitrile. The flow rate was 10-12 mL/min) to yield the desired final product as trifluoroacetic salt with purity greater than 95%.

### G: General Procedure for N—C Bond Coupling Reaction

Intermediate (II) (1.0 mmol), Aryl halide (1.2 mmol), Pd(dppf)Cl₂ (0.03 mmol), dppf (0.045 mmol) and sodium tert-butoxide (1.5 mmol) was mixed in toluene (2 mL). The mixture was purged with N₂ gas for 3-5 min and heated at 120° C. for 30 min under microwave irradiation (Personal Chemistry Emrys™ Optimizer microwave reactor). Upon completion of the reaction, the reaction mixture was concentrated under reduced pressure. The residues was diluted with ethyl acetate, and then washed with NH₄Cl, water and brine. The crude product was purified either by column chromatography on ISCO system (the final product as free base) or by reverse phase preparative HPLC to yield the desired final product as trifluoroacetic salt with purity greater than 95%.

### Intermediate IV

### t-Butyl 4-(3,5-bis(trifluoromethyl)benzylcarbamoyl)-4-methylpiperidine-1-carboxylate

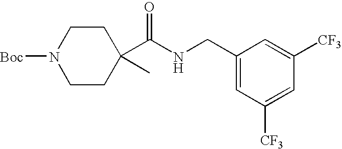

The title compound was prepared according to general procedure A described above in connection with Scheme 1. 1-(t-Butoxycarbonyl)-4-methylpiperidine-4-carboxylic acid (1.13 g, 4.6 mmol), (3,5-bis(trifluoromethyl)phenyl)methanamine (1.24 g, 5.10 mmol), EDCI (1.16 g, 6.03 mmol), HOBt (0.75 g, 5.57 mmol) and DIEA (2.39 g, 18.6 mmol) were stirred in DCM for 16 h at RT. The reaction was washed with water (10 mL). The solvent was removed under vacuum and the crude purified by silica chromatography in 10% EtOAc in hexanes to obtain the desired product (1.7 g, 76% yield): ¹H NMR (400 MHz, CDCl₃): δ 7.77 (s, 1H), 7.70 (s, 2H), 6.73 (t, 1H), 4.55 (d, 2H) 3.56 (m, 2H), 3.24 (t, 2H), 2.03 (m, 2H), 1.47 (m, 2H), 1.43 (s, 9H), 1.24 (s, 3H); MS (ESI) m/z: Calculated for C₂₁H₂₆F₆N₂O₃: 468.2. found: 412.9 (M+t-butyl)⁺.

### Intermediate V

### N-(3,5-Bis(trifluoromethyl)benzyl)-4-methylpiperidine-4-carboxamide 2,2,2-trifluoroacetate

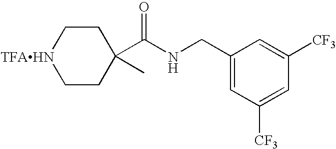

The title compound was prepared according to general procedure B described above in connection with Scheme 1. t-Butyl 4-(3,5-bis(trifluoromethyl)benzylcarbamoyl)-4-methylpiperidine-1-carboxylate (0.89 g, 1.91 mmol) was dissolved and stirred in 3 mL of 25% TFA in DCM solution for 2 h at 0° C. The solvent was removed under vacuum to obtain the desired product (0.9 g, 98% yield): ¹H NMR (400 MHz, CDCl₃): δ; 8.32 (bs, 2H, NH₂), 7.81 (s, 1H), 7.70 (s, 2H), 6.70 (t, 1H), 4.58 (d, 2H), 3.33 (m, 2H), 3.27 (m, 2H), 2.27 (d, 2H), 1.83 (t, 2H), 1.35 (s, 3H); MS (ESI) m/z: Calculated for C₁₆H₁₈F₆N₂O: 368.1. found: 369.1 (M+H)⁺.

### Intermediate VIIa

### 1-t-Butyl 4-methyl 4-(cyclopropylmethyl)piperidine-1,4-dicarboxylate

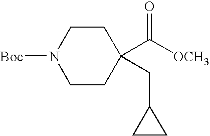

The title compound was prepared according to general procedure D described above in connection with Scheme 2. 1-t-Butyl 4-methyl piperidine-1,4-dicarboxylate (10.0 g, 41.1 mmol) was dissolved in 80 mL THF under argon. 2M LDA in heptane/THF/Ethylbenzene (22.6 mL, 45.2 mmol) was added in 30 minutes between −78° C. and −68° C. The reaction mixture was stirred at −78° C. for 45 min. Cyclopropylmethylbromide (5.9 mL, 61.7 mmol) neat was added over 11 minutes at −78° C. The reaction was slowly warmed to RT over 20 h. The reaction mixture was cooled to −20° C. and quenched with 50 mL 10% NH₄Cl. The aqueous layer was extracted with 50 mL EtOAc. The organic layer was washed with 50 mL brine and then dried over Na₂SO₄. The solvent was evaporated and the resulting residue was purified by 2 flash chromatographies on silica gel using Hexanes/EtOAc 10:1 to 2:1 and 1/3 to give 8.20 g (67% yield) of the desired product. ¹H NMR (400 MHz, CDCl₃): δ 3.8-4.0 (m, 2H), 3.73 (s, 3H), 2.75-2.95 (m, 2H), 2.19 (d, 2H), 1.46 (s, 9H), 1.35-1.50 (m, 4H), 0.58-0.70 (m, 1H), 0.37-0.48 (m, 2H), 0.0-0.10 (m, 2H); MS (ESI) m/z: Calculated for C₁₆H₂₇NO₄: 297.2. found: 320 (M+Na)⁺.

### Intermediate VIIIa

### 1-(t-Butoxycarbonyl)-4-(cyclopropylmethyl)piperidine-4-carboxylic acid

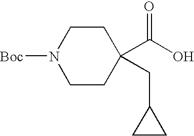

The title compound was prepared according to general procedure E described above in connection with Scheme 2. 1-t-Butyl 4-methyl 4-(cyclopropylmethyl)piperidine-1,4-dicarboxylate (8.20, 27.6 mmol) was dissolved in 60 mL of MeOH and a solution KOH (3.09 g, 55.2 mmol) in 40 ml was added. The reaction mixture was microwaved at 130° C. for 25 min in 5 vials. The methanol was evaporated and the residue was acidified to pH 1-2 with KHSO₄ solid. The acid was extracted with EtOAc (3×50 mL). The combined organic fractions were washed with 1N KHSO₄ and with brine and then dried over Na₂SO₄. The solvent was evaporated to give 7.09 g of the desired acid (91% crude yield). ¹H NMR (400 MHz, CDCl₃): δ 3.82-4.0 (m, 2H), 2.95 (t, 2H), 2.20 (d, 2H), 1.53 (d, 2H), 1.47 (s, 9H), 1.35-1.50 (m, 2H), 0.65-0.78 (m, 1H), 0.40-0.50 (m, 2H), 0.10-0.20 (m, 2H); MS (ESI) m/z: Calculated for C₁₅H₂₅NO₄: 283.2. found: 306 (M+Na)⁺.

### Intermediate IXa

### t-Butyl 4-(3,5-bis(trifluoromethyl)benzylcarbamoyl)-4-(cyclopropylmethyl)piperidine-1-carboxylate

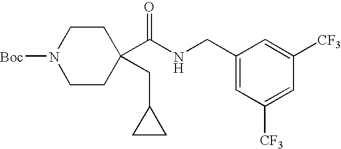

The title compound was prepared according to general procedure A described above. 1-(t-Butoxycarbonyl)-4-(cyclopropylmethyl)piperidine-4-carboxylic acid (7.09 g, 25.0 mmol) and (3,5-bis(trifluoromethyl)phenyl)methanamine (9.51 g, 31.3 mmol) were dissolved in 100 mL of CH₂Cl₂. HOBt monohydrate (3.72 g, 27.5 mmol) and DIEA (13.5 mL, 75.1 mmol) were successively added. EDCI (5.28 g, 27.5 mmol) was added portion-wise and the mixture was stirred for 19 h at RT. The reaction mixture was washed with 1N KHSO₄ and with saturated NaHCO₃. The organic layer was dried over Na₂SO₄ and concentrated in vacuo. The crude amide was purified by flash chromatography on silica gel using 2.5% to 5% MeOH in CH₂Cl₂ as eluent to give 10.4 g (81% yield) of the desired product. ¹H NMR (400 MHz, CDCl₃): δ 7.80 (s, 1H), 7.77 (s, 2H), 6.33 (br s, 1H), 4.62 (d, 2H), 3.7-3.8 (m, 2H), 3.10 (t, 2H), 2.08 (d, 2H), 1.50-1.65 (m, 4H), 1.46 (s, 9H), 0.50-0.65 (m, 1H), 0.34-0.46 (m, 2H), 0.0-0.10 (m, 2H); MS (ESI) m/z: Calculated for C₂₄H₃₀F₆N₂O₃: 508.2. found: 531 (M+Na)⁺.

### Intermediate Xa

### N-(3,5-Bis(trifluoromethyl)benzyl)-4-(cyclopropylmethyl)piperidine-4-carboxamide

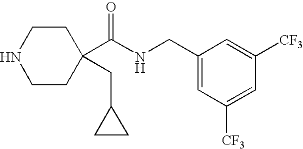

The title compound was prepared according to general procedure A described above. t-Butyl 4-(3,5-bis(trifluoromethyl)benzylcarbamoyl)-4-(cyclopropylmethyl)piperidine-1-carboxylate was dissolved in 50 mL CH₂Cl₂ and 16 ml TFA was added at RT. The reaction mixture was stirred for 17 h and the solvents evaporated. The residue was dissolved in CH₂Cl₂ and the organic layer was washed twice with 50 mL of saturated NaHCO₃ and then dried over Na₂SO₄. The solvent was evaporated to give 8.52 g (quantitative yield) of the desired product. ¹H NMR (400 MHz, CDCl₃): δ 7.81 (s, 1H), 7.79 (s, 2H), 6.33 (t, 1H), 4.63 (d, 2H), 4.38-4.65 (m, 1H), 3.02-3.12 (m, 2H), 2.8-2.9 (m, 2H), 2.12-2.23 (m, 2H), 1.65-1.79 (m, 2H), 1.52 (d, 1H), 0.62-0.51 (m, 2H), 0.36-0.46 (m, 2H), −0.02-0.07 (m, 2H); MS (ESI) m/z: Calculated for C₁₉H₂₂F₆N₂O: 408.2. found: 409 (M+H)⁺.

### Intermediate VIIb

### 1-t-Butyl 3-methyl 3-(cyclopropylmethyl)pyrrolidine-1,3-dicarboxylate

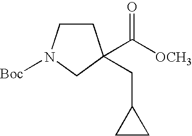

The title compound was prepared according to general procedure D described above in connection with Scheme 2. ¹H NMR (400 MHz, CDCl₃): δ 3.93-3.77 (m, 1H), 3.72 (s, 3H), 3.45-3.22 (m, 3H), 2.45-2.32 (m, 1H), 1.89-1.79 (m, 1H), 1.65-1.59 (m, 2H), 1.48 (s, 9H), 0.67-0.57 (m, 1H), 0.48-0.42 (m, 2H), 0.06-0.01 (m, 2H); MS (ESI) m/z: Calculated for C₁₅H₂₅NO₄: 283.2. found: 306 (M+Na)⁺.

### Intermediate VIIIb

### 1-(t-Butoxycarbonyl)-3-(cyclopropylmethyl)pyrrolidine-3-carboxylic acid

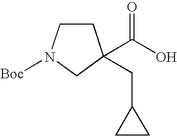

The title compound was prepared according to general procedure E described above in connection with Scheme 2. 1-t-Butyl 3-methyl 3-(cyclopropylmethyl)pyrrolidine-1,3-dicarboxylate (1.00 g, 3.5 mmol.) was dissolved in 2 mL MeOH and a solution KOH (0.59 g, 10.6 mmol.) in 2 mL H₂O was added. The reaction mixture was microwaved at 130° C. for 30 min. The methanol was evaporated and 10 ml of 10% KHSO₄ solution was added to the residue. The acid was extracted with EtOAc (2×20 mL). The combined organic fractions were dried over Na₂SO₄; evaporation of solvent gave 0.98 g of the desired product as an orange oil which crystallized on standing (quantitative yield). ¹H NMR (400 MHz, CDCl₃): δ 3.81-3.93 (m, 1H), 3.36-3.44 (m, 2H), 2.28 (d, 1H), 2.33-2.46 (m, 1H), 1.83-1.91 (m, 1H), 1.60-1.70 (m, 2H), 1.45 (s, 9H), 0.62-0.75 (m, 1H), 0.42-0.50 (m, 2H), 0.05-0.12 (m, 2H); MS (ESI) m/z: Calculated for C₁₄H₂₃NO₄: 269.2. found: 292 (M+Na)⁺.

### Intermediate IXb

### t-Butyl 3-(3,5-bis(trifluoromethyl)benzylcarbamoyl)-3-(cyclopropylmethyl)pyrrolidine-1-carboxylate

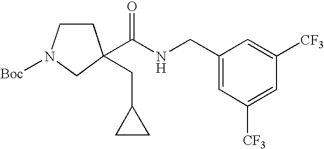

The title compound was prepared according to general procedure A described above. 1-(t-Butoxycarbonyl)-3-(cyclopropylmethyl)pyrrolidine-3-carboxylic acid (0.97 g, 3.53 mmol) and (3,5-bis(trifluoromethyl)phenyl)methanamine (0.901 g, 3.7 mmol) were dissolved in 20 mL of CH₂Cl₂. HOBt monohydrate (0.525 g, 3.9 mmol) and DIEA (1.75 mL, 10.1 mmol) were successively added. EDCI (0.744 g, 3.9 mmol) was added portion-wise and the mixture was stirred for 40 h at RT. The reaction mixture was washed with 1N KHSO₄ and with saturated NaHCO₃. The organic layer was dried over Na₂SO₄ and concentrated in vacuo. The crude amide was purified by flash chromatography on silica gel using 2.5% MeOH in CH₂Cl₂ as eluent to give 1.48 g (85% yield) of the desired product. ¹H NMR (400 MHz, CDCl₃):): δ (as a ≈2:1 mixture of rotamers) 7.78 (bs, 1H), 7.73 (bs, 2H), 6.72 and 6.22 (bs, 1H), 4.58 (bs, 2H), 3.64-3.72 and 3.83-3.94 (2 m, 1H), 3.20-3.50 (m, 3H), 2.16-2.30 and 2.36-2.50 (2 m, 1H), 1.65-2.0 (m, 2H), 1.40-1.50 (m, 1H), 1.41 and 1.43 (2 bs, 9H), 0.50-0.63 (m, 1H), 0.34-0.46 (m, 2H), 0.0-0.08 (m, 2H); MS (ESI) m/z: Calculated for C₂₃H₂₈F₆N₂O₃: 494.2. found: 517 (M+Na)⁺.

### Intermediate Xb

### N-(3,5-Bis(trifluoromethyl)benzyl)-3-(cyclopropylmethyl)pyrrolidine-3-carboxamide

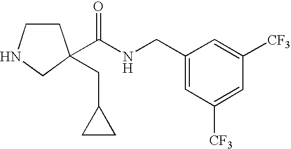

The title compound was prepared according to general procedure B described above. t-Butyl 3-(3,5-bis(trifluoromethyl)benzylcarbamoyl)-3-(cyclopropylmethyl)pyrrolidine-1-carboxylate was dissolved in 10 mL CH₂Cl₂ and 2.3 ml TFA was added at RT. The reaction mixture was stirred for 15 h and the solvents evaporated. The residue was dissolved in CH₂Cl₂ and the organic layer was washed twice with saturated NaHCO₃ and then dried over Na₂SO₄. The solvent was evaporated to give 1.06 g (90% yield) of the desired product. ¹H NMR (400 MHz, CDCl₃): δ 8.24 (bs, 1H), 7.76 (bs, 1H), 7.72 (bs, 2H), 4.48-4.62 (m, 2H), 3.43 (d, 1H), 2.96-3.13 (m, 2H), 2.85 (d, 1H), 2.23 (dd, 1H), 1.95-2.11 (m, 2H), 1.75-1.85 (m, 1H), 1.15 (dd, 1H), 0.62-0.75 (m, 1H), 0.35-0.46 (m, 2H), 0.02-0.14 (m, 2H); MS (ESI) m/z: Calculated for C₁₈H₂₀F₆N₂O: 394.2. found 395 (M+H)⁺.

### Example 1

### N-(3,5-Bis(trifluoromethyl)benzyl)-1-((tetrahydro-2H-pyran-4-yl)methyl)-4-methylpiperidine-4-carboxamide

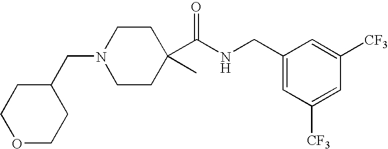

The title compound was prepared according to general procedure C described above in connection with Scheme 1. N-(3,5-Bis(trifluoromethyl)benzyl)-4-methylpiperidine-4-carboxamide 2,2,2-trifluoroacetate was dissolved in DCM and extracted with a saturated solution of NaHCO₃. The organic solvent was removed under vacuum to obtain free amine N-(3,5-bis(trifluoromethyl)benzyl)-4-methylpiperidine-4-carboxamide. The free amine (0.12 g, 0.33 mmol), 4-formyltetrahydropyran (0.04 g, 0.33 mmol) and NaBH(OAc)₃ (0.10 g, 0.49 mmol) were dissolved in 5 mL of DCM and stirred for 16 h at RT. The reaction was washed with a saturated solution of NaHCO₃. The DMS was evaporated and the crude residue was purified by silica chromatography in 10% EtOAc/hexanes to obtain de desired product (48 mg, 32% yield): ¹H NMR (400 MHz, MeOH-d₄): δ 7.79 (s, 1H), 7.73 (s, 2H), 6.13 (t, 1H), 4.59 (d, 2H) 3.95 (dd, 2H), 3.38 (t, 2H), 2.54 (m, 2H), 2.22 (t, 2H), 2.14 (d, 2H), 2.03 (m, 2H), 1.73 (m, 1H), 1.59 (m, 4H), 1.27 (m, 2H), 1.23 (s, 3H); MS (ESI) m/z: Calculated for C₂₂H₂₈F₆N₂O₂: 466.2. found: 467.1 (M+H)⁺.

### Example 2

### N-(3,5-Bis(trifluoromethyl)benzyl)-4-(cyclopropylmethyl)-1-(4-hydroxy-3-methoxybenzyl)piperidine-4-carboxamide

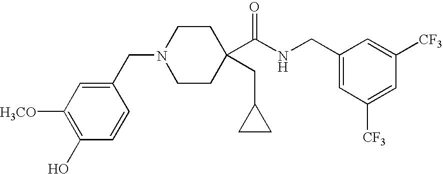

The title compound was prepared according to procedure C described above in connection with Scheme 1. A mixture of vanillin (0.99 mmol), and N-(3,5-Bis(trifluoromethyl)benzyl)-4-(cyclopropylmethyl)piperidine-4-carboxamide (0.24 mmol) in DCM (5 mL) was stirred at room temperature for 1 h. Sodium triacetoxyborohydride (0.71 mmol) was added and the reaction mixture was stirred for 16 h at RT. After concentration of solvent under reduced pressure, the resulting residue was dissolved in ethyl acetate, then washed with water and brine. The organic extract was dried, filtered and concentrated. The crude product was purified by reverse phase preparative HPLC (Phenomenex reverse phase Luna 5u C18(2) column, 60×21.2 mm ID, mobile phase: A=0.05% TFA in water; B=0.05% TFA in acetonitrile). The flow rate was 18 mL/min) to yield the desired final product with purity greater than 95%. (21.0 mg TFA-salt, 13% yield). ¹H NMR (400 MHz, MeOH-d₄): δ 8.79 (m, 1H), 8.02 (s, 2H), 7.97 (s, 1H), 7.10 (m, 1H), 6.95 (m, 2H), 4.64 (d, 2H), 4.24 (s, 2H), 3.96 (s, 3H), 3.50 (m, 2H), 3.02 (m, 2H), 2.58 (m, 2H), 1.79 (m, 2H), 1.55 (d, 2H), 0.53 (m, 1H), 0.42 (m, 2H), 0.00 (m, 2H); MS (ESI) m/z: Calculated for C₂₇H₃₀F₆N₂O₃: 544.2. found 545.1 (M+H⁺).

### Example 3

### (rac) N-(3,5-Bis(trifluoromethyl)benzyl)-3-(cyclopropylmethyl)-1-(4-hydroxy-3-methoxybenzyl)pyrrolidine-3-carboxamide

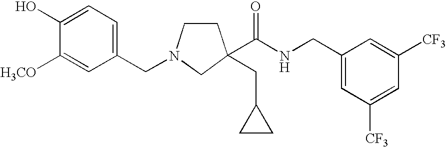

The title compound was prepared according to procedure C described above in connection with Scheme 1. N-(3,5-Bis(trifluoromethyl)benzyl)-3-(cyclopropylmethyl)pyrrolidine-3-carboxamide (142 mg, 0.36 mmol) and vanillin (55 mg, 0.36 mmol) were dissolved in 2.5 mL CH₂Cl₂. NaBH(OAc)₃ (76.3 mg, 0.50 mmol) was added and the reaction mixture was stirred for 8 h at RT. The solvent was evaporated and the residue dissolved in EtOAc and was washed 5 mL 1N NaOH and with brine. The organic layer was dried over Na₂SO₄ and concentrated in vacuo. The crude product was purified by flash chromatography on silica gel using a gradient of 1 to 5% MeOH in CH₂Cl₂ to give 110 mg (58% yield) of the desired product. ¹H NMR (400 MHz, CDCl₃): δ 8.58 (t, 1H), 7.76 (bs, 1H), 7.67 (bs, 2H), 6.75 (d, 1H), 6.62-6.67 (m, 2H), 4.52 (d, 2H), 3.70 (s, 3H), 3.62 (d, 1H), 3.51 (d, 1H), 3.19 (d, 1H), 3.0-3.08 (m, 1H), 2.24-2.40 (m, 3H), 2.12-2.13 (m, 1H), 1.80-1.89 (m, 1H), 1.10 (dd, 1H), 0.60-0.72 (m, 2H), 0.35-0.44 (m, 2H), 0.02-0.13 (m, 2H); MS (ESI) m/z: Calculated for C₂₆H₂₈F₆N₂O₃: 530.2. found 531 (M+H)⁺.

### Example 4

### N-(3,5-Bis(trifluoromethyl)benzyl)-1-((benzo[d][1,3]dioxol-5-yl)methyl)-4-methylpiperidine-4-carboxamide

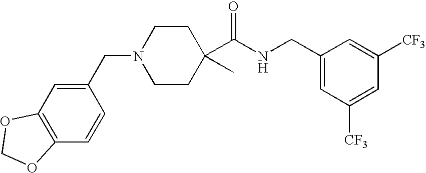

The title compound was prepared according to general procedures described above in connection with Scheme 1 (50 mg, 35% yield): ¹H NMR (400 MHz, MeOH-d₄): δ 7.79 (s, 1H), 7.73 (s, 2H), 6.84 (s, 1H), 6.73 (s, 21H), 6.16 (bs, 1H), 5.94 (s, 2H), 4.59 (d, 2H), 3.37 (s, 2H), 2.58 (m, 2H), 2.23 (t, 2H), 2.04 (d, 2H), 1.60 (t, 2H), 1.22 (s, 3H); MS (ESI) m/z: Calculated for C₂₄H₂₄F₆N₂O₃: 502.2. found: 503.1 (M+H)⁺.

### Example 5

### 1-(Benzo[d][1,3]dioxol-5-ylmethyl)-N-(3,4-dichlorobenzyl)-4-methylpiperidine-4-carboxamide

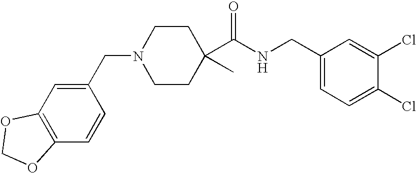

The title compound was prepared according to general procedures described above in connection with Scheme 1 (0.21 g, 80% yield): ¹H NMR (400 MHz, CDCl₃): δ 7.40-7.38 (m, 2H), 7.18-7.16 (m, 1H), 6.83 (s, 1H), 6.73 (s, 2H), 5.93 (s, 2H), 4.41 (d, 2H), 3.39 (s, 2H), 2.55-2.52 (m, 2H), 2.29-2.25 (m, 2H), 2.04-1.98 (m, 2H), 1.69-1.54 (m, 2H), 1.20 (s, 3H); MS (ESI) m/z: Calculated for C₂₂H₂₄Cl₂N₂O₃: 434.1. found: 435.2 (M+H)⁺.

### Example 6

### N-(3,4-Dichlorobenzyl)-4-methyl-1-((tetrahydro-2H-pyran-4-yl)methyl)piperidine-4-carboxamide

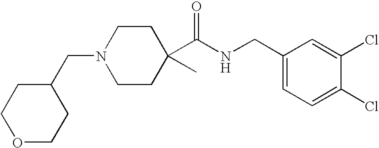

The title compound was prepared according to the general procedures described above in connection with Scheme 1 (0.195 g, 79% yield): ¹H NMR (400 MHz, CDCl₃): δ 7.40-7.35 (m, 2H), 7.12-7.09 (m, 1H), 4.41 (d, 2H), 3.97-3.93 (m, 2H), 3.40-3.34 (m, 2H), 2.60-1.25 (m, 15H), 1.23 (s, 3H); MS (ESI) m/z: Calculated for C₂₀H₂₈Cl₂N₂O₂: 398.2. found: 399.3 (M+H)⁺.

### Example 7

### 1-(Benzo[c][1,2,5]oxadiazol-5-ylmethyl)-N-(3,4-dichlorobenzyl)-4-methylpiperidine-4

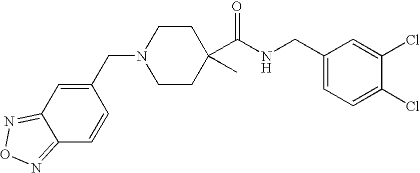

The title compound was prepared according to the general procedures described above in connection with Scheme 1 (0.192 g, 72% yield): ¹H NMR (400 MHz, CDCl₃): δ 7.77-7.67 (m, 2H), 7.48-7.36 (m, 3H), 7.12-7.10 (m, 1H), 5.98 (bs, 1H), 4.43 (d, 2H), 3.54 (s, 2H), 2.58-2.56 (m, 2H), 2.36-2.30 (m, 2H), 2.05-2.00 (m, 2H), 1.63-1.60 (m, 2H), 1.23 (s, 3H); MS (ESI) m/z: Calculated for C₂₁H₂₂Cl₂N₄O₂: 432.1. found: 433.2 (M+H)⁺.

### Example 8

### (4-Methyl-1-((tetrahydro-2H-pyran-4-yl)methyl)piperidin-4-yl)(4-(3-(trifluoromethyl)phenyl)piperazin-1-yl)methanone

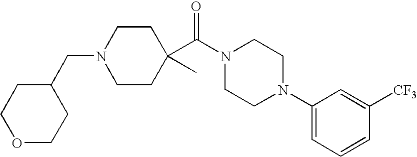

The title compound was prepared according to the general procedures described above in connection with Scheme 1 (3.7 mg, 8% yield): ¹H NMR (400 MHz, CDCl₃): δ 7.40 (t, 1H), 7.17 (d, 1H), 7.12 (s, 1H), 7.09 (d, 1H), 3.97 (dd, 2H), 3.83 (s, 3H), 3.59 (d, 2H), 3.38 (t, 2H), 3.23 (s, 3H), 2.85 (m, 8H), 2.36 (d, 2H), 2.16 (m, 3H), 1.75 (d, 2H), 1.42 (s, 3H); MS (ESI) m/z: Calculated for C₂₄H₃₄F₃N₃O₂: 453.3. found: 454.3 (M+H)⁺.

### Example 9

### 4-Methyl-1-((tetrahydro-2H-pyran-4-yl)methyl)-N-(3-(trifluoromethyl)benzyl)piperidine-4-carboxamide

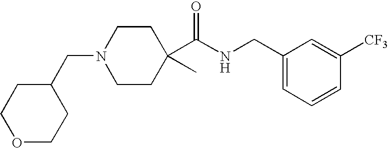

The title compound was prepared according to the general procedures described above in connection with Scheme 1 (4.8 mg, 12% yield): ¹H NMR (400 MHz, CDCl₃): δ 7.54 (d, 1H), 7.51 (s, 1H), 7.47 (d, 1H), 7.19 (m, 1H), 4.49 (d, 2H), 3.94 (dd, 2H), 3.48 (d, 2H), 3.35 (t, 2H), 2.91 (m, 2H), 2.75 (br s, 2H), 2.27 (d, 2H), 2.04 (t, 2H), 1.92 (br s, 2H), 1.68 (d, 2H), 1.36 (m, 1H), 1.29 (s, 3H); MS (ESI) m/z: Calculated for C₂₁H₂₉F₃N₂O₂: 398.2. found: 399.3 (M+H)⁺.

### Example 10

### N-(3-Fluoro-5-(trifluoromethyl)benzyl)-4-methyl-1-((tetrahydro-2H-pyran-4-yl)methyl)piperidine-4-carboxamide

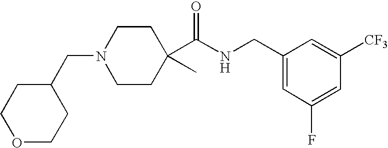

The title compound was prepared according to the general procedures described above in connection with Scheme 1 (2.5 mg, 6% yield): ¹H NMR (400 MHz, CDCl₃): δ 7.32 (s, 1H), 7.25-7.20 (m, 1H), 7.18 (d, 1H), 4.48 (d, 2H), 3.95 (d, 2H), 3.49 (d, 2H), 3.36 (t, 2H), 2.89-2.85 (m, 2H), 2.77 (br s, 2H), 2.27 (br s, 2H), 2.07 (m, 2H), 1.68 (m, 5H), 1.31 (s, 3H); MS (ESI) m/z: Calculated for C₂₁H₂₈F₄N₂O₂: 416.2. found: 417.3 (M+H)⁺.

### Example 11

### N-(3,5-Bis(trifluoromethyl)benzyl)-1-((tetrahydro-2H-pyran-4-yl)methyl)-4-(methoxymethyl)piperidine-4-carboxamide

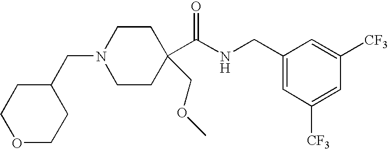

The title compound was prepared according to the general procedures described above in connection with Scheme 2 (50 mg, 72% yield): ¹H NMR (400 MHz, MeOH-d₄): δ 7.89 (s, 2H), 7.83 (s, 1H), 4.56 (s, 2H), 3.92 (dd, 2H), 3.44 (m, 5H), 2.94 (bs, 2H), 2.45 (bs, 4H), 2.22 (d, 2H), 1.90 (bs, 1H), 1.68 (m, 4H), 1.24 (dt, 4H); MS (ESI) m/z: Calculated for C₂₃H₃₀F₆N₂O₃: 496.2. found: 497.3 (M+H)⁺.

### Example 12

### N-(3,5-Bis(trifluoromethyl)benzyl)-4-isobutyl-1-((tetrahydro-2H-pyran-4-yl)methyl)piperidine-4-carboxamide

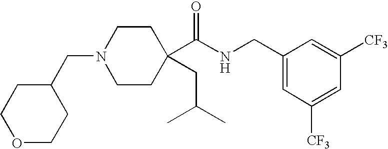

The title compound was prepared according to the general procedures described above in connection with Scheme 2 (66 mg, 33% yield): ¹H NMR (400 MHz, MeOH-d₄): δ 7.92 (s, 2H), 7.85 (s, 1H), 4.50 (s, 2H), 3.91 (dd, 2H), 3.39 (t, 2H), 2.69 (d, 2H), 2.14 (m, 4H), 2.04 (t, 2H), 1.77 (m, 1H), 1.55 (m, 5H), 1.44 (d, 2H), 1.22 (dq, 2H), 0.80 (dd, 6H); MS (ESI) m/z: Calculated for C₂₅H₃₄F₆N₂O₂: 508.3. found: 509.4 (M+H)⁺.

### Example 13

### N-(3,5-Bis(trifluoromethyl)benzyl)-4-(pyridin-4-ylmethyl)-1-((tetrahydro-2H-pyran-4-yl)methyl)piperidine-4-carboxamide

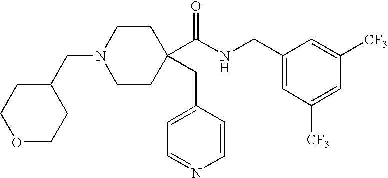

The title compound was prepared according to the general procedures described above in connection with Scheme 2 (40.0 mg, 27% yield): ¹H NMR (400 MHz, CDCl₃): δ 9.34 (s, 1H), 8.72 (s, 2H), 7.89 (s, 2H), 7.78 (m, 3H), 4.52 (d, 2H), 3.91 (d, 2H), 3.55 (d, 2H), 3.34 (t, 4H), 2.70 (m, 6H), 2.46 (m, 2H), 2.07 (m, 1H), 1.78 (d, 21-I), 1.36 (q, 2H); MS (ESI) m/z: Calculated for C₂₇H₃₁F₆N₃O₂: 543.2. found: 544.3 (M+H)⁺.

### Example 14

### 4-Benzyl-N-(3,5-bis(trifluoromethyl)benzyl)-1-((tetrahydro-2H-pyran-4-yl)methyl)piperidine-4-carboxamide

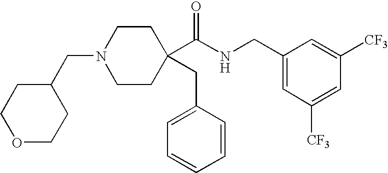

The title compound was prepared according to the general procedures described above in connection with Scheme 2 (5.00 mg, 4% yield): ¹H NMR (400 MHz, CDCl₃): δ 7.77 (s, 1H), 7.64 (s, 2H), 7.20 (m, 3H), 7.02 (m, 2H), 5.54 (t, 1H), 4.39 (d, 2H), 3.95 (dd, 2H), 3.37 (dt, 2H), 2.82 (s, 2H), 2.71 (d, 2H), 2.14 (d, 2H), 2.04 (d, 2H), 1.71 (t, 2H), 1.60 (m, 3H), 1.25 (m, 4H); MS (ESI) m/z: Calculated for C₂₈H₃₂F₆N₂O₂: 542.2. found: 543.3 (M+H)⁺.

### Example 15

### N-(3,5-Bis(trifluoromethyl)benzyl)-4-(4-methoxybenzyl)-1-((tetrahydro-2H-pyran-4-yl)methyl)piperidine-4-carboxamide

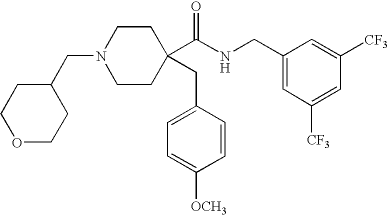

The title compound was prepared according to the general procedures described above in connection with Scheme 2 (6.00 mg, 5% yield): ¹H NMR (400 MHz, CDCl₃): δ 7.78 (s, 1H), 7.68 (s, 2H), 6.94 (d, 2H), 6.73 (d, 2H), 5.64 (t, 1H), 4.42 (d, 2H), 3.95 (dd, 2H), 3.76 (s, 3H), 3.37 (t, 2H), 2.76 (s, 2H), 2.72 (d, 2H), 2.14 (d, 2H), 2.03 (d, 4H), 1.66 (m, 3H), 1.25 (m, 4H); MS (ESI) m/z: Calculated for C₂₉H₃₄F₆N₂O₃: 572.3. found: 573.4 (M+H)⁺.

### Example 16

### N-(3,5-Bis(trifluoromethyl)benzyl)-1-(tetrahydro-2H-pyran-4-yl)methyl)-4-(thiophene-3-ylmethyl)piperidine-4-carboxamide

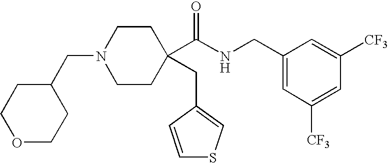

The title compound was prepared according to the general procedures described above in connection with Scheme 2 (30.0 mg, 17% yield): ¹H NMR (400 MHz, MeOH-d₄): δ 8.70 (br d, 1H), 8.89-7.80 (m, 3H), 7.21-7.19 (m, 1H), 6.90-6.88 (m, 1H), 6.73 (d, 1H), 4.57 (d, 2H), 3.90-3.88 (m, 2H), 3.60-3.51 (m, 4H), 3.10-2.88 (m, 6H), 2.41-2.39 (m, 2H), 2.20-1.20 (m, 7H); MS (ESI) m/z: Calculated for C₂₆H₃₀F₆N₂O₂S: 548.2. found: 549.3 (M+H)⁺.

### Example 17

### N-(3,5-Bis(trifluoromethyl)benzyl)-4-(pyridin-3-ylmethyl)-1-((tetrahydro-2H-pyran-4-yl)methyl)piperidine-4-carboxamide

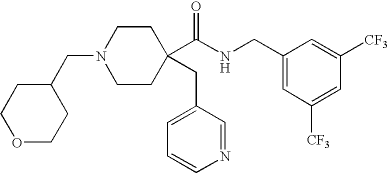

The title compound was prepared according to the general procedures described above in connection with Scheme 2 (9.5 mg, 16.8% yield): ¹H NMR (400 MHz, CDCl₃): δ 8.80 (s, 1H), 8.51 (d, 1H), 7.96 (d, 1H), 7.80-7.78 (m, 3H), 7.66 (t, 1H), 4.51 (d, 2H), 3.95 (d, 2H), 3.52 (d, 2H), 3.46 (d, 2H), 3.16 (s, 2H), 2.71 (d, 2H), 2.34 (m, 2H), 2.11 (t, 2H), 1.80 (t, 2H), 1.66-1.58 (m, 5H); MS (ESI) m/z: Calculated for C₂₇H₃₁F₆N₃O₂: 543.2. found: 544.3 (M+H)⁺.

### Example 18

### N-(3,5-Bis(trifluoromethyl)benzyl)-4-isobutyl-1-(2-(piperidin-1-yl)ethyl)piperidine-4-carboxamide

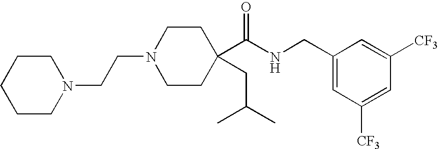

The title compound was prepared according to the general procedures described above in connection with Scheme 2, using procedure F in the final step: a mixture of intermediate Xa (115 mg, 0.22 mmol), 1-(2-chloroethyl)piperidine hydrochloride salt (49 mg, 0.26 mmol), DIEA (85 uL, 0.48 mmol) and potassium carbonate (76 mg, 0.55 mmol) in DMF (3 mL) was irradiated in a microwave instrument at 100° C. for 30 min (Personal Chemistry Emrys™ Optimizer microwave reactor). The reaction mixture was cooled and diluted with ethyl acetate. The organic layer was washed with brine (×3), then dried over Na₂SO₄, filtered and concentrated under reduced pressure. The crude product was purified by reverse phase preparative HPLC to yield the desired final product as trifluoroacetic salt with purity greater than 95% (13 mg, 11.3% yield): ¹H NMR (400 MHz, MeOH-d₄): δ 8.70 (t, 1H, NH), 7.94 (s, 2H), 7.88 (s, 1H), 4.54 (d, 2H), 3.55 (m, 7H), 3.55-3.30 (m, 3H), 3.07 (br s, 2H), 2.39 (d, 2H), 1.88 (m, 6H), 1.69 (s, 2H), 1.53 (m, 3H), 0.77 (d, 6H); MS (ESI) m/z: Calculated for C₂₆H₃₇F₆N₃O: 521.3. found: 522.3 (M+H)⁺.

### Example 19

### N-(3,5-Bis(trifluoromethyl)benzyl)-4-isobutyl-1-(pyridin-2-yl)piperidine-4-carboxamide

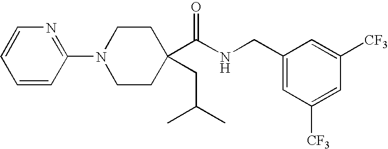

The title compound was prepared according to the general procedures described above in connection with Scheme 2, using procedure G in the final step. N-(3,5-Bis(trifluoromethyl)benzyl)-4-isobutylpiperidine-4-carboxamide trifluoroacetic acid salt (120 mg, 0.23 mmol), 2-bromopyridine (44 mg, 0.276 mmol), Pd(dppf)Cl₂ (5.6 mg, 0.007 mmol), dppf (5.7 mg, 0.01 mmol), triethylamine (40 uL, 0.276 mmol) and sodium tert-butoxide (44 mg, 0.46 mmol) was mixed in toluene (1.2 mL). The mixture was heated at 120° C. for 30 min under microwave irradiation. Upon completion of the reaction, the reaction mixture was concentrated under reduced pressure. The residues was diluted with ethyl acetate, and then washed with NH₄Cl, water and brine. The crude product was purified by reverse phase preparative HPLC to yield the desired final product as trifluoroacetic salt (6.1 mg, 6.7% yield) with purity greater than 95%: ¹H NMR (400 MHz, MeOH-d₄): δ 8.68 (t, 1H, NH), 7.99 (t, 1H), 7.94 (s, 2H), 7.90-7.87 (m, 2H), 7.36 (d, 1H), 6.94 (t, 1H), 4.54 (d, 2H), 3.95 (d, 2H), 3.37 (t, 2H), 2.34 (d, 2H), 1.69 (t, 2H), 1.62-1.55 (m, 3H), 0.81 (d, 6H); MS (ESI) m/z: Calculated for C₂₄H₂₇F₆N₃O: 487.2. found: 488.3 (M+H)⁺.

### Example 20

### N-(3,5-Bis(trifluoromethyl)benzyl)-1-(furan-2-ylmethyl)-4-isobutylpiperidine-4-carboxamide

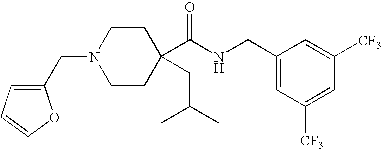

The title compound was prepared according to the general procedures described above in connection with Scheme 2 (14.4 mg TFA-salt, 17% yield): MS (ESI) m/z: Calculated for C₂₄H₂₈F₆N₂O₂: 490.2. found 491.3 (M+H)⁺.

### Example 21

### 1-(Benzo[d][1,3]dioxol-5-ylmethyl)-N-(3,5-bis(trifluoromethyl)benzyl)-4-isobutylpiperidine-4-carboxamide

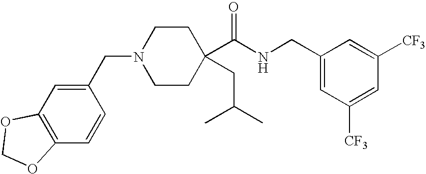

The title compound was prepared according to the general procedures described above in connection with Scheme 2 (6.3 mg TFA-salt, 7% yield): ¹H NMR (400 MHz, MeOH-d₄): δ 8.69 (m, 1H), 7.94 (s, 2H), 7.90 (s, 1H), 6.93 (m, 3H), 6.02 (s, 2H), 4.54 (d, 2H), 4.17 (s, 2H), 3.41 (m, 2H), 2.96 (m, 2H), 2.43 (m, 2H), 1.64 (m, 2H), 1.52 (m, 1H), 1.47 (d, 2H), 0.75 (d, 6H); (MS (ESI) m/z: Calculated for C₂₇H₃₀F₆N₂O₃: 544.2. found 545.2 (M+H)⁺.

### Example 22

### 1-(Benzo[d][1,3]dioxol-5-ylmethyl)-N-(3,5-bis(trifluoromethyl)benzyl)-4-(pyridin-3-ylmethyl)piperidine-4-carboxamide

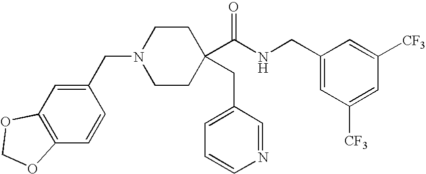

The title compound was prepared according to the general procedures described above in connection with Scheme 2 (6.9 mg, 8% yield): ¹H NMR (400 MHz, MeOH-d₄): δ 8.78 (br s, 1H, NH), 8.55 (s, 1H), 8.42 (s, 1H), 7.96-7.94 (m, 3H), 7.71 (d, 1H), 7.49 (t, 1H), 6.92-6.87 (m, 3H), 6.01 (m, 2H), 4.54 (d, 2H), 4.15 (d, 2H), 3.45 (d, 2H), 2.98 (s, 2H), 2.88 (t, 2H), 2.39 (d, 2H), 1.82 (t, 2H); MS (ESI) m/z: Calculated for C₂₉H₂₇F₆N₃O₃: 579.2. found: 580.2 (M+H)⁺.

### Example 23

### 1-(Benzo[d][1,3]dioxo-5-ylmethyl)-N-(3,5-bis(trifluoromethyl)benzyl)-4-(thiophene-3-ylmethyl)piperidine-4-carboxamide

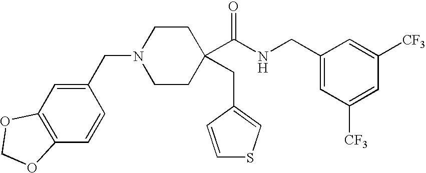

The title compound was prepared according to the general procedures described above in connection with Scheme 2 (28.0 mg, 26% yield): ¹H NMR (400 MHz, CDCl₃): δ 7.80-7.65 (m, 3H), 7.20-7.18 (m, 1H), 6.91-6.89 (m, 2H), 6.78 (m, 3H), 6.00 (m, 2H), 4.44 (d, 2H), 3.50-3.40 (m, 2H), 2.98 (s, 2H), 2.80-2.60 (m, 4H), 1.60-1.52 (m, 4H); MS (ESI) m/z: Calculated for C₂₈H₂₆F₆N₂O₃S: 584.2. found: 585.2 (M+H)⁺.

### Example 24

### 1-(Benzo[d][1,3]dioxol-5-ylmethyl)-N-(3,5-bis(trifluoromethyl)benzyl)-4-(pyridin-4-ylmethyl)piperidine-4-carboxamide

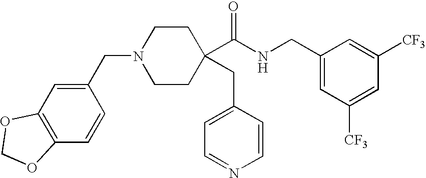

The title compound was prepared according to the general procedures described above in connection with Scheme 2 (28.0 mg, 15% yield): ¹H NMR (400 MHz, CDCl₃): δ 8.43 (d, 2H), 7.81-7.70 (m, 3H), 6.96-6.71 (m, 5H), 5.94 (s, 2H), 5.80 (br s, 1H), 4.46 (d, 2H), 3.37-3.35 (m, 2H), 2.83 (s, 2H), 2.73-2.70 (m, 2H), 2.18-1.99 (m, 4H), 1.75-1.65 (m, 2H); MS (ESI) m/z: Calculated for C₂₉H₂₇F₆N₃O₃: 579.2. found: 580.2 (M+H)⁺.

### Example 25

### 1-(Benzo[d][1,3]dioxol-5-ylmethyl)-4-isobutyl-N-(3-(trifluoromethyl)benzyl)piperidine-4-carboxamide

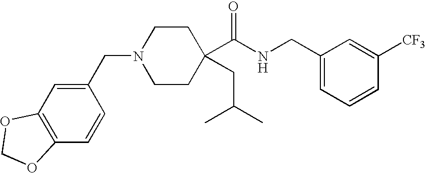

The title compound was prepared according to the general procedures described above in connection with Scheme 2 (10.4 mg, 11% yield): ¹H NMR (400 MHz, MeOH-d₄): δ 8.59 (br s, 1H, NH), 7.64 (s, 1H), 7.59-7.51 (m, 3H), 6.95-6.89 (m, 3H), 6.02 (s, 2H), 4.46 (s, 2H), 4.16 (s, 2H), 3.39 (d, 2H), 2.92 (t, 2H), 2.44 (d, 2H), 2.03 (br s, 1H), 1.63 (t, 2H), 1.48 (br s, 2H) 0.79 (d, 6H); MS (ESI) m/z: Calculated for C₂₆H₃₁F₃N₂O₃: 476.2. found: 477.2 (M+H)⁺.

### Example 26

### 1-(4-(Benzo[d][1,3]dioxol-5-yl)-4-hydroxycyclohexyl)-N-(3,5-bis(trifluoromethyl)benzyl)-4-isobutylpiperidine-4-carboxamide

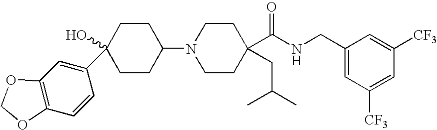

The title compound was prepared according to the general procedures described above in connection with Scheme 2. A mixture of 4-(benzo[d][1,3]dioxol-5-yl)-4-hydroxycyclohexanone (44.7 mg, 0.19 mmol), N-(3,5-bis(trifluoromethyl)benzyl)-4-isobutylpiperidine-4-carboxamide trifluoroacetic acid salt (100 mg, 0.191 mmol), triethyl amine (160 uL, 1.15 mmol) in THF (4 mL) was stirred at room temperature for 0.5 h. Sodium triacetoxyborohydride (121 mg, 0.576 mmol) was added and the reaction mixture was stirred for 16 h at room temperature. After concentration of solvent under reduced pressure, the resulting residue was dissolved in ethyl acetate, then washed with water and brine. The organic extract was dried, filtered and concentrated. The crude product was obtained as a mixture of two cis/trans isomers (ca. 1:1 ratio) which were further separated by reverse phase preparative HPLC to yield isomer A (eluented at 6.09 min) and isomer B (eluented at 6.27 min).

Isomer A (7.2 mg): ¹H NMR (400 MHz, MeOH-d₄): δ 8.59 (t, 1H), 7.92 (m, 3H), 7.08 (m, 2H), 6.82 (d, 1H), 5.94 (m, 2H), 4.51 (d, 2H), 3.46 (d, 2H), 3.24 (m, 2H), 2.82 (t, 2H), 2.53 (d, 2H), 2.40 (d, 2H), 2.21 (m, 1H), 2.05 (d, 2H), 1.76 (t, 2H), 1.67-1.45 (m, 6H), 0.79 (d, 6H); MS (ESI) m/z: Calculated for C₃₂H₃₈F₆N₂O₄: 628.3. found: 629.2 (M+H)⁺.

Isomer B (7.7 mg): ¹H NMR (400 MHz, MeOH-d₄): δ 8.75 (m, 1H) 7.95-7.90 (m, 3H), 6.99 (s, 1H), 6.94 (d, 1H), 6.76 (d, 1H), 5.91 (s, 2H), 4.47 (s, 2H), 3.28 (d, 2H), 3.20 (m, 2H), 2.98 (t, 2H), 2.49 (d, 2H), 2.09-1.90 (m, 8H), 1.71 (m, 2H), 1.57 (m, 1H), 1.52 (m, 2H), 0.79 (d, 6H); MS (ESI) m/z: Calculated for C₃₂H₃₈F₆N₂O₄: 628.3. found: 629.2 (M+H)⁺.

### Example 27

### 1-(Benzo[d][1,3]dioxol-5-ylmethyl)-N-(3,5-bis(trifluoromethyl)benzyl)-4-((tetrahydro-2H-pyran-4-yl)methyl)piperidine-4-carboxamide

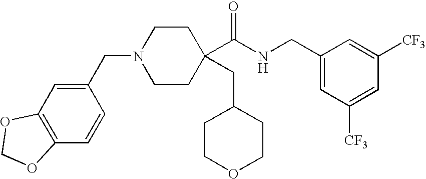

The title compound was prepared according to the general procedures described above in connection with Scheme 2 (3.0 mg, 12% yield): ¹H NMR (400 MHz, MeOH-d₄): δ 7.96 (s, 2H), 7.92 (s, 1H), 6.95 (s, 1H), 6.91 (m, 2H), 6.02 (s, 2H), 4.52 (s, 2H), 4.17 (s, 2H), 3.68 (d, 2H), 3.40 (d, 2H), 3.12 (t, 2H), 2.95 (t, 2H), 2.42 (d, 2H), 1.65 (t, 2H), 1.47 (2H), 1.39 (s, 1H), 1.32 (d, 2H), 1.10 (m, 2H); MS (ESI) m/z: Calculated for C₂₉H₃₂F₆N₂O₄: 586.2. found: 587.2 (M+H)⁺.

### Example 28

### 1-(Benzo[d][1,3]dioxol-5-ylmethyl)-N-(3,5-bis(trifluoromethyl)benzyl)-4-(4-methoxybenzyl)piperidine-4-carboxamide

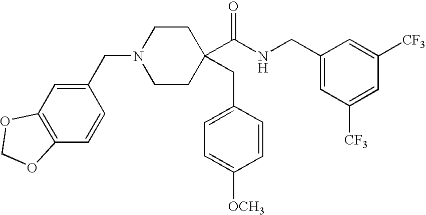

The title compound was prepared according to the general procedure described above in connection with Scheme 2 (4.0 mg, 1% yield). ¹H NMR (400 MHz, MeOH-d₄): δ 8.58 (s, 1H), 8.00-7.88 (m, 3H), 6.92-6.62 (m, 7H), 6.01 (s, 2H), 4.52 (s, 2H), 4.14 (s, 2H), 3.70 (s, 3H), 3.43-3.30 (m, 2H), 2.92-2.85 (m, 2H), 2.75 (s, 2H), 2.37-2.34 (m, 2H), 1.77-1.70 (m, 2H); MS (ESI) m/z: Calculated for C₃₁H₃₀F₆N₂O₄: 608.2. found: 609 (M+H)⁺.

### Example 29

### 1-(Benzo[d][1,3]dioxol-4-ylmethyl)-N-(3,5-bis(trifluoromethyl)benzyl)-4-isobutylpiperidine-4-carboxamide

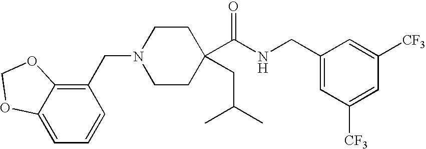

The title compound was prepared according to the general procedures described above in connection with Scheme 2 (31 mg TFA-salt, 20% yield): ¹H NMR (400 MHz, MeOH-d₄): δ 8.70 (m, 1H), 7.93 (s, 2H), 7.89 (s, 1H), 6.92 (m, 3H), 6.03 (s, 2H), 4.52 (d, 2H), 4.23 (s, 2H), 3.48 (m, 2H), 3.00 (m, 2H), 2.42 (m, 2H), 1.68 (m, 2H), 1.53 (m, 1H), 1.47 (d, 2H), 0.76 (d, 6H); MS (ESI) m/z: Calculated for C₂₇H₃₀F₆N₂O₃: 544.2. found 545.3 (M+H)⁺.

### Example 30

### N-(3,5-Bis(trifluoromethyl)benzyl)-1-(3-hydroxy-4-methoxybenzyl)-4-isobutylpiperidine-4-carboxamide

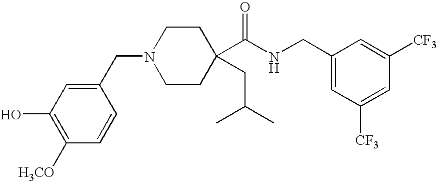

The title compound was prepared according to the general procedures described above in connection with Scheme 2 (72 mg TFA-salt, 57% yield): ¹H NMR (400 MHz, MeOH-d₄): δ 8.69 (m, 1H), 7.94 (s, 2H), 7.89 (s, 1H), 6.98 (m, 1H), 6.89 (m, 2H), 4.54 (d, 2H), 4.12 (s, 2H), 3.88 (s, 3H), 3.39 (m, 2H), 2.94 (m, 2H), 2.42 (m, 2H), 1.63 (m, 2H), 1.52 (m, 1H), 1.46 (d, 2H), 0.74 (d, 6H); MS (ESI) m/z: Calculated for C₂₇H₃₂F₆N₂O₃: 546.2. found 547.2 (M+H)⁺.

### Example 31

### N-(3,5-Bis(trifluoromethyl)benzyl)-1-(4-hydroxy-3-methoxybenzyl)-4-isobutylpiperidine-4-carboxamide

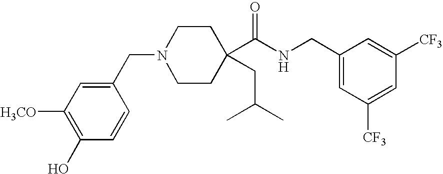

The title compound was prepared according to the general procedures described above in connection with Scheme 2 (34 mg TFA-salt, 27% yield): ¹H NMR (400 MHz, MeOH-d₄): δ 8.70 (m, 1H), 7.94 (s, 2H), 7.89 (s, 1H), 7.02 (m, 1H), 6.87 (m, 2H), 4.53 (d, 2H), 4.15 (s, 2H), 3.88 (s, 3H), 3.40 (m, 2H), 2.94 (m, 2H), 2.43 (m, 2H), 1.67 (m, 2H), 1.53 (m, 1H), 1.47 (d, 2H), 0.75 (d, 6H); MS (ESI) m/z: Calculated for C₂₇H₃₂F₆N₂O₃: 546.2. found 547.1 (M+H)⁺.

### Example 32

### 1-(Benzo[d][1,3]dioxol-5-ylmethyl)-4-benzyl-N-(3,5-bis(trifluoromethyl)benzyl)piperidine-4-carboxamide

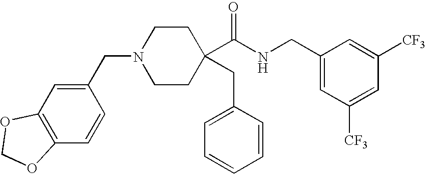

The title compound was prepared according to the general procedures described above in connection with Scheme 2 (5.0 mg, 1.1% yield): ¹H NMR (400 MHz, MeOH-d₄): δ 8.60 (s, 1H), 7.95-7.86 (m, 3H), 6.15-6.87 (m, 8H), 6.01 (s, 2H), 4.52 (s, 2H), 4.14 (s, 2H), 3.43-3.30 (m, 2H), 2.92-2.85 (m, 2H), 2.81 (s, 2H), 2.37-2.34 (m, 2H), 1.77-1.70 (m, 2H); MS (ESI) m/z: Calculated for C₃₀H₂₈F₆N₂O₃: 578.2. found 579 (M+H)⁺.

### Example 33

### 1-(Benzo[d][1,3]dioxol-5-ylmethyl)-N-(3-fluoro-5-(trifluoromethyl)benzyl)-4-isobutylpiperidine-4-carboxamide

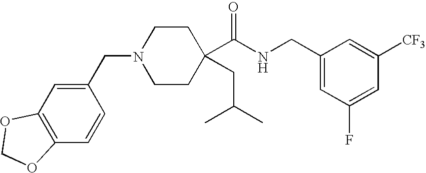

The title compound was prepared according to the general procedures described above in connection with Scheme 2 (47 mg, 31% yield): ¹H NMR (400 MHz, DMSO-d₆): δ 8.70 (br s, 1H), 7.60-7.40 (m, 3H), 7.20 (s, 1H), 7.05-6.95 (m, 2H), 6.05 (s, 2H), 4.40 (d, 2H), 4.15 (d, 2H), 3.25-3.00 (m, 2H), 2.80-2.65 (m, 2H), 2.40-2.20 (m, 2H), 1.80-1.60 (m, 2H), 1.50-1.35 (m, 3H), 0.70 (d, 6H); MS (ESI) m/z: Calculated for C₂₆H₃₀F₄N₂O₃: 494.2. found 495.2 (M+H)⁺.

### Example 34

### 1-(Benzo[d][1,3]dioxol-5-ylmethyl)-N-(3,5-dimethoxybenzyl)-4-isobutylpiperidine-4-carboxamide

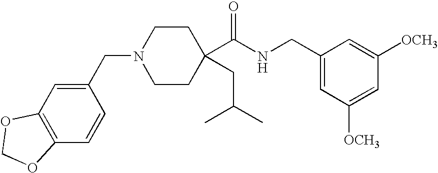

The title compound was prepared according to the general procedures described above in connection with Scheme 2 (30 mg, 20% yield): ¹H NMR (400 MHz, DMSO-d₆): δ 8.45 (br s, 1H), 7.25 (s, 1H), 7.00 (m, 2H), 6.50-6.35 (m, 3H), 6.05 (s, 2H), 4.25 (d, 2H), 4.15 (d, 2H), 3.40 (s, 6H), 3.25-3.00 (m, 2H), 2.80-2.70 (m, 2H), 2.40-2.30 (m, 2H), 1.80-1.60 (m, 2H), 1.55 (m, 1H), 1.40 (d, 2H), 0.80 (d, 6H); MS (ESI) m/z: Calculated for C₂₇H₃₆N₂O₅: 468.3. found 469.2 (M+H)⁺.

### Example 35

### 1-(4-(2H-tetrazol-5-yl)benzyl)-N-(3,5-bis(trifluoromethyl)benzyl)-4-isobutylpiperidine-4-carboxamide

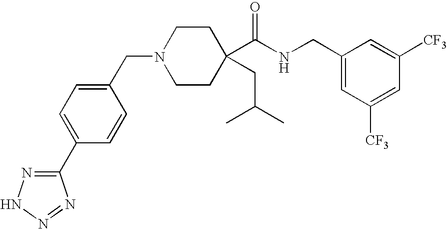

The title compound was prepared according to the general procedures described above in connection with Scheme 2 (25.0 mg, 6% yield): ¹H NMR (400 MHz, CDCl₃): δ 7.98-7.79 (m, 5H), 7.59 (d, 2H), 4.60 (d, 2H), 3.10-2.85 (m, 4H), 2.60-2.55 (m, 4H), 2.00-1.30 (m, 5H), 0.99 (d, 6H); Calculated for C₂₇H₃₀F₆N₆O: 568.2 found: 569.2 (M+H)⁺.

### Example 36

### 1-((4H-Imidazol-2-yl)methyl)-N-(3,5-bis(trifluoromethyl)benzyl)-4-isobutylpiperidine-4-carboxamide

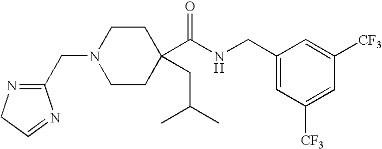

The title compound was prepared according to the general procedures described above in connection with Scheme 2 (3.0 mg TFA-salt, 2% yield): MS (ESI) m/z: Calculated for C₂₃H₂₈F₆N₄O: 490.2. found 491.3 (M+H)⁺.

### Example 37

### N-(3,5-Bis(trifluoromethyl)benzyl)-4-isobutyl-1-((6-methoxypyridin-3-yl)methyl)piperidine-4-carboxamide

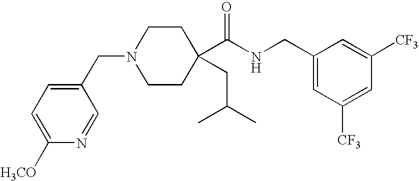

The title compound was prepared according to the general procedures described above in connection with Scheme 2 (3.0 mg TFA-salt, 2% yield): MS (ESI) m/z: Calculated for C₂₆H₃₁F₆N₃O₂: 531.2. found 532.3 (M+H)⁺.

### Example 38

### N-(3,5-Bis(trifluoromethyl)benzyl)-1-(3,4-dimethoxybenzyl)-4-isobutylpiperidine-4-carboxamide

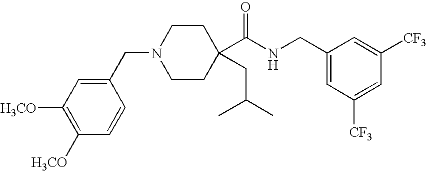

The title compound was prepared according to the general procedures described above in connection with Scheme 2 (2.1 mg TFA-salt, 1% yield): MS (ESI) m/z: Calculated for C₂₈H₃₄F₆N₂O₃: 560.3. found 561.1 (M+H)⁺.

### Example 39

### (1-(4-Hydroxy-3-methoxybenzyl)-4-isobutylpiperidin-4-yl)(4-(3-(trifluoromethyl)phenyl)piperazin-1-yl)methanone

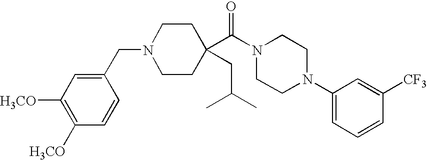

The title compound was prepared according to the general procedures described above in connection with Scheme 2 (2.8 mg TFA-salt, 1.5% yield): MS (ESI) m/z: Calculated for C₂₉H₃₈F₃N₃O₃: 533.3. found 534.1 (M+H)⁺.

### Example 40

### (1-(Benzo[d][1,3]dioxol-5-ylmethyl)-4-isobutylpiperidin-4-yl)(4-(3-(trifluoromethyl)phenyl)piperazin-1-yl)methanone

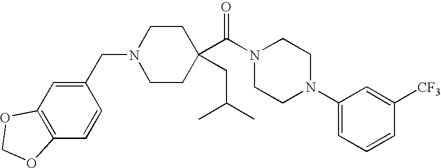

The title compound was prepared according to the general procedures described above in connection with Scheme 2 (4.5 mg TFA-salt, 2% yield): MS (ESI) m/z: Calculated for C₂₉H₃₆F₃N₃O₃: 531.3. found 532.2 (M+H)⁺.

### Example 41

### 1-(Benzo[d][1,3]dioxol-5-ylmethyl)-N-(3,5-bis(trifluoromethyl)benzyl)-4-(cyclopropylmethyl)piperidine-4-carboxamide

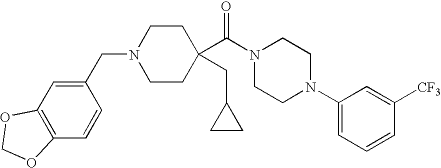

The title compound was prepared according to the general procedures described above in connection with Scheme 2 (19.0 mg TFA-salt, 12% yield): ¹H NMR (400 MHz, MeOH-d₄): δ 8.78 (m, 1H), 8.02 (s, 2H), 7.98 (s, 1H), 7.01 (m, 3H), 6.10 (s, 2H), 4.64 (d, 2H), 4.26 (s, 2H), 3.51 (m, 2H), 3.03 (m, 2H), 2.58 (m, 2H), 1.78 (m, 2H), 1.54 (d, 2H), 0.54 (m, 1H), 0.41 (m, 2H), 0.00 (m, 2H); MS (ESI) m/z: Calculated for C₂₇H₂₈F₆N₂O₃: 542.2. found 543.2 (M+H⁺).

### Example 42

### 1-(Benzo[d][1,3]dioxol-5-ylmethyl)-4-(3,5-bis(trifluoromethyl)benzyl)-N-isopropylpiperidine-4-carboxamide

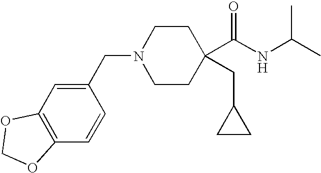

The title compound was prepared according to the general procedures described above in connection with Scheme 2 (7.2 mg, 99% yield): ¹H NMR (400 MHz, CDCl₃): δ 7.77 (s, 1H), 7.54 (d, 2H), 7.15 (s, 1H), 7.00 (m, 1H), 6.82 (d, 1H), 6.00 (s, 2H), 5.25 (bs, 1H), 3.98 (m, 3H), 3.48 (m, 2H), 2.99 (m, 2H), 2.65 (m, 2H), 2.55 (m, 2H), 2.16 (m, 2H), 1.10 (d, 6H); MS (ESI) m/z: Calculated for C₂₆H₂₈F₆N₂O₃: 530.2. found 531.2 (M+H⁺).

### Example 43

### 1-(Benzo[d][1,3]dioxol-5-ylmethyl)-N-(3,5-bis(trifluoromethyl)benzyl)-4-(isopropylsulfonyl)piperidine-4-carboxamide

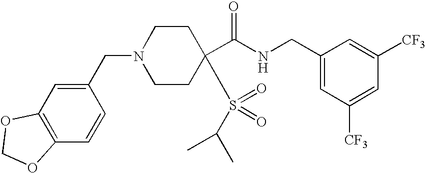

The title compound was prepared according to the general procedures described above in connection with Scheme 2 with an additional oxidation step described below for the appropriate intermediate (10.0 mg, 83% yield): ¹H NMR (400 MHz, MeOH-d₄): δ 9.15 (bs, 1H), 8.00-7.86 (m, 3H), 7.00-6.95 (m, 3H), 6.02 (s, 2H), 4.65 (d, 2H), 4.22 (s, 2H), 3.60-3.58 (m, 2H), 3.01-2.99 (m, 2H), 2.85-2.83 (m, 2H), 2.41-2.38 (m, 2H), 1.14 (d, 6H); MS (ESI) m/z: Calculated for C₂₆H₂₈F₆N₂O₅S: 594.2 found: 595.1 (M+H)⁺.

### 1-t-Butyl 4-ethyl 4-(isopropylsulfonyl)piperidine-1,4-dicarboxylate

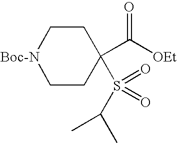

1-t-Butyl 4-ethyl 4-(isopropylthio)piperidine-1,4-dicarboxylate (1 g, 4.3 mmol) was dissolved in 20 mL of dry DCM. mCPBA (1.49 g, 8.7 mmol) was added and stirred for 2 h at RT. The reaction mixture was washed with a saturated NaHCO₃ solution and extracted with DCM. The extracted product was dried over Na₂SO₄ and purified by silica chromatography (1-3% dichloromethane/methanol) to give the desired product (0.82 g 75% yield): ¹H NMR (400 MHz, CDCl₃): δ 4.35 (q, 2H), 4.30-4.10 (m, 2H), 3.69-3.59 (m, 1H), 2.82-2.60 (m, 2H), 2.50-2.43 (m, 2H), 2.19-2.01 (m, 2H), 1.45 (s, 9H), 1.39 (d, 6H), 1.36 (t, 3H); MS (ESI) m/z: Calculated for C₁₆H₂₉NO₆S: 363.17 found: 264.1 (M−100+H)⁺.

### Example 44

### (R)-1-(Benzo[d][1,3]dioxol-5-ylmethyl)-N-(1-(3,5-bis(trifluoromethyl)phenyl)ethyl)-4-isobutylpiperidine-4-carboxamide

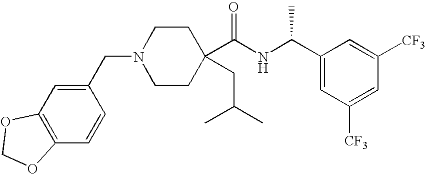

The title compound was prepared according to the general procedures described above in connection with Scheme 2 (10.3 mg, 14% yield): ¹H NMR (400 MHz, MeOH-d₄): δ 8.44 (d, 1H), 7.95 (s, 2H), 7.89 (s, 1H), 6.92 (m, 3H), 6.02 (s, 2H), 5.23 (m, 1H), 4.15 (s, 2H), 3.39 (m, 2H), 3.20 (m, 1H), 2.96 (t, 1H), 2.81 (t, 1H), 2.43 (m, 2H), 2.08 (m, 1H), 1.64 (m, 2H), 1.53 (m, 4H), 0.82 (d, 3H), 0.68 (d, 3H); MS (ESI) m/z: Calculated for C₂₈H₃₂F₆N₂O₃: 558.2 found: 559.3 (M+H)⁺.

### Example 45

### (S)-1-(benzo[d][1,3]dioxol-5-ylmethyl)-N-(1-(3,5-bis(trifluoromethyl)phenyl)ethyl)-4-isobutylpiperidine-4-carboxamide

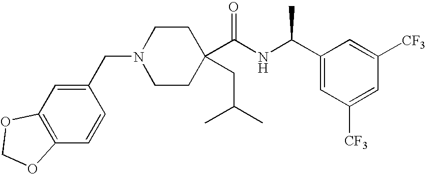

The title compound was prepared according to the general procedures described above in connection with Scheme 2 (8.7 mg, 8% yield): ¹H NMR (400 MHz, MeOH-d₄): δ 8.45 (d, 1H), 7.96 (s, 2H), 7.90 (s, 1H), 6.92 (m, 3H), 6.02 (s, 2H), 5.23 (m, 1H), 4.16 (s, 2H), 3.38 (m, 2H), 3.13 (m, 1H), 2.97 (t, 1H), 2.83 (t, 1H), 2.49 (m, 2H), 2.06 (m, 1H), 1.66 (m, 2H), 1.53 (m, 4H), 0.83 (d, 3H), 0.68 (d, 3H); MS (ESI) m/z: Calculated for C₂₈H₃₂F₆N₂O₃: 558.2 found: 559.5 (M+H)⁺.

### Example 46

### N-(3,5-Bis(trifluoromethyl)benzyl)-1-(2-fluoro-4,5-dimethoxybenzyl)-4-isobutylpiperidine-4-carboxamide

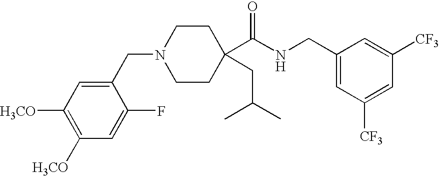

The title compound was prepared according to the general procedures described above in connection with Scheme 2 (5.0 mg TFA-salt, 4% yield): ¹H NMR (400 MHz, MeOH-d₄): δ 8.71 (m, 1H), 7.94 (s, 2H), 7.90 (s, 1H), 7.02 (d, 1H), 6.93 (d, 1H), 4.55 (d, 2H), 4.25 (s, 1H), 3.86 (s, 3H), 2.84 (s, 3H), 3.47 (m, 2H), 3.02 (m, 2H), 2.45 (m, 2H), 1.66 (m, 2H), 1.54 (m, 1H), 1.47 (d, 2H), 0.76 (d, 6H); MS (ESI) m/z: Calculated for C₂₈H₃₁F₇N₂O₃: 578.2. found 579.2 (M+H⁺).

### Example 47

### N-(3,5-Bis(trifluoromethyl)benzyl)-4-(cyclobutylmethyl)-1-(4-hydroxy-3-methoxybenzyl)piperidine-4-carboxamide

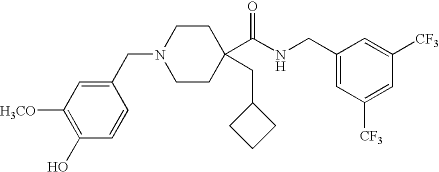

The title compound was prepared according to the general procedures described above in connection with Scheme 2 (21.0 mg TFA-salt, 17% yield): ¹H NMR (400 MHz, MeOH-d₄): δ 8.66 (m, 1H), 7.94 (s, 2H), 7.92 (s, 1H), 7.01 (s, 1H), 6.87 (m, 2H), 4.54 (s, 2H), 4.15 (s, 2H), 3.89 (s, 3H), 3.41 (m, 2H), 2.91 (m, 2H), 2.37 (m, 2H), 1.90 (m, 2H), 1.64 (m, 9H); MS (ESI) m/z: Calculated for C₂₈H₃₂F₆N₂O₃: 558.2. found 559.1 (M+H⁺).

### Example 48

### 1-(Benzo[d][1,3]dioxol-5-ylmethyl)-N-(3,5-bis(trifluoromethyl)benzyl)-4-(cyclobutylmethyl)piperidine-4-carboxamide

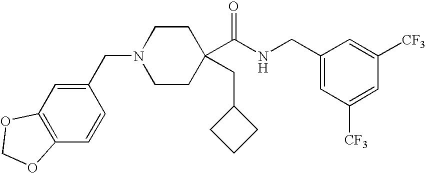

The title compound was prepared according to the general procedures described above in connection with Scheme 2 (15.0 mg TFA-salt, 12% yield): ¹H NMR (400 MHz, MeOH-d₄): δ 7.92 (m, 3H), 6.92 (m, 3H), 6.02 (s, 2H), 4.54 (s, 2H), 4.15 (s, 2H), 3.38 (m, 2H), 2.90 (m, 2H), 2.36 (m, 2H), 1.88 (m, 2H), 1.62 (m, 9H); MS (ESI) m/z: Calculated for C₂₈H₃₀F₆N₂O₃: 556.2. found 557.2 (M+H)⁺.

### Example 49

### N-(3,5-Bis(trifluoromethyl)benzyl)-1-(4-hydroxy-3-methoxybenzyl)-4-(oxazol-2-ylmethyl)piperidine-4-carboxamide

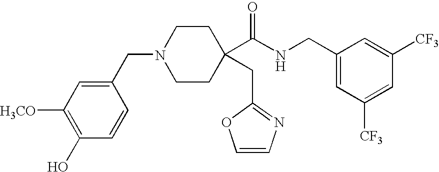

The title compound was prepared according to the general procedures described above in connection with Scheme 2 (10.0 mg, 88% yield): ¹H NMR (400 MHz, MeOH-d₄): δ 7.95-7.84 (m, 3H), 7.69 (s, 1H), 7.09 (s, 1H), 6.94 (s, 1H), 6.79-6.68 (m, 2H), 4.61 (s, 2H), 3.85 (s, 3H), 3.38-3.35 (m, 2H), 3.10 (s, 2H), 2.85-2.79 (m, 2H), 2.22-2.16 (m, 4H), 1.81-1.63 (m, 4H); MS (ESI) m/z: Calculated for C₂₇H₂₇F₆N₃O₄: 571.2. found: 572.0 (M+H)⁺.

### Example 50

### N-(3,5-Bis(trifluoromethyl)benzyl)-4-(cyclopropylmethyl)-1-((2,2-difluorobenzo[d][1,3]dioxol-5-yl)methyl)piperidine-4-carboxamide

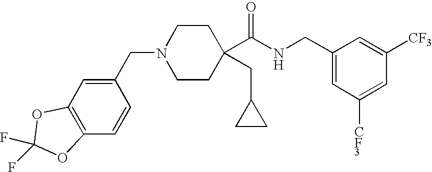

The title compound was prepared according to the general procedures described above in connection with Scheme 2 (4.7 mg TFA-salt, 4% yield): ¹H NMR (400 MHz, MeOH-d₄): δ 8.74 (m, 1H), 8.00 (s, 2H), 7.96 (s, 1H), 7.44 (s, 1H), 7.39 (m, 2H), 4.63 (d, 2H), 4.37 (s, 2H), 3.52 (m, 2H), 3.07 (m, 2H), 2.58 (m, 2H), 1.78 (m, 2H), 1.54 (d, 2H), 0.53 (m, 1H), 0.41 (m, 2H), 0.00 (m, 2H); Calculated for C₂₇H₂₆F₈N₂O₃: 578.2. found 579.2 (M+H)⁺.

### Example 51

### N-(3,5-Bis(trifluoromethyl)benzyl)-1-(1-(4-hydroxy-3-methoxyphenyl)ethyl)-4-isobutylpiperidine-4-carboxamide

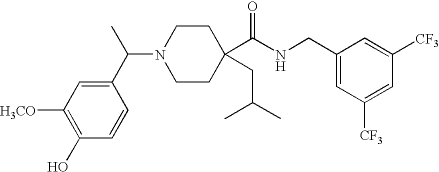

The title compound was prepared according to the general procedures described above in connection with Scheme 2 (1.7 mg TFA-salt, 0.6% yield): ¹H NMR (400 MHz, MeOH-d₄): δ 8.65 (m, 1H), 7.89 (s, 3H), 6.98 (s, 1H), 6.84 (s, 2H), 4.47 (d, 2H), 4.26 (d, 1H), 3.87 (s, 3H), 3.65 (m, 2H), 3.20 (m, 2H), 2.87 (m, 1H), 2.72 (m, 1H), 2.40 (m, 2H), 1.66 (d, 3H), 1.60 (m, 1H), 1.46 (m, 2H), 0.74 (s, 6H); MS (ESI) m/z: Calculated for C₂₈H₃₄F₆N₂O₃: 560.3. found 560.9 (M+H)⁺.

### Example 52

### 1-(Benzo[d][1,3]dioxol-5-ylmethyl)-N-(3,5-dimethylbenzyl)-4-isobutylpiperidine-4-carboxamide

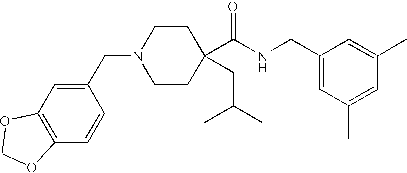

The title compound was prepared according to the general procedures described above in connection with Scheme 2 (22 mg, 18% yield): ¹H NMR (400 MHz, MeOH-d₄): δ 8.42 (bs, 1H), 7.00-6.82 (m, 6H), 6.01 (s, 2H), 4.30 (d, 2H), 4.18 (s, 2H), 3.43-3.28 (m, 2H), 2.94-2.82 (m, 2H), 2.45-2.38 (m, 2H), 2.25 (s, 6H), 1.78-1.40 (m, 5H), 0.82 (d, 6H); MS (ESI) m/z: Calculated for C₂₇H₃₆N₂O₃: 436.3. found 437.2 (M+H)⁺.

### Example 53

### N-(3,5-Bis(trifluoromethyl)benzyl)-4-isobutyl-1-(2-oxo-2-sulphonylmorpholinoethyl)piperidine-4-carboxamide

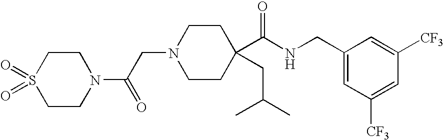

The title compound was prepared according to the general procedures described above in connection with Scheme 2 (8.7 mg, 31% yield): ¹H NMR (400 MHz, MeOH-d₄): δ 8.70 (br s 1H), 7.94 (2H), 7.89 (s, 1H), 4.54 (d, 2H), 4.29 (s, 2H), 4.08 (s, 2H), 3.83 (s, 2H), 3.57 (d, 2H), 3.19 (d, 2H), 3.08 (m, 1H), 2.43 (d, 2H), 2.22 (m, 1H), 2.01 (m, 1H), 1.82 (t, 2H), 1.60-1.52 (m, 4H), 0.79 (d, 6H); MS (ESI) m/z: Calculated for C₂₅H₃₃F₆N₃O₄S; 585.2. found 586.2 (M+H)⁺.

### Example 54

### N-(3,5-Bis(trifluoromethyl)benzyl)-4-isobutyl-1-(2-morpholino-2-oxoethyl)piperidine-4-carboxamide

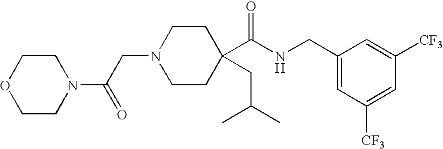

The title compound was prepared according to the general procedures described above in connection with Scheme 2 (13.8 mg, 41% yield): ¹H NMR (400 MHz, MeOH-d₄): δ 8.70 (br s 1H), 7.94 (2H), 7.88 (s, 1H), 4.54 (d, 2H), 4.19 (s, 2H), 3.67 (br s, 2H), 3.59-3.54 (m, 3H), 3.42-3.37 (m, 3H), 3.05 (t, 2H), 2.43 (d, 2H), 2.22 (m, 1H), 2.01 (m, 1H), 1.81 (t, 2H), 1.56-1.52 (m, 3H), 0.77 (d, 6H); MS (ESI) m/z: Calculated for C₂₅H₃₃F₆N₃O₃; 537.2. found 538.3 (M+H)⁺.

### Example 55

### N-(3,5-Bis(trifluoromethyl)benzyl)-4-((2,2-difluorocyclopropyl)methyl)-1-(4-hydroxy-3-methoxybenzyl)piperidine-4-carboxamide

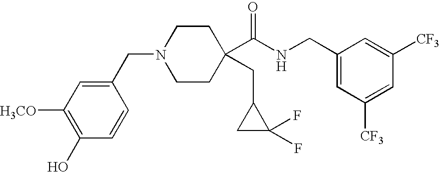

The title compound was prepared according to the general procedures described above in connection with Scheme 2 (71 mg TFA-salt, 37% yield): ¹H NMR (400 MHz, MeOH-d₄): δ 7.87 (m, 3H), 7.05 (m, 1H), 6.91 (m, 1H), 6.86 (m, 1H), 4.50 (s, 2H), 4.22 (s, 2H), 3.88 (s, 3H), 3.81 (m, 2H), 3.40 (m, 2H), 3.05 (m, 2H), 2.14 (m, 2H), 1.82 (d, 2H), 1.27 (m, 3H), 0.88 (m, 2H); MS (ESI) m/z: Calculated for C₂₇H₂₈F₈N₂O₃: 580.20. found 581.1 (M+H⁺).

### Example 56

### (4-Isobutyl-1-((tetrahydro-2H-pyran-4-yl)methyl)piperidin-4-yl)(4-(3-(trifluoromethyl)phenyl)piperazin-1-yl)methanone

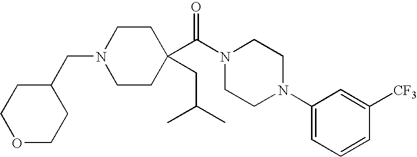

The title compound was prepared according to the general procedures described above in connection with Scheme 2 (91 mg TFA-salt, 33% yield): ¹H NMR (400 MHz, CDCl₃): δ 7.40 (dd, 1H), 7.17 (d, 1H), 7.12 (s, 1H), 7.08 (d, 1H), 3.96 (m, 2H), 3.82 (m, 4H), 3.59 (m, 2H), 3.71 (dd, 2H), 3.24 (m, 4H), 2.82 (m, 4H), 2.43 (m, 2H), 1.64 (m, 10H), 0.91 (d, 6H); MS (ESI) m/z: Calculated for C₂₇H₄₀F₃N₃O₂: 495.3. found 496.4 (M+H)⁺.

### Example 57

### N-(3,5-Bis(trifluoromethyl)benzyl)-1-(4-hydroxy-4-(6-methoxypyridin-3-yl)cyclohexyl)-4-isobutylpiperidine-4-carboxamide

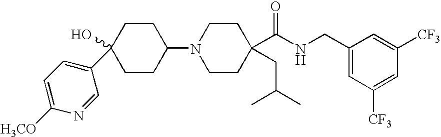

The title compound was prepared according to the general procedures described above in connection with Scheme 2. Procedure for purification and isolation of isomers is the same as example 26. Isomer A (7.1 mg, 3.4% yield) and Isomer B (10.0 mg, 4.6% yield): MS (ESI) m/z: Calculated for C₃₁H₃₉F₆N₃O₃: 615.2. found 616.2 (M+H)⁺ for Isomer A and 616.2 (M+H)⁺ for Isomer B.

### Example 58

### 5-((4-(3,5-Bis(trifluoromethyl)benzylcarbamoyl)-4-(cyclopropylmethyl)piperidin-1-yl)methyl)-2-fluorobenzoic acid

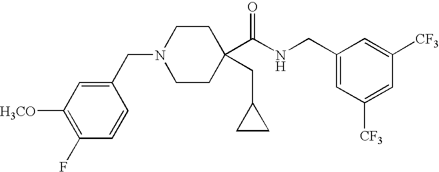

The title compound was prepared according to the general procedures described above in connection with Scheme 2 (50 mg TFA-salt, 31% yield): ¹H NMR (400 MHz, MeOH-d₄): δ 8.65 (m, 1H), 8.05 (dd, 1H), 7.93 (s, 2H), 7.89 (s, 1H), 7.66 (m, 1H), 7.31 (dd, 1H), 4.57 (d, 2H), 4.32 (s, 2H), 3.40 (m, 2H), 3.08 (m, 2H), 2.46 (m, 2H), 1.77 (m, 2H), 1.51 (m, 2H), 0.47 (m, 1H), 0.36 (m, 2H), −0.04 (m, 2H); MS (ESI) m/z: Calculated for C₂₇H₂₇F₇N₂O₃: 560.2. found 561.2 (M+H)⁺.

### Example 59

### N-(3,5-Bis(trifluoromethyl)benzyl)-4-(cyclopropylmethyl)-1-(4-fluoro-3-(2-(pyrrolidin-1-yl)ethylcarbamoyl)benzyl)piperidine-4-carboxamide

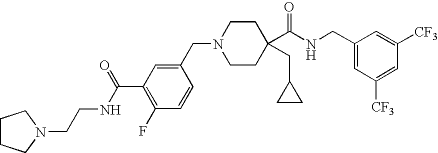

The title compound was prepared according to the general procedures described above in connection with Scheme 2 followed by an additional coupling step: 5-((4-(3,5-bis(trifluoromethyl)benzylcarbamoyl)-4-(cyclopropylmethyl)piperidin-1-yl)methyl)-2-fluorobenzoic acid (50.0 mg, 0.074 mmol), EDCI (28.0 mg, 0.15 mmol), HOBT (20 mg, 0.15 mmol) and 2-(pyrrolidin-1-yl)ethanamine were dissolved in DCM. Diisopropylethylamine (0.08 ml, 0.44 mmol) was added. The reaction was stirred over night at room temperature. The solution was extracted with saturated NaHCO₃. The organic solvent was evaporated. The crude product was purified by prep. HPLC (30-65% ACN (0.05 TFA) over 40 min) (1.1 mg TFA-salt, 2.1% yield): ¹H NMR (400 MHz, MeOH-d₄): δ 8.76 (m, 1H), 8.06 (dd, 1H), 8.00 (s, 2H), 7.95 (s, 1H), 7.75 (m, 1H), 7.43 (dd, 1H), 4.64 (d, 2H), 4.39 (s, 2H), 3.85 (dd, 4H), 3.52 (dd, 4H), 3.24 (m, 2H), 3.08 (m, 2H), 2.57 (m, 2H), 2.25 (m, 2H), 2.10 (m, 2H), 1.82 (m, 2H), 1.54 (d, 2H), 0.53 (m, 1H), 0.42 (m, 2H), 0.00 (m, 2H); MS (ESI) m/z: Calculated for C₃₃H₃₉F₇N₄O₂: 656.3. found 657.3 (M+H)⁺.

### Example 60

### N-(2-(4-(3,5-Bis(trifluoromethyl)benzylcarbamoyl)-4-isobutylpiperidin-1-yl)ethyl)-6-methoxynicotinamide

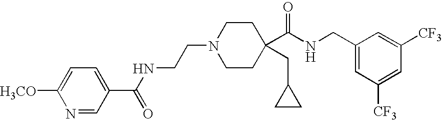

The title compound was prepared according to the general procedures described above in connection with Scheme 2 (80.0 mg, 30% yield): ¹H NMR (400 MHz, MeOH-d₄): δ

8.68-8.60 (m, 2H), 8.15 (d, 1H), 8.00-7.80 (m, 3H), 6.89 (d, 1H), 4.59 (d, 2H), 4.00 (s, 3H), 3.80-3.70 (m, 4H), 3.10-2.98 (m, 2H), 2.55-2.53 (m, 2H), 1.80-1.78 (m, 2H), 1.60-1.58 (m, 2H), 1.40-1.38 (d, 2H), 0.90-0.88 (m, 1H), 0.78 (d, 6H); Calculated for C₂₈H₃₄F₆N₄O₃: 588.3 found: 589.2 (M+H)⁺.

### Example 61

### 5-((4-(3,5-Bis(trifluoromethyl)benzylcarbamoyl)-4-(cyclopropylmethyl)piperidin-1-yl)methyl)-2-methoxybenzoic acid

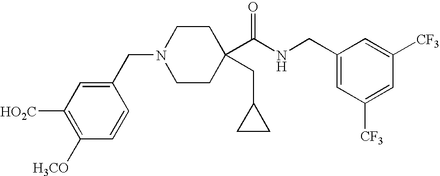

The title compound was prepared according to the general procedures described above in connection with Scheme 2 (2.1 mg TFA-salt, 0.6% yield). ¹H NMR (400 MHz, MeOH-d₄): δ 8.66 (m, 1H), 7.94 (m, 3H), 7.88 (s, 1H), 7.62 (d, 1H), 7.23 (d, 1H), 4.57 (s, 2H), 4.26 (s, 2H), 3.94 (s, 3H), 3.45 (m, 2H), 2.99 (m, 2H), 2.50 (m, 2H), 1.70 (m, 2H), 1.47 (d, 2H), 0.47 (m, 1H), 0.36 (m, 2H), 0.06 (m, 2H); MS (ESI) m/z: Calculated for C₂₈H₃₀F₆N₂O₄: 572.2. found 573.2 (M+H)⁺.

### Example 62

### 4-((4-(3,5-Bis(trifluoromethyl)benzylcarbamoyl)-4-(cyclopropylmethyl)piperidin-1-yl)methyl)-2-methoxybenzoic acid

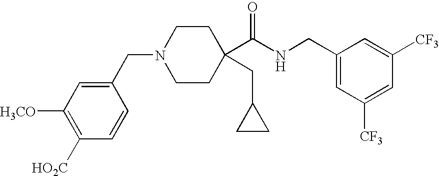

The title compound was prepared according to the general procedures described above in connection with Scheme 2 (8.6 mg, TFA-salt, 7% yield). ¹H NMR (400 MHz, MeOH-d₄): δ 8.79 (m, 1H), 8.01 (s, 2H), 7.96 (s, 1H), 7.93 (d, 1H), 7.34 (s, 1H), 7.18 (d, 1H), 4.65 (d, 2H), 4.39 (s, 2H), 4.00 (s, 3H), 3.54 (m, 2H), 3.10 (m, 2H), 2.58 (m, 2H), 1.84 (m, 2H), 1.55 (d, 2H), 0.53 (m, 1H), 0.41 (m, 2H), 0.00 (m, 2H); MS (ESI) m/z: Calculated for C₂₈H₃₀F₆N₂O₄: 572.2. found 573.2 (M+H)⁺.

### Example 63

### 5-((4-(3,5-Bis(trifluoromethyl)benzylcarbamoyl)-4-(cyclopropylmethyl)piperidin-1-yl)methyl)-2-hydroxybenzoic acid

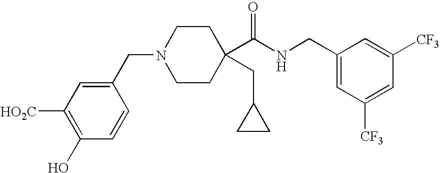

The title compound was prepared according to the general procedures described above in connection with Scheme 2 (37.0 mg, TFA-salt, 14% yield). ¹H NMR (400 MHz, MeOH-d₄): δ 8.77 (m, 1H), 8.09 (d, 1H), 8.01 (s, 2H), 7.96 (s, 1H), 7.66 (m, 1H), 7.08 (m, 1H), 4.64 (d, 2H), 4.38 (s, 2H), 3.57 (m, 2H), 3.10 (m, 2H), 2.57 (m, 2H), 1.81 (m, 2H), 1.54 (d, 2H), 0.55 (m, 1H), 0.43 (m, 2H), 0.00 (m, 2H); MS (ESI) m/z: Calculated for C₂₇H₂₈F₆N₂O₄: 558.2. found 559.2 (M+H)⁺.

### Example 64

### N-(3,5-Bis(trifluoromethyl)benzyl)-4-(cyclopropylmethyl)-1-(3-(ethylsulfonylcarbamoyl)-4-fluorobenzyl)piperidine-4-carboxamide

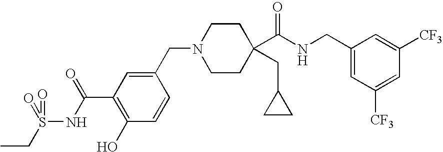

The title compound was prepared according to the same procedure as for example 63 using ethylsulfonamide instead of 2-(pyrrolidin-1-yl)ethanamine (2.8 mg, 10% yield). NMR (400 MHz, MeOH-d₄): δ 8.69 (m, 1H), 7.94 (s, 2H), 7.89 (s, 1H), 7.85 (m, 1H), 7.72 (m, 1H), 7.40 (m, 1H), 4.58 (d, 2H), 4.34 (s, 2H), 3.55 (q, 2H), 3.45 (m, 2H), 3.05 (m, 2H), 2.51 (m, 2H), 1.71 (m, 2H), 1.49 (m, 2H), 1.40 (t, 3H), 0.47 (m, 1H), 0.36 (m, 2H), −0.07 (m, 2H); MS (ESI) m/z: Calculated for C₂₉H₃₂F₇N₃O₄S: 651.2. found 652.2 (M+H)⁺.

### Example 65

### N-(3,5-Bis(trifluoromethyl)benzyl)-4-(cyclopropylmethyl)-1-(2-oxo-2-((tetrahydrofuran-2-yl)methylamino)ethyl)piperidine-4-carboxamide

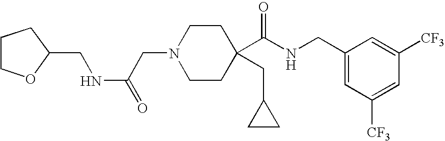

The title compound was prepared according to the general procedures described above in connection with Scheme 2 (48 mg, 36% yield). ¹H NMR (400 MHz, CDCl₃): δ 7.80 (s, 1H), 7.77 (s, 2H), 7.44 (t, 1H), 6.28 (t, 1H), 4.62 (d, 2H), 3.96 (m, 1H), 3.85 (m, 1H), 3.78 (m, 1H), 3.56 (m, 1H), 3.22 (m, 1H), 2.97 (s, 2H), 2.67 (d, 2H), 2.34 (t, 2H), 2.15 (m, 2H), 1.94 (m, 3H), 1.69 (m, 2H), 1.54 (m, 1H), 1.50 (d, 2H), 0.57 (m, 1H), 0.43 (dt, 2H), 0.00 (dt, 2H); MS (ESI) m/z: Calculated for C₂₆H₃₃F₆N₃O₃: 549.2. found: 550.2 (M+H)⁺.

### Example 66

### 4-((4-(3,5-Bis(trifluoromethyl)benzylcarbamoyl)-4-(cyclopropylmethyl)piperidin-1-yl)methyl)-2-fluorobenzoic acid

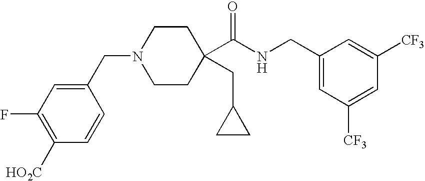

The title compound was prepared according to the general procedures described above in connection with Scheme 2 (20.1 mg, TFA-salt, 6% yield). ¹H NMR (400 MHz, MeOH-d₄): δ 8.72 (m, 1H), 8.07 (dd, 1H), 7.98 (s, 2H), 7.93 (s, 1H), 7.44 (d, 1H), 7.42 (s, 1H), 4.61 (d, 2H), 4.40 (s, 2H), 3.46 (m, 2H), 3.13 (m, 2H), 2.52 (m, 2H), 1.84 (m, 2H), 1.57 (m, 2H), 0.50 (m, 1H), 0.40 (m, 2H), 0.00 (m, 2H); MS (ESI) m/z: Calculated for C₂₇H₂₇F₇N₂O₃: 560.2. found 561.3 (M+H)⁺.

### Example 67

### N-(3,5-Bis(trifluoromethyl)benzyl)-1-((6-(4-cyanophenoxy)pyridin-3-yl)methyl)-4-(cyclopropylmethyl)piperidine-4-carboxamide

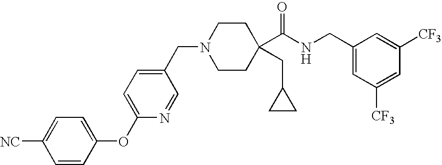

The title compound was prepared according to the general procedures described above in connection with Scheme 2 (74 mg, 50% yield). ¹H NMR (400 MHz, CDCl₃): δ 8.08 (d, 1H), 7.80 (s, 1H), 7.78 (s, 3H), 7.68 (d, 2H), 7.23 (d, 2H), 6.98 (d, 1H), 6.22 (bs, 1H), 4.62 (d, 2H), 3.43 (m, 2H), 2.69 (m, 2H), 2.17 (m, 4H), 1.68 (m, 2H), 1.48 (d, 2H), 0.57 (m, 1H), 0.42 (dt, 2H), 0.00 (dt, 2H); MS (ESI) m/z: Calculated for C₃₂H₃₀F₆N₄O₂: 616.2. found: 617.1 (M+H)⁺.

### Example 68

### 2-(2-((4-(3,5-Bis(trifluoromethyl)benzylcarbamoyl)-4-(cyclopropylmethyl)piperidin-1-yl)methyl)-6-methoxyphenoxy)acetic acid

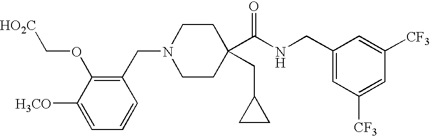

The title compound was prepared according to the general procedures described above in connection with Scheme 2 (50 mg, 35% yield). ¹H NMR (400 MHz, CDCl₃): δ 9.10 (t, 1H), 7.90 (s, 2H), 7.76 (s, 1H), 7.00 (s, 1H), 6.99 (s, 1H), 6.68 (t, 1H), 4.80 (s, 2H), 4.56 (d, 2H), 3.85 (s, 5H), 3.24 (d, 2H), 2.79 (t, 2H), 2.65 (d, 2H), 1.96 (t, 2H), 1.43 (d, 2H), 0.35 (m, 1H), 0.25 (dt, 2H), −0.27 (d, 2H); MS (ESI) m/z: Calculated for C₂₉H₃₂F₆N₂O₅: 602.2. found: 603.3 (M+H)⁺.

### Example 69

### N-(3,5-Bis(trifluoromethyl)benzyl)-4-(cyclopropylmethyl)-1-(2-methyl-2-morpholinopropyl)piperidine-4-carboxamide

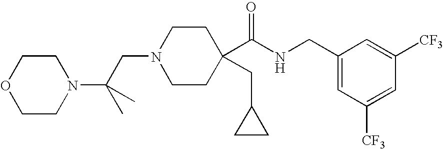

The title compound was prepared according to the general procedures described above in connection with Scheme 2 (12 mg, 9% yield). ¹H NMR (400 MHz, CDCl₃): δ 7.77 (s, 3H), 6.21 (s, 1H), 4.61 (d, 2H), 3.67 (t, 4H), 2.75 (m, 2H), 2.58 (t, 4H), 2.32 (t, 2H), 2.23 (s, 2H), 2.08 (d, 2H), 1.66 (m, 2H), 1.46 (d, 2H), 1.01 (s, 6H), 0.57 (m, 1H), 0.41 (dt, 2H), 0.00 (m, 2H); MS (ESI) m/z: Calculated for C₂₇H₃₇F₆N₃O₂: 549.3. found: 550.2 (M+H)⁺.

### Example 70

### N-(3,5-Bis(trifluoromethyl)benzyl)-4-(cyclopropylmethyl)-1-((2-morpholinothiazol-5-yl)methyl)piperidine-4-carboxamide

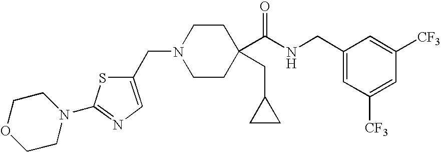

The title compound was prepared according to the general procedures described above in connection with Scheme 2 (34 mg, 24% yield). ¹H NMR (400 MHz, CDCl₃): δ 7.79 (s, 1H), 7.77 (s, 2H), 6.96 (s, 1H), 6.19 (t, 1H), 4.61 (d, 2H), 3.81 (t, 4H), 3.49 (s, 2H), 3.43 (t, 4H), 2.72 (d, 2H), 2.15 (m, 4H), 1.64 (m, 2H), 1.47 (d, 2H), 0.57 (m, 1H), 0.41 (dt, 2H), 0.02 (m, 2H); MS (ESI) m/z: Calculated for C₂₇H₃₂F₆N₄O₂S: 590.2. found: 590.8 (M+H)⁺.

### Example 71

### 1-(3-(1H-imidazol-1-yl)benzyl-N-(3,5-bis(trifluoromethyl)benzyl)-4-(cyclopropylmethyl)piperidine-4-carboxamide

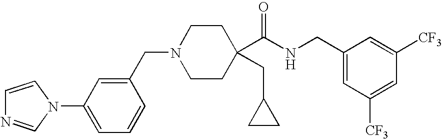

The title compound was prepared according to the general procedures described above in connection with Scheme 2 (35.0 mg, 13% yield): ¹H NMR (400 MHz, MeOH-d₄): δ 9.38 (d, 1H), 8.80 (bs, 1H), 8.11-7.75 (m, 9H), 4.64 (d, 2H), 4.46 (s, 2H), 3.60-3.45 (m, 2H), 3.20-3.00 (m, 2H), 2.60-2.53 (m, 2H), 1.90-1.79 (m, 2H), 1.54 (d, 2H), 0.50-0.01 (m, 5H); MS (ESI) m/z: Calculated for C₂₉H₃₀F₆N₄O: 564.2 found: 565.3 (M+H)⁺.

### Example 72

### N-(3,5-Bis(trifluoromethyl)benzyl)-1-((5-(4-chlorophenyl)isoxazol-3-yl)methyl)-4-(cyclopropylmethyl)piperidine-4-carboxamide

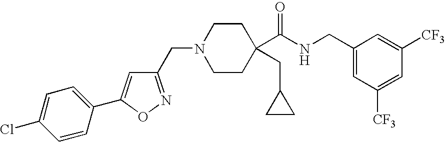

The title compound was prepared according to the general procedures described above in connection with Scheme 2 (34.0 mg TFA-salt, 25% yield): ¹H NMR (400 MHz, MeOH-d₄): δ 8.74 (m, 1H), 7.98 (s, 2H), 7.92 (m, 3H), 7.58 (d, 2H), 7.19 (s, 1H), 4.68 (s, 2H), 4.61 (d, 2H), 3.57 (m, 2H), 3.21 (m, 2H), 2.56 (m, 2H), 1.86 (m, 2H), 1.58 (m, 2H), 0.50 (m, 1H), 0.40 (m, 2H), 0.00 (m, 2H); MS (ESI) m/z: Calculated for C₂₉H₂₈ClF₆N₃O₂: 599.2. found 600.5 (M+H)⁺.

### Example 73

### N-(3,5-Bis(trifluoromethyl)benzyl)-4-(cyclopropylmethyl)-1-(3-((1,3-dimethyl-1H-pyrazol-5-yl)methoxy)-4-methoxybenzyl)piperidine-4-carboxamide

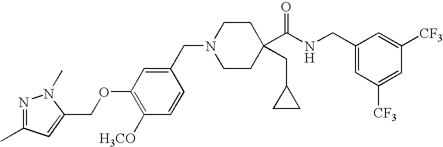

The title compound was prepared according to the general procedures described above in connection with Scheme 2 (6.8 mg, 23% yield): ¹H NMR: (400 MHz, MeOH-d₄): δ 8.78 (t, 1H, NH), 8.03 (s, 2H), 7.98 (m, 1H), 7.22 (s, 1H), 7.19-7.14 (m, 2H), 6.20 (s, 1H), 5.20 (s, 2H), 4.67 (d, 2H), 4.26 (s, 2H), 3.95 (s, 3H), 3.92 (s, 3H), 3.48 (d, 2H), 3.03 (t, 2H), 2.58 (d, 2H), 2.27 (d, 3H), 1.81 (m, 2H), 1.56 (d, 2H), 0.55-0.42 (m, 3H), 0.019 (m, 2H); MS (ESI) m/z: Calculated for C₃₃H₃₈F₆N₄O₃: 652.3. found 653.2 (M+H)⁺.

### Example 74

### N-(3,5-Bis(trifluoromethyl)benzyl)-4-(cyclopropylmethyl)-1-((6-(thiophen-2-yl)pyridin-3-yl)methyl)piperidine-4-carboxamide

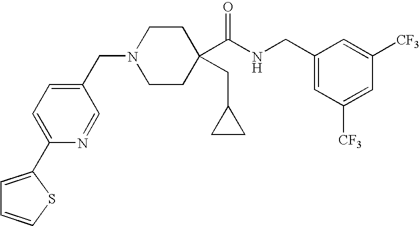

The title compound was prepared according to the general procedures described above in connection with Scheme 2 (7.9 mg TFA-salt, 5% yield): ¹H NMR (400 MHz, MeOH-d₄): δ 8.71 (m, 1H), 8.65 (s, 1H), 8.13 (m, 1H), 7.94 (m, 4H), 7.89 (m, 1H), 7.74 (m, 1H), 7.55 (dd, 1H), 4.58 (d, 2H), 4.37 (s, 2H), 3.51 (m, 2H), 3.08 (m, 2H), 2.54 (m, 2H), 1.72 (m, 2H), 1.48 (d, 2H), 0.47 (m, 1H), 0.34 (m, 2H), −0.06 (m, 2H); MS (ESI) m/z: Calculated for C₂₉H₂₉F₆N₃OS: 581.2. found 582.2 (M+H)⁺.

### Example 75

### N-(3,5-Bis(trifluoromethyl)benzyl)-4-(cyclopropylmethyl)-1-((1-(thiazol-2-yl)-1H-pyrrol-2-yl)methyl)piperidine-4-carboxamide

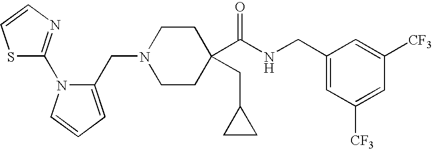

The title compound was prepared according to the general procedures described above in connection with Scheme 2 (27.0 mg TFA-salt, 21% yield): ¹H NMR (400 MHz, MeOH-d₄): δ 8.74 (m, 1H), 7.96 (s, 2H), 7.90 (s, 1H), 7.69 (d, 1H), 7.45 (d, 2H), 6.63 (m, 1H), 6.42 (m, 1H), 4.60 (d, 2H), 4.52 (s, 2H), 3.63 (d, 2H), 3.08 (m, 2H), 2.55 (d, 2H), 1.79 (d, 2H), 1.49 (d, 2H), 0.47 (m, 1H), 0.35 (m, 2H), −0.06 (m, 2H); MS (ESI) m/z: Calculated for C₂₇H₂₈F₆N₄OS: 570.2. found 571.2 (M+H)⁺.

### Example 76

### N-(3,5-Bis(trifluoromethyl)benzyl)-4-(cyclopropylmethyl)-1-((3-phenylisoxazol-5-yl)piperidine-4-carboxamide

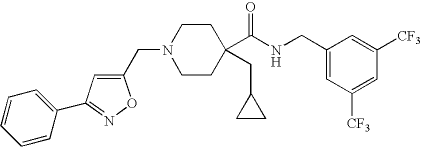

The title compound was prepared according to the general procedures described above in connection with Scheme 2 (52.0 mg, 20% yield): ¹H NMR (400 MHz, MeOH-d₄): δ 7.96-7.90 (m, 5H), 7.55-7.53 (m, 3H), 7.16 (s, 1H), 4.63 (s, 2H), 4.59 (s, 2H), 3.59-3.50 (m, 2H), 3.45-3.20 (m, 2H), 2.58-2.44 (m, 2H), 1.97-1.82 (m, 2H), 1.56 (d, 2H), 0.52-0.02 (m, 5H); MS (ESI) m/z: Calculated for C₂₉H₂₉F₆N₃O₂: 565.2. found: 566.2 (M+H)⁺.

### Example 77

### N-(3,5-Bis(trifluoromethyl)benzyl)-4-(cyclopropylmethyl)-1-(3-fluoro-4-(2-(pyrrolidin-1-yl)ethylcarbamoyl)benzyl)piperidine-4-carboxamide

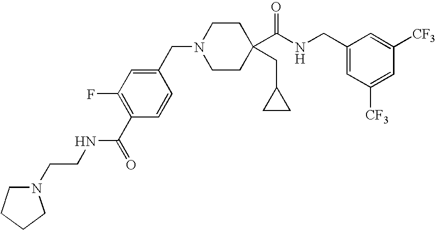

The title compound was prepared according to the same procedure as for example 59 (3.2 mg, 10% yield). ¹H NMR (400 MHz, MeOH-d₄): δ 8.75 (m, 1H), 7.99 (s, 3H), 7.95 (s, 1H), 7.48 (d, 2H), 4.63 (d, 2H), 4.42 (s, 2H), 3.84 (dd, 4H), 3.51 (dd, 4H), 3.22 (m, 2H), 3.09 (m, 2H), 2.55 (m, 2H), 2.24 (m, 2H), 2.09 (m, 2H), 1.82 (m, 2H), 1.55 (m, 2H), 0.51 (m, 1H), 0.41 (m, 2H), 0.00 (m, 2H); MS (ESI) m/z: Calculated for C₃₃H₃₉F₇N₄O₂: 656.3. found 657.2 (M+H)⁺.

### Example 78

### N-(3,5-Bis(trifluoromethyl)benzyl)-4-(cyclopropylmethyl)-1-(2,2-dimethyl-3-oxo-3-(thiazol-2-ylamino)propyl)piperidine-4-carboxamide

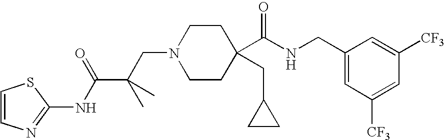

The title compound was prepared according to the general procedures described above in connection with Scheme 2 (1.1 mg, TFA-salt, 0.2% yield): ¹H NMR (400 MHz, MeOH-d₄): δ 8.60 (m, 1H), 7.93 (s, 2H), 7.87 (s, 1H), 7.19 (m, 1H), 6.85 (m, 1H), 4.55 (d, 2H), 3.53 (m, 2H), 2.97 (m, 1H), 2.39 (m, 2H), 1.73 (m, 1H), 1.52 (d, 2H), 1.49 (m, 2H), 1.36 (d, 2H), 1.28 (s, 6H), 0.49 (m, 1H), 0.37 (m, 2H), −0.04 (m, 2H); MS (ESI) m/z: Calculated for C₂₇H₃₂F₆N₄O₂S: 590.2. found 591.2 (M+H)⁺.

### Example 79

### 5-((3-(3,5-Bis(trifluoromethyl)benzylcarbamoyl)-3-(cyclopropylmethyl)pyrrolidin-1-yl)methyl)-2-fluorobenzoic acid

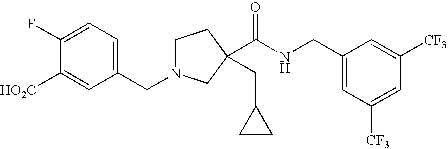

The title compound was prepared according to the general procedures described above in connection with Scheme 2 (2.8 mg, 4% yield)): ¹H NMR (400 MHz, MeOH-d₄): δ 8.8 (m, 1H), 8.16 (dd, 1H), 7.94 (s, 2H), 7.89 (s, 1H), 7.68 (m, 1H), 7.35 (dd, 1H), 4.51 (m, 4H), 4.08 (m, 1H), 3.47 (m, 1H), 2.48 (m, 1H), 2.21 (m, 1H), 2.00 (dd, 2H), 1.61 (m, 2H), 0.49 (m, 1H), 0.39 (m, 2H), 0.35 (m, 2H); MS (ESI) m/z: Calculated for C₂₆H₂₅F₇N₂O₃: 546.2. found 547.2 (M+H)⁺.

### Example 80

### N-(3,5-Bis(trifluoromethyl)benzyl)-3-(cyclopropylmethyl)-1-(3-fluoro-4-methoxybenzyl)pyrrolidine-3-carboxamide

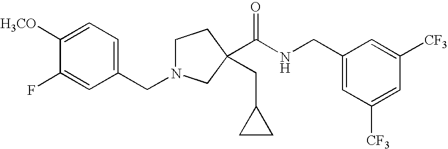

The title compound was prepared according to the general procedures described above in connection with Scheme 2 (1.7 mg, 2% yield): ¹H NMR (400 MHz, MeOH-d₄): δ 8.4 (m, 1H), 7.53 (m, 3H), 6.91 (m, 2H), 6.82 (m, 1H), 4.16 (s, 2H), 3.98 (m, 2H), 3.91 (m, 1H), 3.66 (m, 3.53 (s, 3H), 3.15 (m, 1H), 2.99 (m, 1H), 2.74 (d, 1H), 1.98 (m, 1H), 1.62 (m, 1H), 1.22 (m, 1H), 0.10 (m, 1H), 0.01 (m, 2H), −0.36 (m, 2H); MS (ESI) m/z: Calculated for C₂₆H₂₇F₇N₂O₂: 532.2. found 533.1 (M+H)⁺.

### Example 81

### N-(3,5-Bis(trifluoromethyl)benzyl)-3-(cyclopropylmethyl)-1-((tetrahydro-2H-pyran-4-yl)methyl)pyrrolidine-3-carboxamide

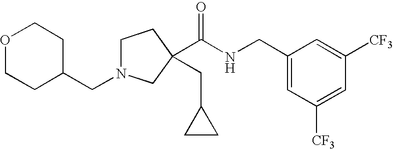

The title compound was prepared according to the general procedures described above in connection with Scheme 2 (21.0 mg, 56% yield): ¹H NMR (400 MHz, MeOH-d₄): δ 8.79 (m, 1H), 7.93 (s, 2H), 7.88 (s, 1H), 4.56 (m, 2H), 4.47 (d, 1H), 3.96 (m, 2H), 3.76 (m, 1H), 3.60 (m, 1H), 3.46 (m, 2H), 3.28 (m, 1H), 3.12-3.04 (m, 3H), 2.36-2.25 (m, 1H), 2.09 (m, 2H), 1.80-1.58 (m, 3H), 1.38 (m, 2H), 0.52 (m, 1H), 0.42 (m, 2H), 0.06 (d, 2H); MS (ESI) m/z: Calculated for C₂₄H₃₀F₆N₂O₂: 492.2. found 493.1 (M+H)⁺.

Activity data for selected invention compounds in the calcium flux assay is provided in the following Table

Activity Data

| Example | | IC₅₀^{a} |
|---|---|---|
| No | Structure | Ca⁺⁺ flux |
| 11 | 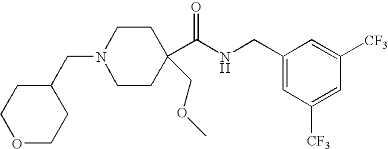 | + |
| 12 | 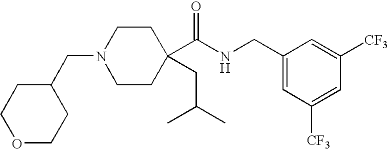 | + |
| 13 | 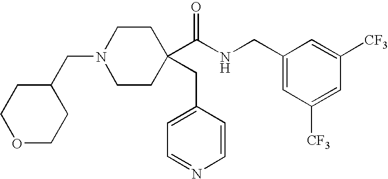 | + |
| 18 | 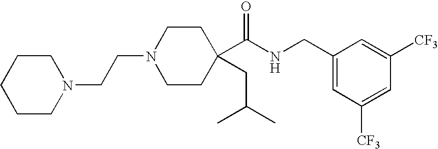 | + |
| 19 | 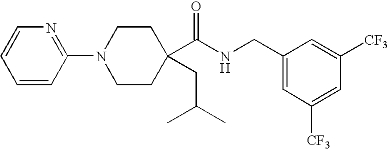 | + |
| 21 | 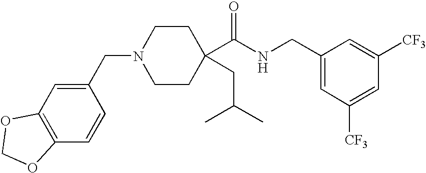 | ++ |
| 26 | 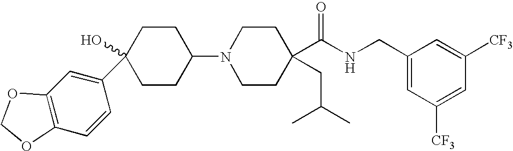 | ++ |
| 27 | 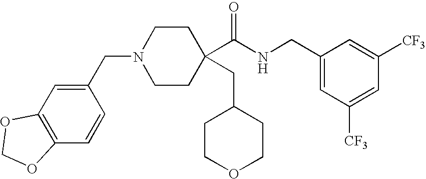 | + |
| 30 | 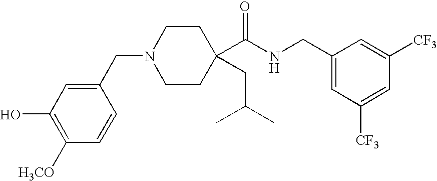 | +++ |
| 40 | 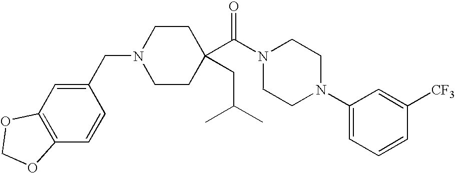 | + |
| 44 | 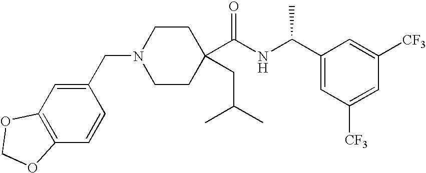 | + |
| 45 | 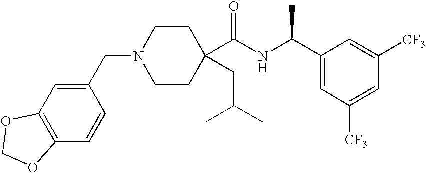 | + |
| 50 | 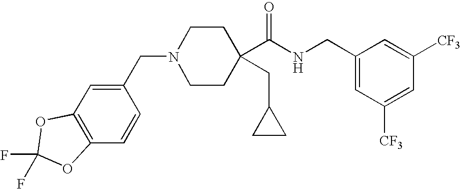 | +++ |
| 53 | 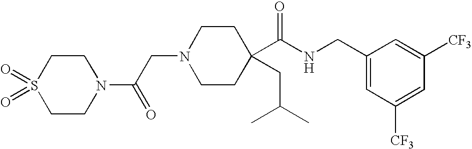 | + |
| 59 | 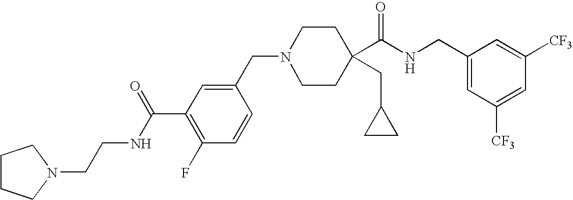 | + |
| 64 | 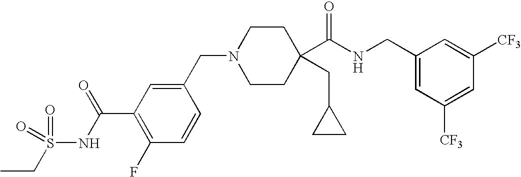 | ++ |
| 67 | 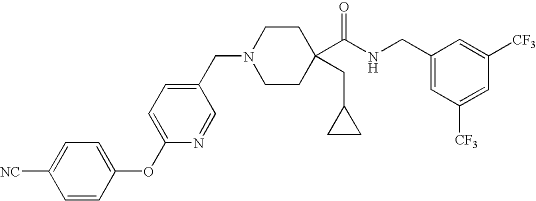 | ++ |
| 69 | 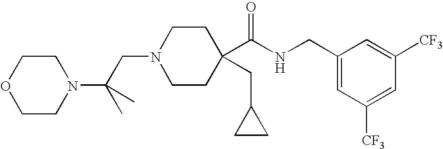 | ++ |
| 79 | 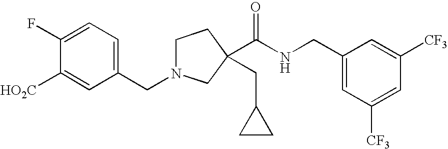 | ++ |
| 80 | 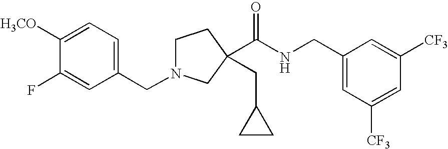 | ++ |
| 81 | 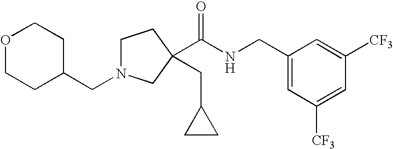 | +++ |

^{a} when IC₅₀ > 1000 nM: designates as “+”; when 1000 nM > IC₅₀ > 200 nM; designated as “++”; when IC₅₀ < 200 nM, designated as “+++”.

### EQUIVALENTS

Those skilled in the art will recognize, or be able to ascertain using no more than routine experimentation, numerous equivalents to the specific procedures described herein. Such equivalents are considered to be within the scope of the invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference for all purposes. The appropriate components, processes, and methods of those patents, applications and other documents may be selected for the invention and embodiments thereof.

1. A compound of Formula I-A: 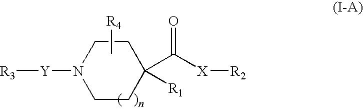
or a pharmaceutically acceptable salt thereof, wherein:
R₁ is hydrogen; alkyl, alkoxyalkyl, alkoxyphenyl, alkylthioalkyl, alkylamino, —SO₂(alkyl), —OR₈, —N(R₇)(R₈), C₃₋₆ cycloalkyl, C₃₋₆ heterocycloalkyl, aryl, heteroaryl, aralkyl, or heteroaralkyl, each of which is optionally substituted with 1, 2, or 3 occurrences of R₅; or R₁ is optionally substituted (C₁-C₆alkylene)-R₁ₐ, wherein R₁ₐ is C₃₋₆cycloalkyl, C₃₋₆heterocycloalkyl, aryl, or heteroaryl, each of which is optionally substituted with 1, 2, or 3 occurrences of R₅;
X is a direct bond, C(R₁₀)₂, NR₁₀, N(R₁₀)CO, N(R₁₀)SO₂, 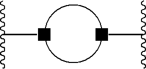
which is a 4, 5, or 6 membered ring wherein Z is N, CH, or C(C₁₋₃ alkyl); or X is NR₁₀(C₁-C₆alkylene), NR₁₀(C₁-C₆alkylene)SO₂—, or (C₁-C₆alkylene)SO₂—, wherein (C₁-C₆alkylene) is optionally substituted with 1, 2, or 3 substituents selected from the group consisting of halogen, methyl, and ethyl, or (C₁-C₆alkylene) is geminally substituted to form a cyclopropyl ring;
R₂ is cycloalkyl, aryl, heterocyclic, heteroaryl, aralkyl, heteroaralkyl, or aralkenyl; each of which is optionally substituted with 1, 2, or 3 occurrences of R₅;
Y is a direct bond, CO, SO₂, NHCO, NHSO₂, —C(═NR₁₀)—, C₁₋₄ alkylene, C₁₋₄ alkylene-SO₂—, —C(O)—C₁₋₄ alkylene, C₃₋₆ cycloalkylene, arylene, heterocycloalkylene, or heteroarylene; each of which is optionally substituted with 1, 2, or 3 occurrences of R₅;
R₃ is hydrogen or —N(alkyl)₂; or alkyl, alkenyl, alkynyl, cycloalkyl, aryl, heterocycloalkyl, or heteroaryl; each of which is optionally substituted with 1, 2, or 3 occurrences of R₅;
R₄ is hydrogen, C₁₋₈ alkyl, alkenyl, alkynyl, cycloalkyl, alkoxy, arylalkoxy, or heteroarylalkoxy;
R₅, when present, represents independently for each occurrence hydrogen, halogen, hydroxy, alkyl, alkenyl, cycloalkyl, alkoxy, —CO₂H, —CO₂C₁₋₃alkyl, —C(O)N(H)alkyl, —C(O)N(H)SO₂alkyl, —O-alkyleneCO₂R₁₀, cyano, oxo, aryl, heteroaryl, heterocyclic, alicyclic, CF₃, O—CF₃, O—CHF₂, —O-aryl, —N(alkyl)C(O)alkyl, —N(H)SO₂alkyl, C₁₋₃alkyl-S(O)₂—NH—, or C₁₋₃ alkyl-C(O)—NH—;
n is 0, 1 or 2;
R₇ is hydrogen or C₁₋₃ alkyl;
R₈ is alkyl, alicyclic, aryl, heterocyclic or heteroaryl; and
R₁₀ is hydrogen, C₁₋₂ alkyl, or C₁₋₂ alkenyl.

2. The compound of claim 1, wherein R₁ is hydrogen; alkyl, alkoxyalkyl, C₃₋₆ cycloalkyl, C₃₋₆ heterocycloalkyl, aryl, heteroaryl, or (C1-C6alkylene)-R1a, wherein R1a is C3-6 cycloalkyl, C3-6 heterocycloalkyl, aryl, or heteroaryl, each of which is optionally substituted with 1, 2, or 3 occurrences of R₅.

3. The compound of claim 1, wherein R₁ 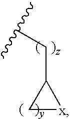
wherein
z is 1, 2, or 3;
y is 1, 2, 3, or 4; and
x is O, NH, CH₂, CF₂, or N—C₁₋₈ alkyl.

4. The compound of claim 1, wherein R₁ is methyl; 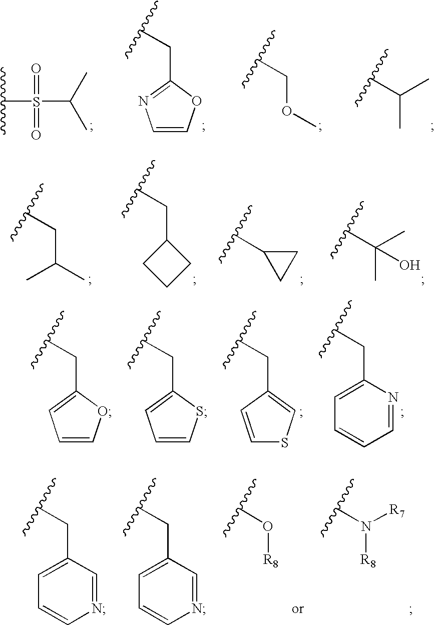
any of which is optionally substituted on carbon with 1, 2, or 3 fluorine atoms, and wherein:
R₇ is hydrogen or C₁₋₃ alkyl; and
R₈ is alkyl, alicyclic, aryl, heterocyclic, or heteroaryl.

5. The compound of claim 1, wherein R₂ is cycloalkyl, aryl, heterocycloalkyl, or heteroaryl; each of which is optionally substituted with 1, 2, or 3 occurrences of R₅.

6. The compound of claim 1, wherein R₂ is

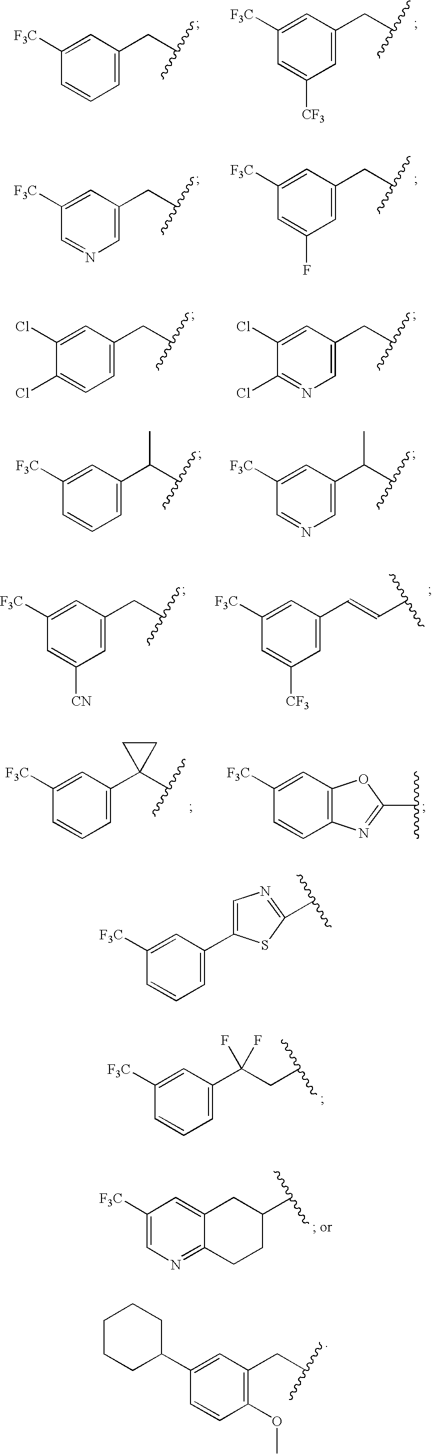

7. The compound of claim 1, wherein X is NH; N-methyl; N-ethyl; NHCH₂; N(methyl)-CH₂; N(ethyl)-CH₂; CH₂; CH(methyl); CH(ethyl); NHCO; NHSO₂; or a direct bond.

8. The compound of claim 1, wherein X—R₂ is

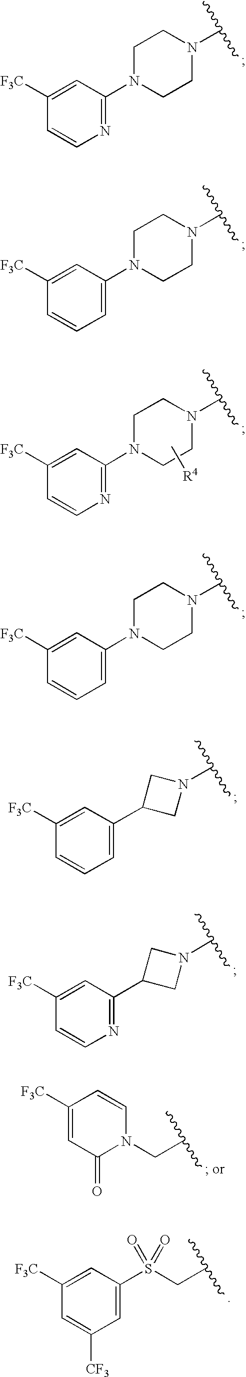

9. The compound of claim 1, wherein Y is 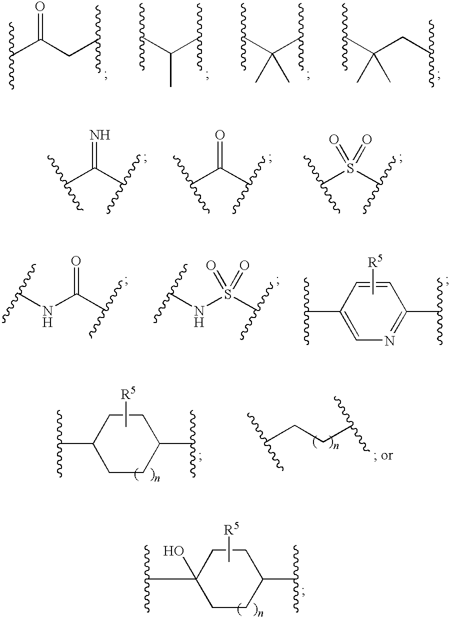
wherein n is 0, 1 or 2.

10. The compound of claim 1, wherein R₃ is 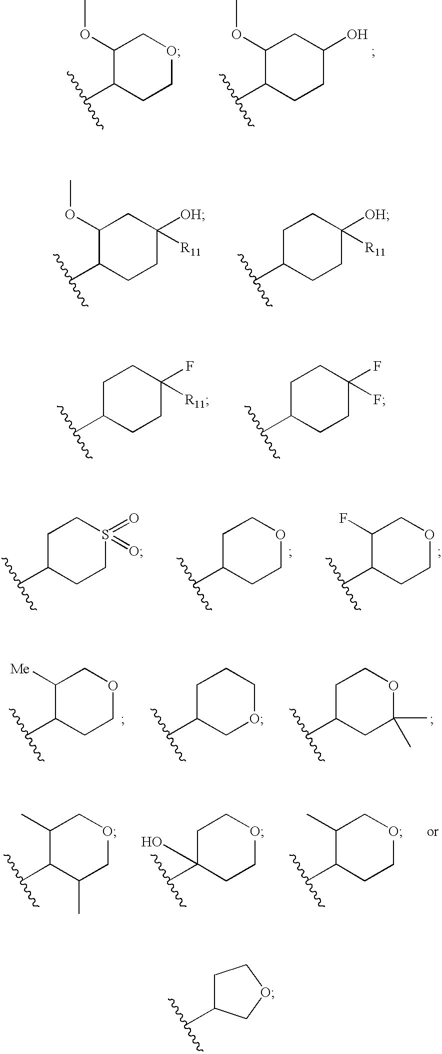
wherein R₁₁ is selected from the group consisting of an substituted or unsubstituted alicyclic, aryl, heterocyclic, or heteroaryl.

11. The compound of claim 1, wherein R₃ is 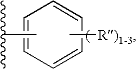
wherein R″ represents independently for each occurrence hydroxyl, halo, alkoxy, halo-alkoxy, C₁₋₃ alkyl-S(O)₂—NH—, —CO₂H, C₁₋₃ alkyl-C(O)—NH—, aryl or halo-substituted aryl, or hetereoaryl; or wherein there are two R″ attached to adjacent carbons, the two R″ groups taken together form

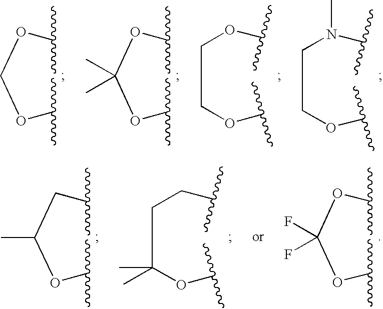

12. The compound of claim 1, wherein R₃ is 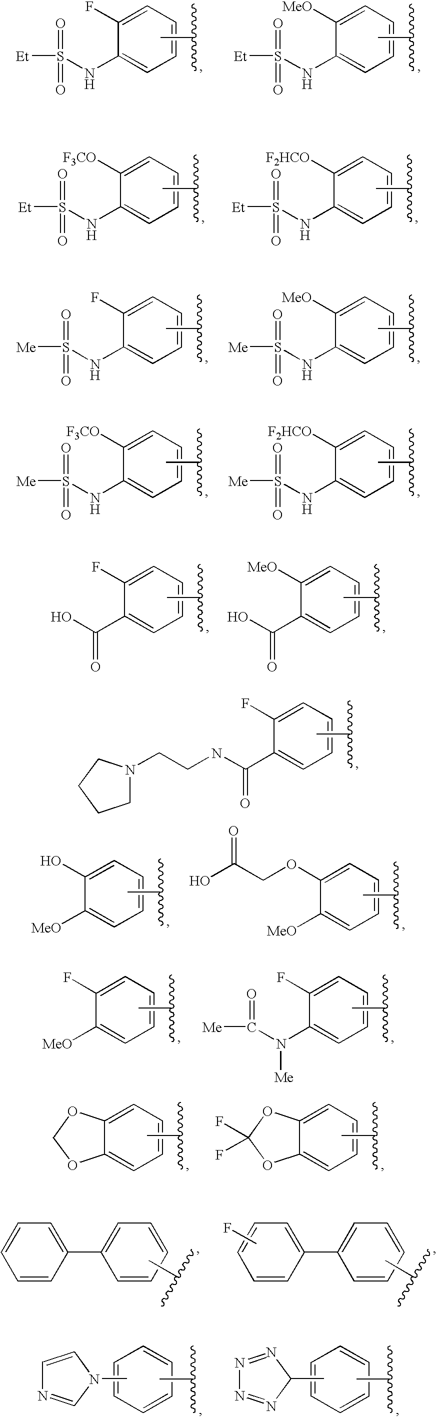 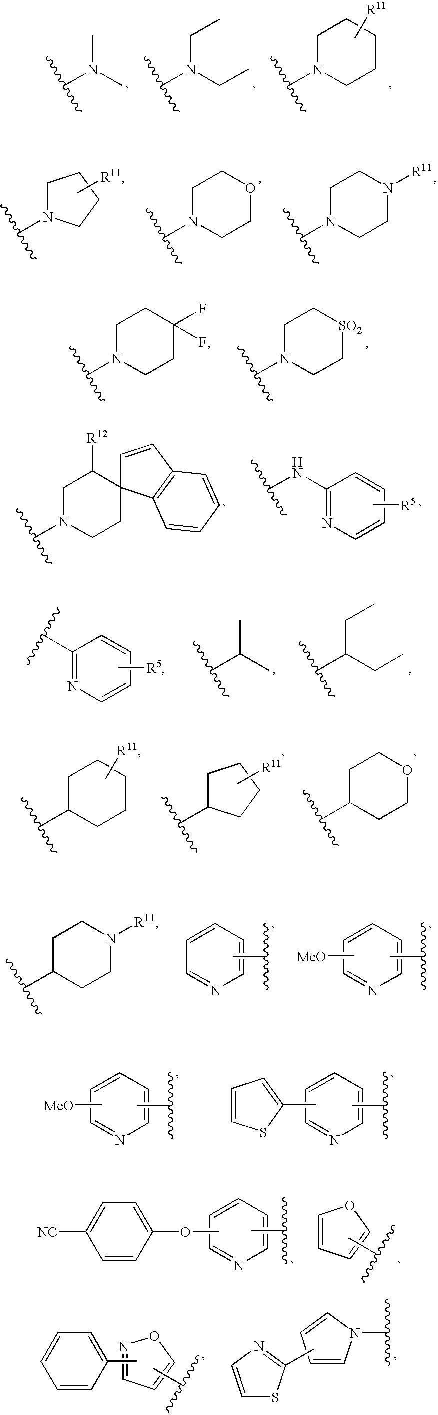 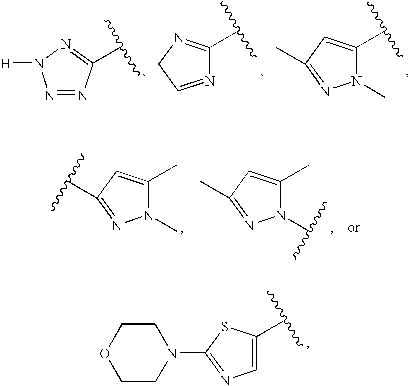
wherein R¹¹ is hydrogen, lower alkyl, hydroxyl, amino, alkoxy, or SO₂-lower alkyl; or an unsubstituted alicyclic, aryl, heterocyclic or heteroaryl; and R¹² is hydrogen or C₁₋₃ alkyl.

13. The compound of claim 1, which is a compound of formula II 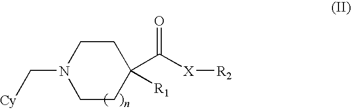
or a pharmaceutically acceptable salt thereof, wherein:
R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, or heteroaryl ring, each of which is optionally substituted with 1, 2, or 3 occurrences of R₅; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅;
X is a direct bond, NR₁₀, NR₁₀CO, NR₁₀SO₂, 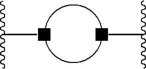
which is a 4, 5, or 6 membered ring wherein Z is N, CH, or C(C₁₋₃ alkyl); or NR₁₀(C₁-C₆alkylene), NR₁₀(C₁-C₆alkylene)SO₂—, wherein (C₁-C₆alkylene) is optionally substituted with 1, 2, or 3 halo, methyl, or ethyl groups, or is geminally substituted to form a cyclopropyl ring;
R₂ is cycloalkyl, aryl, heterocycloalkyl, heteroaryl, aralkyl, heteroaralkyl; each of which is optionally substituted with 1, 2, or 3 occurrences of R₅;
R₅, when present, is hydrogen, halogen, hydroxy, lower alkyl, lower alkenyl, cycloalkyl, C₁₋₃ alkoxy, —CO₂H, —CO₂C₁₋₃alkyl, cyano, aryl, heteroaryl, oxo, CF₃, O—CF₃, or O—CHF₂;
n is 0, 1 or 2;
R₁₀ is hydrogen, C₁₋₂ alkyl, or C₁₋₂ alkenyl; and
Cy is an unsubstituted cyclic or bicyclic ring optionally having partial aromaticity and optionally having one or more heteroatoms.

14. The compound of claim 1, which is a compound of formula III 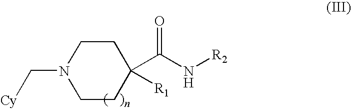
or a pharmaceutically acceptable salt thereof, wherein:
R₁ is hydrogen; alkyl, alkoxyalkyl, alkoxyphenyl, alkylthioalkyl, alkylamino, —SO₂(alkyl), C₃₋₆ cycloalkyl, C₃₋₆ heterocycloalkyl, aryl, or heteroaryl ring, each of which is optionally substituted with 1, 2, or 3 occurrences of R₅; or R₁ is optionally substituted (C₁-C₆alkylene)-R₁ₐ, wherein R₁ₐ is C₃₋₆ cycloalkyl, C₃₋₆ heterocycloalkyl, aryl, or heteroaryl, each of which is optionally substituted with 1, 2, or 3 occurrences of R₅;
R₂ is cycloalkyl, aryl, heterocycloalkyl, heteroaryl, aralkyl, or heteroaralkyl; each of which is optionally substituted with 1, 2, or 3 occurrences of R₅;
R₅, when present, is hydrogen, halogen, hydroxy, lower alkyl, lower alkenyl, cycloalkyl, C₁₋₃ alkoxy, —CO₂H, —CO₂C₁₋₃alkyl, cyano, aryl, heteroaryl, oxo, CF₃, O—CF₃, or O—CHF₂;
n is 0, 1 or 2;
R₁₀ is hydrogen, C₁₋₂ alkyl, or C₁₋₂ alkenyl; and
Cy is an unsubstituted cyclic or bicyclic ring optionally having partial aromaticity and optionally having one or more heteroatoms.

15. The compound of claim 1, which is a compound of Formula IV-A:
wherein: 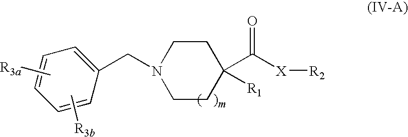
m is 0 or 1; and
R₃ₐ and R_{3b} are each independently hydrogen, halo, hydroxy, lower alkyl, lower alkenyl, cycloalkyl, C₁₋₃ alkoxy, cyano, or CF₃, or R₃ₐ and R_{3b} taken together form

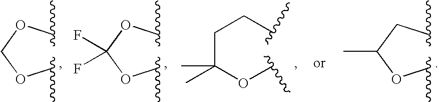

16. The compound of claim 15, which is a compound of Formula IV-B:

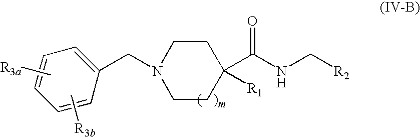

17. The compound of claim 16, which is a compound of Formula IV-C: 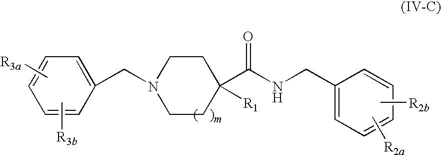
wherein R₂ₐ and R_{2b} are each independently hydrogen, halo, hydroxy, alkyl, haloalkyl, alkoxy, haloalkoxy, cyano, or CF₃.

18. The compound of claim 17, which is a compound of Formula IV-D. 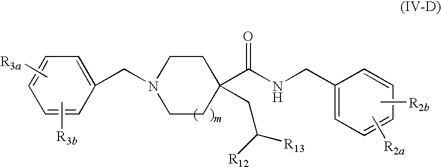
wherein R₁₂ and R₁₃ are each independently optionally substituted alkyl or taken together with the carbons to which they are attached form a 3, 4, 5, or 6-membered ring optionally containing one heteroatom selected from O, S, NH, or N-alkyl, which ring is optionally substituted with 1, 2, or 3 groups selected from the group consisting of halo, alkyl, alkoxy, and haloalkoxy.

19. The compound of claim 18, which is a compound of Formula IV-E.

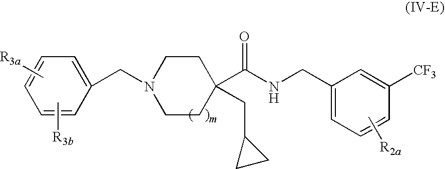

20. The compound of claim 1 which is:
N-(3,5-Bis(trifluoromethyl)benzyl)-1-((tetrahydro-2H-pyran-4-yl)methyl)-4-methylpiperidine-4-carboxamide;
N-(3,5-Bis(trifluoromethyl)benzyl)-1-((benzo[d][1,3]dioxol-5-yl)methyl)-4-methylpiperidine-4-carboxamide;
1-(Benzo[d][1,3]dioxol-5-ylmethyl)-N-(3,4-dichlorobenzyl)-4-methylpiperidine-4-carboxamide;
N-(3,4-dichlorobenzyl)-4-methyl-1-(tetrahydro-2H-pyran-4-yl)methyl)piperidine-4-carboxamide;
1-(Benzo[c][1,2,5]oxadiazol-5-ylmethyl)-N-(3,4-dichlorobenzyl)-4-methylpiperidine-4-carboxamide;
1-(Benzo[c][1,2,5]oxadiazol-5-ylmethyl)-N-(3,4-dichlorobenzyl)-4-methylpiperidine-4-carboxamide;
1-(Benzo[c][1,2,5]oxadiazol-5-ylmethyl)-N-(3,4-dichlorobenzyl)-4-methylpiperidine-4-carboxamide;
N-(3-Fluoro-5-(trifluoromethyl)benzyl)-4-methyl-1-((tetrahydro-2H-pyran-4-yl)methyl)piperidine-4-carboxamide;
N-(3,5-Bis(trifluoromethyl)benzyl)-1-((tetrahydro-2H-pyran-4-yl)methyl)-4-(methoxymethyl)piperidine-4-carboxamide;
N-(3,5-Bis(trifluoromethyl)benzyl)-1-((tetrahydro-2H-pyran-4-yl)methyl)-4-isobutylpiperidine-4-carboxamide;
N-(3,5-Bis(trifluoromethyl)benzyl)-4-(pyridin-4-ylmethyl)-1-((tetrahydro-2H-pyran-4-yl)methyl)piperidine-4-carboxamide;
4-Benzyl-N-(3,5-bis(trifluoromethyl)benzyl)-1-((tetrahydro-2H-pyran-4-yl)methyl)piperidine-4-carboxamide;
N-(3,5-Bis(trifluoromethyl)benzyl)-4-(4-methoxybenzyl)-1-((tetrahydro-2H-pyran-4-yl)methyl)piperidine-4-carboxamide;
N-(3,5-Bis(trifluoromethyl)benzyl)-1-(tetrahydro-2H-pyran-4-yl)methyl)-4-(thiophene-3-ylmethyl)piperidine-4-carboxamide;
N-(3,5-Bis(trifluoromethyl)benzyl)-4-(pyridin-3-ylmethyl)-1-((tetrahydro-2H-pyran-4-yl)methyl)piperidine-4-carboxamide;
N-(3,5-Bis(trifluoromethyl)benzyl)-4-isobutyl-1-(2-(piperidin-1-yl)ethyl)piperidine-4-carboxamide;
N-(3,5-Bis(trifluoromethyl)benzyl)-4-isobutyl-1-(pyridin-2-yl)piperidine-4-carboxamide;
N-(3,5-Bis(trifluoromethyl)benzyl)-1-(furan-2-ylmethyl)-4-isobutylpiperidine-4-carboxamide;
1-(Benzo[d][1,3]dioxol-5-ylmethyl)-N-(3,5-bis(trifluoromethyl)benzyl)-4-isobutylpiperidine-4-carboxamide;
1-(Benzo[d][1,3]dioxol-5-ylmethyl)-N-(3,5-bis(trifluoromethyl)benzyl)-4-(pyridin-3-ylmethyl)piperidine-4-carboxamide;
1-(Benzo[d][1,3]dioxo-5-ylmethyl)-N-(3,5-bis(trifluoromethyl)benzyl)-4-(thiophene-3-ylmethyl)piperidine-4-carboxamide;
1-(Benzo[d][1,3]dioxol-5-ylmethyl)-N-(3,5-bis(trifluoromethyl)benzyl)-4-(pyridin-4-ylmethyl)piperidine-4-carboxamide;
1-(Benzo[d][1,3]dioxol-5-ylmethyl)-4-isobutyl-N-(3-(trifluoromethyl)benzyl)piperidine-4-carboxamide;
1-(4-(Benzo[d][1,3]dioxol-5-yl)-4-hydroxycyclohexyl)-N-(3,5-bis(trifluoromethyl)benzyl)-4-isobutylpiperidine-4-carboxamide
1-(4-(Benzo[d][1,3]dioxol-5-yl)-4-hydroxycyclohexyl)-N-(3,5-bis(trifluoromethyl)benzyl)-4-isobutylpiperidine-4-carboxamide;
1-(Benzo[d][1,3]dioxol-5-ylmethyl)-N-(3,5-bis(trifluoromethyl)benzyl)-4-((tetrahydro-2H-pyran-4-yl)methyl)piperidine-4-carboxamide;
1-(Benzo[d][1,3]dioxol-5-ylmethyl)-N-(3,5-bis(trifluoromethyl)benzyl)-4-(4-methoxybenzyl)piperidine-4-carboxamide;
1-(Benzo[d][1,3]dioxol-4-ylmethyl)-N-(3,5-bis(trifluoromethyl)benzyl)-4-isobutylpiperidine-4-carboxamide;
N-(3,5-Bis(trifluoromethyl)benzyl)-1-(3-hydroxy-4-methoxybenzyl)-4-isobutylpiperidine-4-carboxamide;
N-(3,5-Bis(trifluoromethyl)benzyl)-1-(4-hydroxy-3-methoxybenzyl)-4-isobutylpiperidine-4-carboxamide;
1-(Benzo[d][1,3]dioxol-5-ylmethyl)-4-benzyl-N-(3,5-bis(trifluoromethyl)benzyl)piperidine-4-carboxamide;
1-(Benzo[d][1,3]dioxol-5-ylmethyl)-N-(3-fluoro-5-(trifluoromethyl)benzyl)-4-isobutylpiperidine-4-carboxamide;
1-(Benzo[d][1,3]dioxol-5-ylmethyl)-N-(3,5-dimethoxybenzyl)-4-isobutylpiperidine-4-carboxamide;
1-(4-(2H-tetrazol-5-yl)benzyl)-N-(3,5-bis(trifluoromethyl)benzyl)-4-isobutylpiperidine-4-carboxamide;
1-((4H-Imidazol-2-yl)methyl)-N-(3,5-bis(trifluoromethyl)benzyl)-4-isobutylpiperidine-4-carboxamide;
N-(3,5-Bis(trifluoromethyl)benzyl)-4-isobutyl-1-((6-methoxypyridin-3-yl)methyl)piperidine-4-carboxamide;
N-(3,5-Bis(trifluoromethyl)benzyl)-1-(3,4-dimethoxybenzyl)-4-isobutylpiperidine-4-carboxamide;
(1-(4-Hydroxy-3-methoxybenzyl)-4-isobutylpiperidin-4-yl)(4-(3-(trifluoromethyl)phenyl)piperazin-1-yl)methanone;
(1-(Benzo[d][1,3]dioxol-5-ylmethyl)-4-isobutylpiperidin-4-yl)(4-(3-(trifluoromethyl)phenyl)piperazin-1-yl)methanone;
N-(3,5-Bis(trifluoromethyl)benzyl)-4-(cyclopropylmethyl)-1-(4-hydroxy-3-methoxybenzyl)piperidine-4-carboxamide;
1-(Benzo[d][1,3]dioxol-5-ylmethyl)-N-(3,5-bis(trifluoromethyl)benzyl)-4-(cyclopropylmethyl)piperidine-4-carboxamide;
1-(Benzo[d][1,3]dioxol-5-ylmethyl)-4-(3,5-bis(trifluoromethyl)benzyl)-N-isopropylpiperidine-4-carboxamide;
1-(Benzo[d][1,3]dioxol-5-ylmethyl)-N-(3,5-bis(trifluoromethyl)benzyl)-4-(isopropylsulfonyl)piperidine-4-carboxamide;
(R)-1-(Benzo[d][1,3]dioxol-5-ylmethyl)-N-(1-(3,5-bis(trifluoromethyl)phenyl)ethyl)-4-isobutylpiperidine-4-carboxamide;
(S)-1-(benzo[d][1,3]dioxol-5-ylmethyl)-N-(1-(3,5-bis(trifluoromethyl)phenyl)ethyl)-4-isobutylpiperidine-4-carboxamide;
N-(3,5-Bis(trifluoromethyl)benzyl)-1-(2-fluoro-4,5-dimethoxybenzyl)-4-isobutylpiperidine-4-carboxamide;
N-(3,5-Bis(trifluoromethyl)benzyl)-4-(cyclobutylmethyl)-1-(4-hydroxy-3-methoxybenzyl)piperidine-4-carboxamide;
1-(Benzo[d][1,3]dioxol-5-ylmethyl)-N-(3,5-bis(trifluoromethyl)benzyl)-4-(cyclobutylmethyl)piperidine-4-carboxamide;
N-(3,5-Bis(trifluoromethyl)benzyl)-1-(4-hydroxy-3-methoxybenzyl)-4-(oxazol-2-ylmethyl)piperidine-4-carboxamide;
N-(3,5-Bis(trifluoromethyl)benzyl)-4-(cyclopropylmethyl)-1-((2,2-difluorobenzo[d][1,3]dioxol-5-yl)methyl)piperidine-4-carboxamide;
N-(3,5-Bis(trifluoromethyl)benzyl)-1-(1-(4-hydroxy-3-methoxyphenyl)ethyl)-4-isobutylpiperidine-4-carboxamide;
1-(Benzo[d][1,3]dioxol-5-ylmethyl)-N-(3,5-dimethylbenzyl)-4-isobutylpiperidine-4-carboxamide;
N-(3,5-Bis(trifluoromethyl)benzyl)-4-isobutyl-1-(2-oxo-2-sulphonylmorpholinoethyl)piperidine-4-carboxamide;
N-(3,5-Bis(trifluoromethyl)benzyl)-4-isobutyl-1-(2-morpholino-2-oxoethyl)piperidine-4-carboxamide;
N-(3,5-Bis(trifluoromethyl)benzyl)-4-((2,2-difluorocyclopropyl)methyl)-1-(4-hydroxy-3-methoxybenzyl)piperidine-4-carboxamide;
(4-Isobutyl-1-((tetrahydro-2H-pyran-4-yl)methyl)piperidin-4-yl)(4-(3-(trifluoromethyl)phenyl)piperazin-1-yl)methanone;
N-(3,5-Bis(trifluoromethyl)benzyl)-1-(4-hydroxy-4-(6-methoxypyridin-3-yl)cyclohexyl)-4-isobutylpiperidine-4-carboxamide;
N-(3,5-Bis(trifluoromethyl)benzyl)-1-(4-hydroxy-4-(6-methoxypyridin-3-yl)cyclohexyl)-4-isobutylpiperidine-4-carboxamide;
5-((4-(3,5-Bis(trifluoromethyl)benzylcarbamoyl)-4-(cyclopropylmethyl)piperidin-1-yl)methyl)-2-fluorobenzoic acid;
N-(3,5-Bis(trifluoromethyl)benzyl)-4-(cyclopropylmethyl)-1-(4-fluoro-3-(2-(pyrrolidin-1-yl)ethylcarbamoyl)benzyl)piperidine-4-carboxamide;
N-(2-(4-(3,5-Bis(trifluoromethyl)benzylcarbamoyl)-4-isobutylpiperidin-1-yl)ethyl)-6-methoxynicotinamide;
5-((4-(3,5-Bis(trifluoromethyl)benzylcarbamoyl)-4-(cyclopropylmethyl)piperidin-1-yl)methyl)-2-methoxybenzoic acid;
4-((4-(3,5-Bis(trifluoromethyl)benzylcarbamoyl)-4-(cyclopropylmethyl)piperidin-1-yl)methyl)-2-methoxybenzoic acid;
5-((4-(3,5-Bis(trifluoromethyl)benzylcarbamoyl)-4-(cyclopropylmethyl)piperidin-1-yl)methyl)-2-hydroxybenzoic acid;
N-(3,5-Bis(trifluoromethyl)benzyl)-4-(cyclopropylmethyl)-1-(3-(ethylsulfonylcarbamoyl)-4-fluorobenzyl)piperidine-4-carboxamide;
N-(3,5-Bis(trifluoromethyl)benzyl)-4-(cyclopropylmethyl)-1-(2-oxo-2-((tetrahydrofuran-2-yl)methylamino)ethyl)piperidine-4-carboxamide;
4-((4-(3,5-Bis(trifluoromethyl)benzylcarbamoyl)-4-(cyclopropylmethyl)piperidin-1-yl)methyl)-2-fluorobenzoic acid;
N-(3,5-Bis(trifluoromethyl)benzyl)-1-((6-(4-cyanophenoxy)pyridin-3-yl)methyl)-4-(cyclopropylmethyl)piperidine-4-carboxamide;
2-(2-((4-(3,5-Bis(trifluoromethyl)benzylcarbamoyl)-4-(cyclopropylmethyl)piperidin-1-yl)methyl)-6-methoxyphenoxy)acetic acid;
N-(3,5-Bis(trifluoromethyl)benzyl)-4-(cyclopropylmethyl)-1-(2-methyl-2-morpholinopropyl)piperidine-4-carboxamide;
N-(3,5-Bis(trifluoromethyl)benzyl)-4-(cyclopropylmethyl)-1-((2-morpholinothiazol-5-yl)methyl)piperidine-4-carboxamide;
1-(3-(1H-imidazol-1-yl)benzyl-N-(3,5-bis(trifluoromethyl)benzyl)-4-(cyclopropylmethyl)piperidine-4-carboxamide;
N-(3,5-Bis(trifluoromethyl)benzyl)-1-((5-(4-chlorophenyl)isoxazol-3-yl)methyl)-4-(cyclopropylmethyl)piperidine-4-carboxamide;
N-(3,5-Bis(trifluoromethyl)benzyl)-4-(cyclopropylmethyl)-1-(3-((1,3-dimethyl-1H-pyrazol-5-yl)methoxy)-4-methoxybenzyl)piperidine-4-carboxamide;
N-(3,5-Bis(trifluoromethyl)benzyl)-4-(cyclopropylmethyl)-1-((6-(thiophen-2-yl)pyridin-3-yl)methyl)piperidine-4-carboxamide;
N-(3,5-Bis(trifluoromethyl)benzyl)-4-(cyclopropylmethyl)-1-((1-(thiazol-2-yl)-1H-pyrrol-2-yl)methyl)piperidine-4-carboxamide;
N-(3,5-Bis(trifluoromethyl)benzyl)-4-(cyclopropylmethyl)-1-((3-phenylisoxazol-5-yl)piperidine-4-carboxamide;
N-(3,5-Bis (trifluoromethyl)benzyl)-4-(cyclopropylmethyl)piperidine-4-carboxamide;
N-(3,5-Bis(trifluoromethyl)benzyl)-4-(cyclopropylmethyl)-1-(3-fluoro-4-(2-(pyrrolidin-1-yl)ethylcarbamoyl)benzyl)piperidine-4-carboxamide;
N-(3,5-Bis(trifluoromethyl)benzyl)-3-(cyclopropylmethyl)pyrrolidine-3-carboxamide;
(rac) N-(3,5-Bis(trifluoromethyl)benzyl)-3-(cyclopropylmethyl)-1-(4-hydroxy-3-methoxybenzyl)pyrrolidine-3-carboxamide;
N-(3,5-Bis(trifluoromethyl)benzyl)-4-(cyclopropylmethyl)-1-(2,2-dimethyl-3-oxo-3-(thiazol-2-ylamino)propyl)piperidine-4-carboxamide;
5-((3-(3,5-Bis(trifluoromethyl)benzylcarbamoyl)-3-(cyclopropylmethyl)pyrrolidin-1-yl)methyl)-2-fluorobenzoic acid;
N-(3,5-Bis(trifluoromethyl)benzyl)-3-(cyclopropylmethyl)-1-(3-fluoro-4-methoxybenzyl)pyrrolidine-3-carboxamide;
N-(3,5-Bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-(3-fluoro-4-hydroxybenzyl)pyrrolidine-3-carboxamide;
N-(3,5-bis(trifluoromethyl)benzyl)-3-(cyclopropylmethyl)-1-(3-fluoro-4-(methylsulfonyl)benzyl)pyrrolidine-3-carboxamide;
N-(3,5-bis(trifluoromethyl)benzyl)-3-(cyclopropylmethyl)-1-((2,2-dimethylchroman-7-yl)methyl)pyrrolidine-3-carboxamide;
3-(cyclopropylmethyl)-1-((5-methoxy-2-methyl-2,3-dihydrobenzofuran-6-yl)methyl)-N-((5-(trifluoromethyl)pyridin-3-yl)methyl)pyrrolidine-3-carboxamide;
N-(3,5-bis(trifluoromethyl)benzyl)-3-(cyclopropylmethyl)-1-((5-methoxy-2-methyl-2,3-dihydrobenzofuran-6-yl)methyl)pyrrolidine-3-carboxamide or a pharmaceutically acceptable salt thereof.

21. The compound of claim 1, which is:
N-(3,5-bis(trifluoromethyl)benzyl)-3-(cyclopropylmethyl)-1-(3-(ethylsulfonylcarbamoyl)-4-fluorobenzyl)pyrrolidine-3-carboxamide;
N-(3,5-bis(trifluoromethyl)benzyl)-3-(cyclopropylmethyl)-1-(4-fluoro-3-(methylsulfonamido)benzyl)pyrrolidine-3-carboxamide;
5-((3-(3,5-bis(trifluoromethyl)benzylcarbamoyl)-3-(cyclopropylmethyl)pyrrolidin-1-yl)methyl)-2-fluorobenzoic acid;
N-(3,5-bis(trifluoromethyl)benzyl)-3-(cyclopropylmethyl)-1-(3-fluoro-4-(methylsulfonamido)benzyl)pyrrolidine-3-carboxamide;
N-(3,5-bis(trifluoromethyl)benzyl)-3-(cyclopropylmethyl)-1-(3-fluoro-4-methoxybenzyl)pyrrolidine-3-carboxamide;
N-(3,5-bis(trifluoromethyl)benzyl)-3-(cyclopropylmethyl)-1-(4-fluoro-3-(N-methylacetamido)benzyl)pyrrolidine-3-carboxamide;
N-(3,5-bis(trifluoromethyl)benzyl)-3-(cyclopropylmethyl)-1-(4-fluoro-3-methoxybenzyl)pyrrolidine-3-carboxamide;
N-(3,5-bis(trifluoromethyl)benzyl)-3-(cyclopropylmethyl)-1-((2,2-difluorobenzo[d][1,3]dioxol-5-yl)methyl)pyrrolidine-3-carboxamide;
N-(3,5-bis(trifluoromethyl)benzyl)-3-(cyclopropylmethyl)-1-(4-fluoro-3-hydroxybenzyl)pyrrolidine-3-carboxamide;
N-(3,5-bis(trifluoromethyl)benzyl)-3-(cyclopropylmethyl)-1-((3-methyloxetan-3-yl)methyl)pyrrolidine-3-carboxamide;
N-(3,5-bis(trifluoromethyl)benzyl)-3-(cyclopropylmethyl)-1-(2-methyl-2-morpholinopropyl)pyrrolidine-3-carboxamide;
N-(3,5-bis(trifluoromethyl)benzyl)-3-(cyclopropylmethyl)-1-((4′-fluorobiphenyl-4-yl)methyl)pyrrolidine-3-carboxamide;
N-(3,5-bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((3,5-dimethyltetrahydro-2H-pyran-4-yl)methyl)pyrrolidine-3-carboxamide;
N-(3,5-bis(trifluoromethyl)benzyl)-3-(cyclopropylmethyl)-1-(3-methoxytetrahydro-2H-pyran-4-yl)pyrrolidine-3-carboxamide;
N-(3,5-bis(trifluoromethyl)benzyl)-3-(cyclopropylmethyl)-1-((4,4-difluorocyclohexyl)methyl)pyrrolidine-3-carboxamide;
N-(3,5-bis(trifluoromethyl)benzyl)-3-(cyclopropylmethyl)-1-(4-hydroxy-4-(6-methoxypyridin-3-yl)cyclohexyl)pyrrolidine-3-carboxamide;
1-(4-(benzo[d][1,3]dioxol-5-yl)-4-hydroxycyclohexyl)-N-(3,5-bis(trifluoromethyl)benzyl)-3-(cyclopropylmethyl)pyrrolidine-3-carboxamide;
N-(3,5-bis(trifluoromethyl)benzyl)-4-(cyclopropylmethyl)-1-(4-hydroxy-2-methoxycyclohexyl)piperidine-4-carboxamide;
N-(3,5-bis(trifluoromethyl)benzyl)-3-(cyclopropylmethyl)-1-(4-hydroxy-2-methoxycyclohexyl)pyrrolidine-3-carboxamide;
(S)—N-(3,5-bis(trifluoromethyl)benzyl)-1-(4-hydroxy-3-methoxybenzyl)pyrrolidine-3-carboxamide;
(R)—N-(3,5-bis(trifluoromethyl)benzyl)-1-(4-hydroxy-3-methoxybenzyl)pyrrolidine-3-carboxamide;
N-(3,5-bis(trifluoromethyl)benzyl)-3-(cyclopropylmethyl)-1-((5-fluoro-1-methyl-6-oxo-1,6-dihydropyridin-3-yl)methyl)pyrrolidine-3-carboxamide;
N-(3,5-bis(trifluoromethyl)benzyl)-3-(cyclopropylmethyl)-1-((2-methoxypyridin-4-yl)methyl)pyrrolidine-3-carboxamide;
(3-(cyclopropylmethyl)-1-(4-hydroxy-3-methoxybenzyl)pyrrolidin-3-yl)(4-(4-(trifluoromethyl)pyridin-2-yl)piperazin-1-yl)methanone;
(3-(cyclopropylmethyl)-1-(4-hydroxy-3-methoxybenzyl)pyrrolidin-3-yl)(4-(3-(trifluoromethyl)phenyl)piperazin-1-yl)methanone;
N-(3,5-bis(trifluoromethyl)benzyl)-1-(4-hydroxy-3-methoxybenzyl)-3-((tetrahydro-2H-pyran-4-yl)methyl)pyrrolidine-3-carboxamide;
N-(3,5-bis(trifluoromethyl)benzyl)-1-(4-hydroxy-3-methoxybenzyl)-3-(methoxymethyl)pyrrolidine-3-carboxamide;
N-(3,5-bis(trifluoromethyl)benzyl)-1-(4-hydroxy-3-methoxybenzyl)-3-isobutylpyrrolidine-3-carboxamide;
N-(3,5-bis(trifluoromethyl)benzyl)-1-(4-hydroxy-3-methoxybenzyl)-3-(2-methoxyethyl)pyrrolidine-3-carboxamide;
5-((3-(3,5-bis(trifluoromethyl)benzylcarbamoyl)-3-(cyclopropylmethyl)pyrrolidin-1-yl)methyl)-2-fluorobenzoic acid;
N-(3,5-bis(trifluoromethyl)benzyl)-3-(cyclopropylmethyl)-1-(3-fluoro-4-methoxybenzyl)pyrrolidine-3-carboxamide;
N-(3,5-bis(trifluoromethyl)benzyl)-3-(cyclopropylmethyl)-1-(4-fluoro-3-methoxybenzyl)pyrrolidine-3-carboxamide;
N-(3,5-bis(trifluoromethyl)benzyl)-3-(cyclopropylmethyl)-1-((tetrahydrofuran-3-yl)methyl)pyrrolidine-3-carboxamide;
1-(1H-imidazol-1-yl)benzyl)-N-(3,5-bis(trifluoromethyl)benzyl)-3-(cyclopropylmethyl)pyrrolidine-3-carboxamide;
N-(3,5-bis(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((tetrahydro-2H-pyran-3-yl)methyl)pyrrolidine-3-carboxamide;
N-(3,5-bis(trifluoromethyl)benzyl)-4-(cyclopropylmethyl)-1-(4-hydroxy-2-methoxycyclohexyl)piperidine-4-carboxamide;
N-(3,5-bis(trifluoromethyl)benzyl)-3-(cyclopropylmethyl)-1-(4-hydroxy-2-methoxycyclohexyl)pyrrolidine-3-carboxamide;
(S)—N-(3,5-bis(trifluoromethyl)benzyl)-1-(4-hydroxy-3-methoxybenzyl)pyrrolidine-3-carboxamide;
(R)—N-(3,5-bis(trifluoromethyl)benzyl)-1-(4-hydroxy-3-methoxybenzyl)pyrrolidine-3-carboxamide;
N-(3,5-bis(trifluoromethyl)benzyl)-3-(cyclopropylmethyl)-1-(3-fluoro-4-hydroxybenzyl)pyrrolidine-3-carboxamide;
N-(3,5-bis(trifluoromethyl)benzyl)-3-(cyclopropylmethyl)-1-(3-fluoro-4-(methylsulfonyl)benzyl)pyrrolidine-3-carboxamide;
N-(3,5-bis(trifluoromethyl)benzyl)-3-(cyclopropylmethyl)-1-((4-methoxycyclohexyl)methyl)pyrrolidine-3-carboxamide;
N-(3,5-bis(trifluoromethyl)benzyl)-3-(cyclopropylmethyl)-1-((3-methoxytetrahydro-2H-pyran-4-yl)methyl)pyrrolidine-3-carboxamide;
N-(3,5-bis(trifluoromethyl)benzyl)-3-isopentyl-1-((tetrahydro-2H-pyran-4-yl)methyl)pyrrolidine-3-carboxamide;
N-(3,5-bis(trifluoromethyl)benzyl)-3-((2,2-difluorocyclopropyl)methyl)-1-((tetrahydro-2H-pyran-4-yl)methyl)pyrrolidine-3-carboxamide;
3-(cyclopropylmethyl)-1-((tetrahydro-2H-pyran-4-yl)methyl)-N-((5-(trifluoromethyl)pyridin-3-yl)methyl)pyrrolidine-3-carboxamide;
3-(cyclopropylmethyl)-1-((tetrahydro-2H-pyran-4-yl)methyl)-N-(1-(5-(trifluoromethyl)pyridin-3-yl)ethyl)pyrrolidine-3-carboxamide;
N-(3-cyano-5-(trifluoromethyl)benzyl)-3-(cyclopropylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((4-hydroxy-4-methylcyclohexyl)methyl)pyrrolidine-3-carboxamide;
3-benzyl-N-(3,5-bis(trifluoromethyl)benzyl)-1-((tetrahydro-2H-pyran-4-yl)methyl)pyrrolidine-3-carboxamide;
(3-(cyclopropylmethyl)-1-((tetrahydro-2H-pyran-4-yl)methyl)pyrrolidin-3-yl)(4-(4-(trifluoromethyl)pyridin-2-yl)piperazin-1-yl)methanone;
(3-(cyclopropylmethyl)-1-((tetrahydro-2H-pyran-4-yl)methyl)pyrrolidin-3-yl)(3-(4-(trifluoromethyl)pyridin-2-yl)azetidin-1-yl)methanone;
1-(4-acetamido-3-fluorobenzyl)-3-(cyclopropylmethyl)-N-45-(trifluoromethyl)pyridin-3-yl)methyl)pyrrolidine-3-carboxamide;
N-(3,5-bis(trifluoromethyl)benzyl)-3-(oxetan-3-ylmethyl)-1-((tetrahydro-2H-pyran-4-yl)methyl)pyrrolidine-3-carboxamide;
N-(3,5-bis(trifluoromethyl)benzyl)-3-(cyclobutylmethyl)-1-((tetrahydro-2H-pyran-4-yl)methyl)pyrrolidine-3-carboxamide;
N-(3,5-bis(trifluoromethyl)benzyl)-3-(cyclopropylmethyl)-1-((2,2-dimethyltetrahydro-2H-pyran-4-yl)methyl)pyrrolidine-3-carboxamide;
N-(3,5-bis(trifluoromethyl)benzyl)-3-(cyclopropylmethyl)-1-((3-methoxytetrahydrofuran-3-yl)methyl)pyrrolidine-3-carboxamide;
1-(3-acetamido-4-fluorobenzyl)-N-(3,5-bis(trifluoromethyl)benzyl)-3-(cyclopropylmethyl)pyrrolidine-3-carboxamide;
1-(4-acetamido-3-fluorobenzyl)-N-(3,5-bis(trifluoromethyl)benzyl)-3-(cyclopropylmethyl)pyrrolidine-3-carboxamide;
(3-(cyclopropylmethyl)-1-((tetrahydro-2H-pyran-4-yl)methyl)pyrrolidin-3-yl)(4-(3-(trifluoromethyl)phenyl)piperazin-1-yl)methanone;
N-(3,5-bis(trifluoromethyl)benzyl)-3-(cyclopropylmethyl)-1-(2-methoxy-2-(methoxymethyl)butyl)pyrrolidine-3-carboxamide;
N-(3,5-bis(trifluoromethyl)benzyl)-3-(cyclopropylmethyl)-1-((4-hydroxytetrahydro-2H-pyran-4-yl)methyl)pyrrolidine-3-carboxamide;
N-(3,5-bis(trifluoromethyl)benzyl)-3-(cyclopropylmethyl)-1-((4-methoxytetrahydro-2H-pyran-4-yl)methyl)pyrrolidine-3-carboxamide;
N-(3,5-bis(trifluoromethyl)benzyl)-4-(cyclopropylmethyl)-1-((2-oxo-2H-chromen-7-yl)methyl)piperidine-4-carboxamide;
or a pharmaceutically acceptable salt thereof.

22. A pharmaceutical composition, comprising a compound of claim 1 and a pharmaceutically acceptable carrier or excipient.

23. A method of treating or preventing organ transplant rejection, rheumatoid arthritis, chronic contact dermatitis, inflammatory bowel disease, lupus, systemic lupus erythematosus, multiple sclerosis, atherosclerosis, psoriasis, sarcoidosis, idiopathic pulmonary fibrosis, dermatomyositis, skin pemphigoid and related diseases, glomerulonephritides, vasculitides, hepatitis, allograft rejection, graft-versus host disease, athersclerosis, metabolic syndrome, diabetes, or obesity, comprising administering to a patient in need thereof a therapeutically effective amount of a compound of claim 1.

24. The method of claim 23, wherein said method is a method of treating or preventing multiple sclerosis, diabetes, or obesity.

25. The method of claim 23, wherein said compound is a compound of claim 16.

26. The method of claim 23, wherein said compound is a compound of claim 20.

27-30. (canceled)
